(19) United States
(12) Patent Application Publication
(10) Pub. No.: US 2022/0009959 A1
(43) Pub. Date: Jan. 13, 2022

(54) PEPTIDE LIGANDS FOR CAPTURE OF HOST CELL PROTEINS

(71) Applicant: North Carolina State University, Raleigh, NC (US)

(72) Inventors: Stefano MENEGATTI, Raleigh, NC (US); Rebecca Ashton LAVOIE, Raleigh, NC (US); Alice DI FAZIO, Raleigh, NC (US); Ruben CARBONELL, Raleigh, NC (US)

(21) Appl. No.: 17/296,795

(22) PCT Filed: Nov. 26, 2019
(86) PCT No.: PCT/US2019/063452

§ 371 Date: May 25, 2021

Related U.S. Application Data
(60) Provisional application No. 62/784,104, filed on Dec. 21, 2018.
(60) Provisional application No. 62/771,272, filed on Nov. 26, 2018.

(51) Int. Cl. C07K 1/22; C07K 16/06; C07K 7/06; B01D 15/38

(52) U.S. Cl. CPC C07K 1/22; B01D 15/3809; C07K 7/06; C07K 16/065

(57) ABSTRACT

Described are compositions and methods for removing one or more host cell proteins from a mixture. The composition comprises one or more peptides wherein each peptide in the composition has a greater binding affinity for the one or more host cell proteins than for one or more target biomolecules.

### CROSS-REFERENCE TO RELATED APPLICATIONS

This application claims priority to and the benefit of U.S. Provisional Patent Application No. 62/784,104, filed on Dec. 21, 2018, and U.S. Provisional Patent Application No. 62/771,272, filed on Nov. 26, 2018, the entire contents of each of which are fully incorporated herein by reference.

### SEQUENCE LISTING

The sequence listing is filed with the application in electronic format only and is incorporated by reference here. The sequence listing text filed “030871-9075-WO01_As_Filed_Sequence_Listing.txt” was created on Nov. 22, 2019, and is 10,241 bytes in size.

### TECHNICAL FIELD

The present disclosure relates to the development of peptide ligands for capture of host cell proteins. Specifically, the disclosure relates to development of peptide ligands for the capture and removal of host cell proteins when they are present in a mixture with target biomolecules.

### BACKGROUND

The removal of host cell proteins (HCPs) is a crucial issue in biomanufacturing, given their diversity in composition, structure, abundance, and occasional structural homology with the product. Though often referred to as a single impurity, HCPs comprise a variety of species with diverse abundance, size, function, and composition. The current approach to HCP clearance in the manufacturing of monoclonal antibodies (mAb) relies on product capture with Protein A followed by removal of residual HCPs in flow-through mode using ion exchange or mixed-mode chromatography. Recent studies, however, have highlighted the presence of “problematic HCP” species, which can degrade the mAb product or trigger immunogenic reactions, and co-elute with mAbs from Protein A and can escape capture through the polishing steps. These “problematic HCP” species compromise product stability and safety even at trace concentrations. Accordingly, effective means to improve clearance of HCPs are needed.

### SUMMARY

Disclosed herein are compositions, adsorbents and methods for removing one or more host cell proteins from a mixture wherein the mixture comprises one or more host cell proteins and one or more target biomolecules. The composition comprises one or more peptides each independently comprising a sequence selected from the group consisting of GSRYRY (SEQ ID NO: 1), RYYYAI (SEQ ID NO: 2), AAHIYY (SEQ ID NO: 3), IYRIGR (SEQ ID NO: 4), HSKIYK (SEQ ID NO: 5), ADRYGH (SEQ ID NO: 6), DRIYYY (SEQ ID NO: 7), DKQRII (SEQ ID NO: 8), RYYDYG (SEQ ID NO: 9), YRIDRY (SEQ ID NO: 10), HYAI (SEQ ID NO: 11), FRYY (SEQ ID NO: 12), HRRY (SEQ ID NO: 13), RYFF (SEQ ID NO: 14), DKSI (SEQ ID NO: 15), DRNI (SEQ ID NO: 16), HYFD (SEQ ID NO: 17), and YRFD (SEQ ID NO: 18). Each peptide in the composition has a greater binding affinity for the one or more host cell proteins than for the one or more target biomolecules.

### BRIEF DESCRIPTION OF THE DRAWINGS

FIG. 1 is a conceptual diagram of “polyclonal” synthetic HCP-binding resins. Highly specific HCP capture is not only possible, but standard practice for HCP quantification by HCP ELISA via polyclonal α-HCP antibodies, as depicted on the left. The presently used method involves the generation of a synthetic version of these polyclonal antibodies by identification of HCP-specific peptides to allow broad capture of HCPs, as shown on the right, without the expense and variability introduced by antibody-based ligands.

FIG. 2 is a graph showing the maximum fluorescent intensity (most intense pixel) distribution for fluorescently screened, manually sorted tetrameric combinatorial peptide library beads. For each bead imaged, the maximum fluorescent intensity for the IgG fluorophore (Alexa Fluor 488) is plotted against that of the HCP fluorophore (Alexa Fluor 594). Beads identified as HCP-binding ligand candidates are highlighted in the figure above, as determined by the following criteria: IgG maximum fluorescence<2,500, and HCP maximum fluorescence>10,000.

FIG. 3A and FIG. 3B are fluorescence images of unbiased combinatorial linear peptide library by ClonePix 2 on ChemMatrix HMBA resin after incubation with fluorescently tagged IgG and CHO-S HCP. In FIG. 3A, the library is imaged with ClonePix 2 FITC filter to visualize beads bound to IgG tagged with Alexa Fluor 488. FIG. 3B shows the same plate imaged with ClonePix 2 Rhodamine filter to visualize beads bound to CHO HCP tagged with Alexa Fluor 546.

FIG. 4 is a graph showing ClonePix 2 interior mean intensity (average bead intensity) distribution for hexameric combinatorial peptide library screened by ClonePix 2. For each bead imaged, the interior mean intensity for the IgG fluorophore (Alexa Fluor 488) is plotted against that of the HCP fluorophore (Alexa Fluor 546). Beads identified as HCP-binding ligand candidates are highlighted in the figure above, as determined by the following criteria: IgG maximum fluorescence<2,500, and HCP maximum fluorescence>500.

FIG. 5 is a chart showing the distribution of amino acid residues for lead tetrameric HCP-binding peptide candidates identified by manually sorted solid phase fluorescent screening by combinatorial position.

FIG. 6 is a chart showing the distribution of amino acid residues for lead hexameric HCP-binding peptide candidates identified by ClonePix 2 sorted solid phase fluorescent screening by combinatorial position.

FIG. 7A, FIG. 7B, FIG. 7C, FIG. 7D, FIG. 7E, and FIG. 7F are charts showing protein removal (N=3 for each condition) by hexameric hydrophobic positive and multipolar (6HP and 6MP, respectively) and tetrameric hydrophobic positive and multipolar (4HP and 4MP, respectively) lead HCP-binding peptide ligands coupled to Toyopearl Amino-650M resin in static binding mode, as compared to commercial resins Capto Adhere and Capto Q. Total protein removal was measured by Bradford assay. CHO-K1 host cell protein removed was measured by Cygnus CHO HCP ELISA, 3G assay kit. Monoclonal antibody removed was measured by Thermo Fisher EasyTiter kit. Each resin was screened in multiple buffer conditions (FIG. 7A=pH 6, 20 mM NaCl, FIG. 7B=pH 7, 20 mM NaCl, FIG. 7C=pH 8, 20 mM NaCl, FIG. 7D=pH 6, 150 mM NaCl, FIG. 7E=pH 7, 150 mM NaCl, FIG. 7F=pH 8, 150 mM NaCl), and at two load conditions: ˜5 mg HCP loaded per ml resin, and ˜10 mg HCP loaded per ml resin.

FIG. 8A and FIG. 8B is a table showing the data presented in FIGS. 7A-F.

FIG. 9A and FIG. 9B are bubble plot distributions of HCPs by abundance, theoretical molecular weight, theoretical isoelectric point, and grand average of hydropathy. FIG. 9A shows a host cell protein bubble plot distribution for null CHO-S clarified harvest material, used in this work as the HCP population fluorescently tagged for solid phase peptide library screening. FIG. 9B shows a host cell protein bubble plot distribution for CHO-K1 IgG-producing clarified harvest material, used in this work for secondary screening of the lead HCP-binding ligands by static binding evaluation.

FIG. 10 is a chart showing resin HCP targeted binding ratio (TBR) by resin and buffer condition (N=3). HCP TBR is defined as percent of HCP removed compared to the feed stream divided by the percent of mAb removed compared to the feed stream in static binding mode. In this analysis, HCP TBR>1 indicates preferential binding to HCP as compared to IgG, and HCP TBR<1 indicates preferential binding to IgG.

FIG. 11 is a bubble plot distribution of CHO HCP species in mAb production harvest used as load material by theoretical molecular weight (MW), isoelectric point (pI), Grand Average of Hydropathy (GRAVY), and calculated percent molar abundance. Each data point represents a unique protein identified in the GRAVY values were determined using the GRAVY Calculator. Data with the exception of GRAVY values were obtained from Thermo Proteome Discoverer.

FIG. 12A, FIG. 12B, FIG. 12C and FIG. 12D are charts showing the distribution of CHO HCPs measured in the CHO harvest load material by protein characteristic: FIG. 12A theoretical molecular weight, FIG. 12B theoretical isoelectric point, FIG. 12C theoretical grand average of hydropathy (GRAVY), a measure of relative hydrophobicity, and FIG. 12D calculated relative molar abundance.

FIG. 13 shows overlapping HCPs bound at 20 mM NaCl and 150 mM NaCl by peptide-based resins (4HP, 6HP, 4MP, and 6MP) and benchmark resins (Capto Q and Capto Adhere) at pH 6, pH 7, and pH 8. Bound proteins were determined as proteins that either were identified by LC/MS/MS in the feed but not in the supernatant samples with wash after static binding with each resin, or alternatively where the resulting spectral abundance factor was significantly lower by ANOVA (α=0.05) than the feed. The “overlap”, or number of unique species of proteins that were bound at more than one pH condition for the range tested (pH 6, 7, and 8) are shown in the overlapping regions of the Venn diagrams.

FIG. 14 shows overlapping HCPs bound at pH 6, 7, and 8 by peptide-based resins (4HP, 6HP, 4MP, and 6MP) and benchmark resins (Capto Q and Capto Adhere) at 20 mM, 150 mM. Bound proteins were determined as proteins that either were identified by LC/MS/MS in the feed but not in the supernatant samples with wash after static binding with each resin, or alternatively where the resulting spectral abundance factor was significantly lower by ANOVA (α=0.05) than the feed. The “overlap”, or number of unique species of proteins that were bound at both salt concentrations (20 mM and 150 mM) for the range tested (pH 6, 7, and 8) are shown in the overlapping regions of the Venn diagrams.

FIG. 15A and FIG. 15B shows overlapping bound proteins by peptide resins at pH 7, 20 mM NaCl. Bound proteins were determined as proteins that either were identified by LC/MS/MS in the feed but not in the supernatant samples with wash after static binding with each resin, or alternatively where the resulting dilution-adjusted spectral count was significantly lower by ANOVA (α=0.05) than the spectral count in the feed. FIG. 15A compares the number of unique species bound to the novel peptide resins (4HP, 6HP, 4MP, and 6MP) to the Capto Q benchmark resin, and FIG. 15B compares the peptide resins to the Capto Adhere benchmark resin.

FIG. 16A and FIG. 16B shows overlapping bound proteins by peptide resins at pH 6, 150 mM NaCl. Bound proteins were determined as proteins that either were identified by LC/MS/MS in the feed but not in the supernatant samples with wash after static binding with each resin, or alternatively where the resulting dilution-adjusted spectral count was significantly lower by ANOVA (α=0.05) than the spectral count in the feed. FIG. 16A compares the number of unique species bound to the novel peptide resins (4HP, 6HP, 4MP, and 6MP) to the Capto Q benchmark resin, and FIG. 16B compares the peptide resins to the Capto Adhere benchmark resin.

FIG. 17 is a table which shows tabulated spectral abundance factor and ANOVA of CHO problematic HCPs by Capto Q and HCP-binding peptide resins at pH 7, 20 mM sodium chloride. Mean and standard deviation of spectral abundance factor (N=3) are reported for each species. Calculated p-values for ANOVA comparisons of each peptide resin compared to Capto Q are provided.

FIG. 18 is a table which shows tabulated spectral abundance factor and ANOVA of CHO problematic HCPs by Capto Adhere and HCP-binding peptide resins at pH 7, 20 mM sodium chloride. Mean and standard deviation of spectral abundance factor (N=3) are reported for each species. Calculated p-values for ANOVA comparisons of each peptide resin compared to Capto Adhere are provided.

FIG. 19 is a table showing Tabulated spectral abundance factor and ANOVA of CHO problematic HCPs by Capto Q and HCP-binding peptide resins at pH 6, 150 mM sodium chloride. Mean and standard deviation of spectral abundance factor (N=3) are reported for each species. Calculated p-values for ANOVA comparisons of each peptide resin compared to Capto Q are provided.

FIG. 20 is a table showing Tabulated spectral abundance factor and ANOVA of CHO problematic HCPs by Capto Adhere and HCP-binding peptide resins at pH 6, 150 mM sodium chloride. Mean and standard deviation of spectral abundance factor (N=3) are reported for each species. Calculated p-values for ANOVA comparisons of each peptide resin compared to Capto Adhere are provided.

FIG. 21 shows the mean chromatogram (N=3) for 4MP, 6HP, and 6HP+4MP resin flow through binding at 280 nm absorbance as a function of residence time.

FIG. 22 shows the concentration of mAb in flow through fractions (N=3) by residence time and HCP-binding resin. The shaded red region indicates the mean mAb concentration±1 standard deviation in the titrated cell culture harvest feed.

FIG. 23 shows the cumulative yield of mAb product (N=3) from flow through binding with HCP selective resins as a function of resin and residence time.

FIG. 24 an example of SEC chromatogram for percent main peak, HMW % of main peak, and LMW % of main peak analysis.

FIG. 25 shows high molecular weight percent (HMW %) of main peak (N=3) from flow through binding with HCP-selective resins as a function of resin and residence time. The solid blue trend shows the measured HMW % in each fraction, while the green trend shows the calculated cumulative HMW % to simulate the HMW % of a pool of all fractions. The shaded region indicates the HMW % to main peak±1 standard deviation in the titrate cell culture harvest feed.

FIG. 26 shows low molecular weight percent (LMW %) of main peak (N=3) from flow through binding with HCP-selective resins as a function of resin and residence time. The solid blue trend shows the measured LMW % in each fraction, while the green trend shows the calculated cumulative LMW % to simulate the LMW % of a pool of all fractions. The shaded region indicates the LMW % to main peak±1 standard deviation in the titrate cell culture harvest feed.

FIG. 27 shows a table of Kruskal-Wallis H Test for bound protein isoelectric point as a function of buffer salt concentration. The distribution of isoelectric points for each unique bound protein were plotted by frequency of isoelectric point, but are not weighted based on abundance.

FIG. 28A and FIG. 28B show overlapping bound proteins by peptide resins at pH 6, 20 mM NaCl. Bound proteins were determined as proteins that either were identified by LC/MS/MS in the feed but not in the supernatant samples with wash after static binding with each resin, or alternatively where the resulting dilution-adjusted spectral count was significantly lower by ANOVA (α=0.05) than the spectral count in the feed. FIG. 28A compares the number of unique species bound to the novel peptide resins (4HP, 6HP, 4MP, and 6MP) to the Capto Q benchmark resin, and FIG. 28B compares the peptide resins to the Capto Adhere benchmark resin.

FIG. 29A and FIG. 29B. show overlapping bound proteins by peptide resins at pH 8, 20 mM NaCl. Bound proteins were determined as proteins that either were identified by LC/MS/MS in the feed but not in the supernatant samples with wash after static binding with each resin, or alternatively where the resulting dilution-adjusted spectral count was significantly lower by ANOVA (α=0.05) than the spectral count in the feed. FIG. 29A compares the number of unique species bound to the novel peptide resins (4HP, 6HP, 4MP, and 6MP) to the Capto Q benchmark resin, and FIG. 29B compares the peptide resins to the Capto Adhere benchmark resin.

FIG. 30A and FIG. 30B. show overlapping bound proteins by peptide resins at pH 7, 150 mM NaCl. Bound proteins were determined as proteins that either were identified by LC/MS/MS in the feed but not in the supernatant samples with wash after static binding with each resin, or alternatively where the resulting dilution-adjusted spectral count was significantly lower by ANOVA (α=0.05) than the spectral count in the feed. FIG. 30A compares the number of unique species bound to the novel peptide resins (4HP, 6HP, 4MP, and 6MP) to the Capto Q benchmark resin, and FIG. 30B compares the peptide resins to the Capto Adhere benchmark resin.

FIG. 31A and FIG. 31B shows overlapping bound proteins by peptide resins at pH 8, 150 mM NaCl. Bound proteins were determined as proteins that either were identified by LC/MS/MS in the feed but not in the supernatant samples with wash after static binding with each resin, or alternatively where the resulting dilution-adjusted spectral count was significantly lower by ANOVA (α=0.05) than the spectral count in the feed. Panel (A) compares the number of unique species bound to the novel peptide resins (4HP, 6HP, 4MP, and 6MP) to the Capto Q benchmark resin, and panel (B) compares the peptide resins to the Capto Adhere benchmark resin.

FIG. 32 shows values of cumulative % purity (N=3) vs. injected volume (CV) measured by SEC analysis of the flow-through fractions produced by injecting clarified CHO-K1 IgG1 production harvest titrated to pH 6 through 4MP-Toyopearl, 6HP-Toyopearl, and 4MP/6HP-Toyopearl resins at different values of residence time (0.5, 1, 2, and 5 min). The values of cumulative % purity were calculated using the following equation

$$P u r i t {y}_{C u m u l a t i v e , f} = \frac{{\sum }_{i = 1}^{f} {A}_{mAb , i}}{{\sum }_{i = 1}^{f} {A}_{HMW , i} + {A}_{mAb , i} + {A}_{LMW , i}} \times 100 \%$$

The shaded red region indicates the purity+1 standard deviation in the titrated cell culture harvest feed.

FIG. 33 shows an analysis of overlapping bound proteins present in the flow-through fractions generated by flowing clarified harvest on 6HP/4MP-Toyopearl resin at 1 minute residence time and collected at different values of column loading (CV). Bound HCPs were determined as proteins that either were identified by LC/MS/MS in the feed but not in the supernatant samples with wash after static binding with each resin, or where the resulting dilution-adjusted spectral count was significantly lower by ANOVA (α≤0.05) than the spectral count in the feed.

FIG. 34 shows an analysis of overlapping bound proteins present in the flow-through fractions generated by flowing clarified harvest on 6HP/4MP-Toyopearl resin at 2 minute residence time and collected at different values of column loading (CV). Bound HCPs were determined as proteins that either were identified by LC/MS/MS in the feed but not in the supernatant samples with wash after static binding with each resin, or where the resulting dilution-adjusted spectral count was significantly lower by ANOVA (α≤0.05) than the spectral count in the feed.

### DETAILED DESCRIPTION

Disclosed herein are methods for predicting affinity of a candidate molecule for a second molecule.

### 1. DEFINITIONS

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “an” and “the” include plural references unless the context clearly dictates otherwise. The present disclosure also contemplates other embodiments “comprising,” “consisting of” and “consisting essentially of,” the embodiments or elements presented herein, whether explicitly set forth or not.

The modifier “about” used in connection with a quantity is inclusive of the stated value and has the meaning dictated by the context (for example, it includes at least the degree of error associated with the measurement of the particular quantity). The modifier “about” should also be considered as disclosing the range defined by the absolute values of the two endpoints. For example, the expression “from about 2 to about 4” also discloses the range “from 2 to 4.” The term “about” may refer to plus or minus 10% of the indicated number. For example, “about 10%” may indicate a range of 9% to 11%, and “about 1” may mean from 0.9-1.1. Other meanings of “about” may be apparent from the context, such as rounding off, so, for example “about 1” may also mean from 0.5 to 1.4.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

### 2. COMPOSITIONS AND METHODS FOR REMOVING HOST CELL PROTEINS FROM A MIXTURE

a. Compositions

Disclosed herein are compositions for use in a method of removing one or more host cell proteins from a mixture comprising the one or more host cell proteins and one or more target biomolecules. The mixture may be any suitable mixture containing the one or more host cell proteins and the one or more target biomolecules. For example, the mixture may be cell culture fluid. For example, the mixture may be recombinant cell culture fluid. In some embodiments, the cell culture fluid may be Chinese hamster ovary (CHO) cell culture fluid. Other suitable cell culture fluids may be used in accordance with the described compositions and methods.

The composition comprises one or more peptides. Each peptide in the composition may bind with a greater affinity to the one or more host cell proteins than to the one or more target biomolecules.

The one or more target biomolecules may be any suitable target biomolecule. For example, the target biomolecule may be a protein, an oligonucleotide, a polynucleotide, a virus or a viral capsid, a cell or a cell organelle, or a small molecule. The protein may be an antibody, an antibody fragment, an antibody-drug conjugate, a drug-antibody fragment conjugate, a Fc-fusion protein, a hormone, an anticoagulant, a blood coagulation factor, a growth factor, a morphogenic protein, a therapeutic enzyme, an engineered protein scaffold, an interferon, an interleukin, or a cytokine

The one or more host cell proteins can be any host cell protein which one would want to remove from a mixture and is independently selected from the proteome of the host cell expressing the one or more target biomolecules. Examples of host cell proteins include, but are not limited to, acidic ribosomal proteins, biglycan, cathepsins, clusterin, heat shock proteins, nidogen, peptidyl-prolyl cis-trans isomerase, protein disulfide isomerase, SPARC, thrombospondin-1, vimentin, histones, endoplasmic reticulum chaperone BiP, legumain, serine protease HTRA1, and putative phospholipase B-like protein.

The one or more peptides each independently comprise a sequence selected from the group consisting of GSRYRY (SEQ ID NO: 1), RYYYAI (SEQ ID NO: 2), AAHIYY (SEQ ID NO: 3), IYRIGR (SEQ ID NO: 4), HSKIYK (SEQ ID NO: 5), ADRYGH (SEQ ID NO: 6), DRIYYY (SEQ ID NO: 7), DKQRII (SEQ ID NO: 8), RYYDYG (SEQ ID NO: 9), YRIDRY (SEQ ID NO: 10), HYAI (SEQ ID NO: 11), FRYY (SEQ ID NO: 12), HRRY (SEQ ID NO: 13), RYFF (SEQ ID NO: 14), DKSI (SEQ ID NO: 15), DRNI (SEQ ID NO: 16), HYFD (SEQ ID NO: 17), and YRFD (SEQ ID NO: 18).

One or more of the peptides may further comprise a linker on the C-terminus of the peptide. The C-terminus linker comprise a linker according to the following structure: Glyn or a [Gly-Ser-Gly]m, wherein 6≥n≥1 and 3≥m≥1. The C-terminus linker can be any suitable linker including, but not limited to GSG and GGG.

In some embodiments, each of the one or more peptides comprises a hexameric, hydrophobic/positively charged peptide (6HP) which comprises ˜25%-35% positively-charged residues (R, K, H) and 65-75% hydrophobic (I, A, F, Y) residues. Examples of these peptides include peptides independently comprising a sequence selected from the group consisting of

(SEQ ID NO: 1)
GSRYRY,
(SEQ ID NO: 2)
RYYYAI,
(SEQ ID NO: 3)
AAHIYY,
(SEQ ID NO: 4)
IYRIGR,
(SEQ ID NO: 5)
HSKIYK,
(SEQ ID NO: 19)
GSRYRYGSG,
(SEQ ID NO: 20)
RYYYAIGSG,
(SEQ ID NO: 21)
AAHIYYGSG,
(SEQ ID NO: 22)
IYRIGRGSG,
and
(SEQ ID NO: 23)
HSKIYKGSG

In another embodiment, each of the one or more peptides comprises a hexameric, multipolar peptide (6MP), which comprises one positive (R, K, H) and one negative residue (D); and (iii) hydrogen-bonding and hydrophobic peptides, which feature hydrogen bonding (Q, S, Y) and hydrophobic (I, A, F, Y) residues. Examples of these peptides include peptides independently comprising a sequence selected from the group consisting of ADRYGH (SEQ ID NO: 6), DRIYYY (SEQ ID NO: 7), DKQRII (SEQ ID NO: 8), RYYDYG (SEQ ID NO: 9), YRIDRY (SEQ ID NO: 10), ADRYGHGSG (SEQ ID NO: 24), DRIYYYGSG (SEQ ID NO: 25), DKQRIIGSG (SEQ ID NO: 26), RYYDYGGSG (SEQ ID NO: 27), and YRIDRYGSG (SEQ ID NO: 28).

In another embodiment, each of the one or more peptides comprises a tetrameric, hydrophobic/positively charged peptide (4HP) which comprises ˜25%-35% positively-charged residues (R, K, H) and 65-75% hydrophobic (I, A, F, Y) residues. Examples of these peptides include peptides independently comprising a sequence selected from the group consisting of

(SEQ ID NO: 11)
HYAI,
(SEQ ID NO: 12)
FRYY,
(SEQ ID NO: 13)
HRRY,
(SEQ ID NO: 14)
RYFF,
(SEQ ID NO: 29)
HYAIGSG,
(SEQ ID NO: 30)
FRYYGSG,
(SEQ ID NO: 31)
HRRYGSG,
and
(SEQ ID NO: 32)
RYFFGSG.

In another embodiment, each of the one or more peptides comprises a tetrameric, multipolar peptide (4MP), which comprise one positive (R, K, H) and one negative residue (D); and (iii) hydrogen-bonding and hydrophobic peptides, which feature hydrogen bonding (Q, S, Y) and hydrophobic (I, A, F, Y) residues. Examples of these peptides include peptides independently comprising a sequence selected from the group consisting of DKSI (SEQ ID NO: 15), DRNI (SEQ ID NO: 16), HYFD (SEQ ID NO: 17), YRFD (SEQ ID NO: 18), DKSIGSG (SEQ ID NO: 33), DRNIGSG (SEQ ID NO: 34), HYFDGSG (SEQ ID NO: 35), and YRFDGSG (SEQ ID NO: 36).

Some embodiments include compositions comprising one or more peptides from each of the different groups of tetrameric and hexameric and hydrophobic or multipolar peptides (4HP), (4MP), (6HP), (6MP). These peptides may be combined in the composition in any number or in any of the possible combinations from each of the groups. In one, non-limiting, embodiment, the composition comprises peptides from the 6HP and 4MP groups wherein each peptide independently comprises a sequence selected from the group consisting of GSRYRY (SEQ ID NO: 11), RYYYAI (SEQ ID NO: 2), AAHIYY (SEQ ID NO: 3), IYRIGR (SEQ ID NO: 4), HSKIYK (SEQ ID NO: 5), DKSI (SEQ ID NO: 15), DRNI (SEQ ID NO: 16), HYFD (SEQ ID NO: 17), YRFD (SEQ ID NO: 18), GSRYRYGSG (SEQ ID NO: 19), RYYYAIGSG (SEQ ID NO: 20), AAHIYYGSG (SEQ ID NO: 21), IYRIGRGSG (SEQ ID NO: 22), HSKIYKGSG (SEQ ID NO: 23), DKSIGSG (SEQ ID NO: 33), DRNIGSG (SEQ ID NO: 34), HYFDGSG (SEQ ID NO: 35), and YRFDGSG (SEQ ID NO: 36).

b. Adsorbents

Further described herein are adsorbents comprising a composition as described above, where each peptides of the composition is conjugated to a support. Supports may comprise, but are not limited to, particles, beads, plastic surfaces, resins, fibers, and/or membranes. In some embodiments, supports may include microparticles and/or nanoparticles. Each support may be made out of any suitable material including, but not limited to, synthetic or natural polymers, metals, and metal oxides. Some supports may be magnetic, such as a magnetic bead, microparticle and/or nanoparticle. Suitable synthetic polymers include, but are not limited to, polymethacrylate, polyethersulfone, and polyethyleneglycole. Suitable natural polymers include, but are not limited to, cellulose, agarose, and chitosan. Suitable metal oxides include, but are not limited to, iron oxide, silica, titania, and zirconia. Further described herein are adsorbents comprising a composition as described above conjugated to a support.

In some embodiments, the adsorbent comprises a single type of support made from a single type of support material, where all of the peptides in the composition are conjugated to supports formed of the single type of support material. In these embodiments, the composition may comprise one or more different types of peptides, each conjugated to the single type of support made from the single type of support material.

In other embodiments, the adsorbent comprises a plurality of types of support. Each type of support may be made of the same type of support material or different types of support materials. In these embodiments, the composition may comprise one or more different types of peptides, each conjugated to a different type of support.

c. Methods

The methods of the invention demonstrate improved removal of host cell proteins from a mixture compared to other methods used in the art.

Further described herein are methods for removing one or more host cell proteins from a mixture comprising the one or more host cell proteins and one or more target biomolecules. The methods comprise contacting the mixture with a composition or adsorbent described herein. In one embodiment, the contacting between the composition or adsorbent and the mixture results in the binding of the one or more host cell proteins to the composition or adsorbent. In this embodiment, the one or more host cell proteins has a higher binding affinity for the composition, as compared to the one or more target biomolecules. This results in the preferred binding of the composition to the one or more host cell proteins as compared to the one or more target molecules.

The methods of the inventions can further comprise washing the composition or adsorbent to remove one or more unbound target biomolecules into a supernatant or mobile phase; and then collecting the supernatant or mobile phase containing the one or more unbound target biomolecules. In an embodiment, the washing step can also occur after the contacting step and after the collection of the supernatant or mobile phase.

According to the methods of the invention, the method can be performed under any binding conditions suitable for use with the composition or adsorbent, including both static binding conditions and dynamic binding conditions. In some embodiments the unbound target biomolecules are collected into a supernatant when the methods are performed under static binding conditions. In some embodiments the unbound target biomolecules are collected into a mobile phase when the methods are performed under dynamic binding conditions. The methods of the invention can optionally include flow-through chromatography and weak partition chromatography.

The preferred binding affinity of the compositions and/or adsorbent for the host cell proteins, as compared to the one or more target molecules, can be altered by changes in the following: properties and concentration of the one or more target proteins; the properties and concentration of the host cell proteins; the composition, concentration, and pH of the mixture; and/or the loading conditions and residence time of the contacting and washing steps. Any of these variables can be changed to variables which are suitable according to the methods of the invention and result in increased or decreased binding affinity as required for the invention.

According to the methods of the invention, the contacting step can comprises a high ionic strength binding buffer or low ionic strength binding buffer. A low ionic strength binding buffer comprises a buffer of between 1-50 mM NaCl. In one embodiment the low ionic strength binding buffer comprises 20 mM NaCl. A high ionic strength binding buffer comprises a buffer of between 100-500 mM NaCl. In one embodiment the low ionic strength binding buffer comprises 150 mM NaCl.

According to the methods of the invention, the contacting step can comprise a low pH buffer of between pH 5-6.7.

According to the methods of the invention, the contacting step can comprise a neutral pH buffer of between pH 6.8-7.4.

According to the methods of the invention, the contacting step can comprise a high pH buffer of between pH 7.5-9.

In certain embodiments of the invention the contacting step comprise a neutral pH and low ionic strength binding buffer, wherein the buffer comprises 20 mM NaCl and has a pH of pH 7. or wherein the contacting step comprise a low pH and high ionic strength binding buffer, wherein the buffer comprises 150 mM NaCl and has a pH of pH 6. In this embodiment, each peptide can independently comprise a sequence selected from the group consisting of

(SEQ ID NO: 19)
GSRYRYGSG,
(SEQ ID NO: 20)
RYYYAIGSG,
(SEQ ID NO: 21)
AAHIYYGSG,
(SEQ ID NO: 22)
IYRIGRGSG,
(SEQ ID NO: 23)
HSKIYKGSG,
(SEQ ID NO: 33)
DKSIGSG,
(SEQ ID NO: 34)
DRNIGSG,
(SEQ ID NO: 35)
HYFDGSG,
and
(SEQ ID NO: 36)
YRFDGSG.

### 3. EXAMPLES

The accompanying Examples are offered as illustrative as a partial scope and particular embodiments of the disclosure and are not meant to be limiting of the scope of the disclosure.

### Example 1

### Design, Construction, and Screening of Solid-Phase, Combinatorial Libraries of Linear Peptides

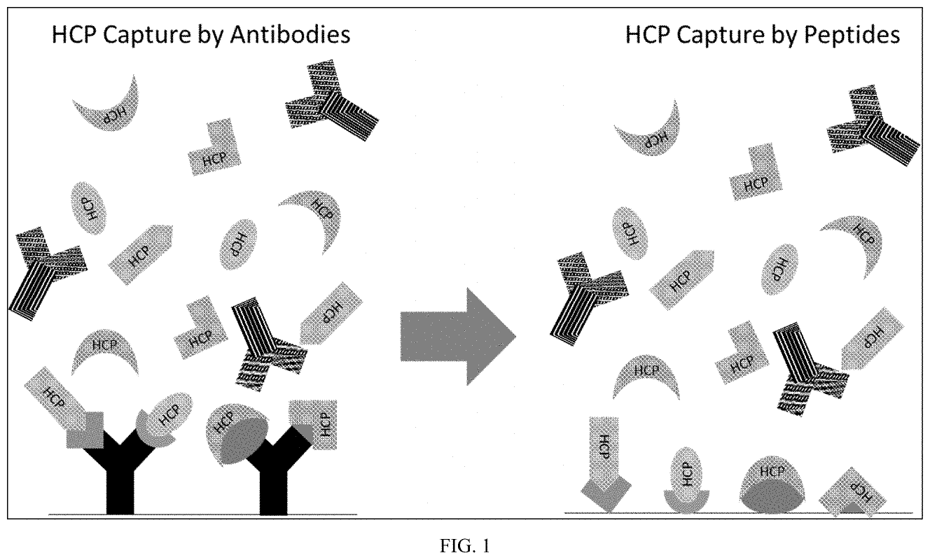

Targeted capture of hard-to-remove HR-HCPs is a promising strategy to improve product safety and efficacy. To achieve this goal, the disclosure describes the development of an ensemble of ligands capable of specific capture of HCPs in flow-through mode to be utilized as next-generation polishing media in mAb manufacturing (FIG. 1). Single ligands may either limit overall capture due to lack of promiscuous binding, or alternatively provide such broad specificity that the product also binds. As a result, the present disclosure describes the identification of multiple ligands with varied specificity towards different HCP species to balance between yield and breadth of HCP capture.

Materials: For synthesis and deprotection, the ChemMatrix HMBA resin used for library synthesis was obtained from PCAS BioMatrix (Saint-Jean-sur-Richelieu, Canada). Toyopearl AF-Amino-650M resin for secondary screening synthesis, triisopropylsilane (TIPS), and 1,2-ethanedithiol (EDT) were obtained from MilliporeSigma (St. Louis, Mo., USA). N′,N′-dimethylformamide (DMF), dichloromethane (DCM), methanol, and N-methyl-2-pyrrolidone (NMP) were obtained from Fisher Chemical (Hampton, N.H., USA). Fluorenylmethoxycarbonyl-(Fmoc-) protected amino acids Fmoc-Gly-OH, Fmoc-Ser(But)-OH, Fmoc-Ile-OH, Fmoc-Ala-OH, Fmoc-Phe-OH, Fmoc-Tyr(But)-OH, Fmoc-Asp(OtBu)-OH, Fmoc-His(Trt)-OH, Fmoc-Arg(Pbf)-OH, Fmoc-Lys(Boc)-OH, Fmoc-Asn(Trt)-OH, and Fmoc-Glu(OtBu)-OH in addition to 7-Azabenzotriazol-1-yloxy)tripyrrolidino-phosphonium hexafluorophosphate (HATU), diisopropylethylamine (DIPEA), piperidine, and trifluoroacetic acid (TFA) were obtained from Chem-Impex International (Wood Dale, Ill., USA). For peptide sequencing, citric acid, acetonitrile, and formic acid were obtained from Fisher Chemical (St. Louis, Mo., USA), ReproSil-Pur 120 C18-AQ, 3 μm resin was obtained from Dr. Maisch GmbH (Ammerbuch-Entringen, Germany), and 25 cm×100 μm PicoTip or IntegraFrit emmiter column was obtained from New Objective (Woburn, Mass., USA).

The CHO-S cell line, CD CHO AGT™ medium, CD CHO Feed A, glutamine, Pluronic F68, and Anti-Clumping Agent used to generate HCP-containing harvest for fluorescence tagging were manufactured by Life Technologies (Carlsbad, Calif., USA). Antifoam C, sodium phosphate (monobasic), and Tween 20 were obtained from MilliporeSigma (St. Louis, Mo., USA). Alexa Fluor 488, 594, and 546 NHS-Activated Esters was obtained from ThermoFisher, and sodium chloride, sodium phosphate (dibasic), sodium hydroxide, and hydrochloric acid, bis-tris, and tris were obtained from Fisher Chemical (Hampton, N.H., USA). Macrosep Advance 3 kDa MWCO Centrifugal Devices were supplied by Pall Corporation (Ann Arbor, Mich., USA), and Amicon Ultra-0.5 ml 3 kDa MWCO filters were made by EMD Millipore (St. Louis, Mo., USA). Lyophilized polyclonal human IgG was obtained from Athens Research (Athens, Ga., USA). CloneMatrix for ClonePix 2 screening was generously provided by Molecular Devices (Sunnyvale, Calif., USA). The model mAb production CHO-K1 cell culture harvest used for secondary screening was donated by a local biomanufacturing company. Capto Q and Capto Adhere chromatography resins were generously provided by GE Life Sciences (Marlborough, Mass., USA). For protein quantification, Pierce Coomassie Plus (Bradford) Assay Kits and Easy-Titer human IgG (H+L) Assay kits were obtained from Thermo Fisher (Rockford, Ill., USA). CHO HCP ELISA, 3G kits were obtained from Cygnus Technologies (Southport, N.C., USA).

Solid-Phase Peptide Synthesis and Deprotection: Solid-phase peptide synthesis (SPPS) was used for generation of both the U-CLiP libraries and identified ligands screened for this work. One-bead-one-peptide (OBOP) libraries for on-bead fluorescence screening were synthesized on ChemMatrix HMBA resin (loading=0.6 mmol amine/g resin) for the U-CLiP libraries, and lead ligand candidates for chromatographic screening were synthesized on Toyopearl Amino-650M resin (loading=0.6 mmol amine/g resin). Synthesis for all resins performed on a Syro II automated parallel peptide synthesizer (Biotage). 100 mg aliquots of resins were swelled for 20 min in DMF at 40° C. with intermediate vortexing. Couplings were performed at a 3- to 5-fold molar excess of Fmoc-protected amino acids and HATU and a 6-fold molar excess of DIPEA solubilized in NMP relative to reactive sites on the resin. The coupling reaction was performed at 45° C. for 20 minutes with agitation by intermediate vortexing. Each coupling reaction was performed 3 to 4 times per cycle prior to Fmoc deprotection to maximize reaction completion. For deprotection, resins were first washed four times with DMF, then incubated in 20% piperidine for 20 minutes at room temperature with agitation by intermediate vortexing, followed by an additional wash step as described above. All sequences were synthesized with a C-terminal glycine-serine-glycine (GSG) tail to act as a non-reactive spacer between the peptide sequence and the base matrix. Combinatorial tetrameric (X₁-X₂-X₃-X₄-G-S-G) and hexameric (X₁-X₂-X₃-X₄-X₅-X₆-G-S-G) U-CLiP libraries were synthesized as one-bead-one-peptide (OBOP) libraries using the split-couple-recombine method²⁶. For the tetrameric library, combinatorial positions were composed of equal ratios of isoleucine (I), alanine (A), glycine (G), phenylalanine (F), tyrosine (Y), aspartate (D), histidine (H), arginine (R), lysine (K), serine (S), and asparagine (N). The residues selected for the hexameric library were slightly modified by removal of F and N, and inclusion of glutamine (Q) for ease of synthesis and sequencing. Side-chain deprotection for both combinatorial libraries and single-ligand resins was performed by washing resins five times with ˜10 mL DMF, then washing the resins with ˜10 mL DCM then drying the resin with compressed nitrogen until the resin dried to a fine powder (3-5 times). A cocktail of 94% TFA, 1% EDT, 3% TIPS, and 2% deionized water was then incubated with the resin (6 ml deprotection cocktail per 100 mg resin) on a rotator at room temperature for 2 hours. Resins were washed three to five times first with DMF then 20% methanol and stored in 20% methanol at 2-8° C.

CHO-S Culture and Harvest for Host Cell Protein Production: Chinese hamster ovary (CHO) cell lines were selected as the model system to obtain typical HCP profiles found biotherapeutics processes. CHO-S cell culture harvest was donated by the Biomanufacturing Training and Education Center (BTEC) at North Carolina State University and was cultured according to their standard procedure for expansion and production of the CHO-S wild-type (WT) cell line. Briefly, the CHO cell culture bulk fluid (CCBF) was from a null CHO-S cell line grown in CD CHO AGT™ medium with 4 mM glutamine and 1 g/L pluronic F68. The cultures were fed 5% daily with CD CHO Feed A from days 3-10. The cultures are also supplemented with 0.1% Anti-Clumping Agent to prevent cell aggregation. Antifoam C was added at 10 ppm to prevent foaming in the bioreactor. CD CHO AGT™ medium contains no proteins or peptide components of animal, plant, or synthetic origin, as well as no undefined lysates or hydrolysates. The cell culture process was operated at a set pH of 7.0±0.30, 37.0° C., and 50.0% dissolved oxygen concentration. Post-production, the CHO-S harvest was clarified via centrifugation at 8,000×g for 30 min. The supernatant was then 0.2 μm filtered over a PES membrane using VWR Full Assembly Bottle-Top.

Fluorescent Labeling of IgG and CHO-S HCPs: HCPs and IgG were fluorescently label with Alexa Fluor NHS esters as guided by the manufacturer's recommendations. Briefly, wild-type CHO-S clarified harvest was concentrated to 2.3 g protein/l (˜6×) and diafiltered into 50 mM sodium phosphate, 20 mM sodium chloride, pH 8.3 using Macrosep Advance 3 kDa MWCO Centrifugal Devices. Lyophilized polyclonal human IgG (Athens Research) was dissolved in 50 mM sodium phosphate, 20 mM NaCl, pH 8.3 at a concentration of 5 g/l. 1 mg Alexa Fluor 596 NHS Ester (AF596) or Alexa Fluor 546 NHS Ester (AF546) for the HCP solution (based on the instrument to be used for fluorescence screening) and 1 mg Alexa Fluor 488 NHS Ester (AF488) for the IgG solution were each dissolved in 100 μl extra dry DMF, which was immediately combined with 1 ml of the diafiltered harvest (HCP-AF596 or HCP-AF546) or IgG (IgG-AF488) and incubated at room temperature on a rotator for 1 hour. After incubation, the samples were diafiltered into 50 mM sodium phosphate, 150 mM sodium chloride, pH 7.4 using Amicon Ultra-0.5 ml 3 kDa MWCO filters to remove unreacted Alexa Fluor dye.

Manual and High-Throughput Fluorescence Screening of Solid-Phase Peptide Libraries against IgG and CHO-S HCPs: The hexameric or tetrameric deprotected libraries were washed three times in 50 mM sodium phosphate, 150 mM sodium chloride, pH 7.4 (PBS) at 5× the settled resin volume to equilibrate. HCP-AF596 or HCP-AF546 and IgG-AF488 were diluted in 50 mM sodium phosphate, 150 mM sodium chloride, 0.2% Tween, pH 7.4 for a final concentration of ˜1.3 mg/ml IgG-AF488, ˜0.58 mg/ml HCP-AF546 or HCP-AF596, 50 mM sodium phosphate, 150 mM sodium chloride, 0.1% Tween 20 and mixed with the washed, equilibrated libraries and incubated at 2-8° C. overnight. After incubation, the excess protein solution was removed and the resin beads were washed with 50 mM sodium phosphate, 150 mM sodium chloride, 0.1% Tween 20, pH 7.4 (PBS-T). For manual fluorescence screening, the resin was aliquoted 1 bead per well in a 96-well plate in 40 μl PBS-T, then imaged by fluorescence microscopy. Lead candidate beads were selected based on highest observed intensity on the mCherry after thresholding based on GFP fluorescence.

To increase throughput, a ClonePix 2 colony picker was used for fluorescent imaging and higher throughput sorting of HCP positive and IgG negative beads in collaboration with Molecular Devices in Sunnyvale, Calif. The colony picker was identified as a possible option to increase throughput due to (1) its ability to quickly image and quantify intensity of large quantities of beads, and (2) the size range of the ChemMatrix beads, which are similar to colonies traditionally picked using the ClonePix instrument. After library incubation with fluorescently tagged proteins and washed as described above, they were suspended in a semi-solid matrix to accommodate imaging and picking. The semi-solid matrix was prepared from 2 parts Molecular Devices CloneMatrix and 3 parts 83.3 mM sodium phosphate, 250 mM NaCl, 0.17% Tween 20 to generate a matrix with buffer conditions similar to the protein binding condition used. Approximately 5 to 10 μL settled volume of incubated library was gently incorporated into the matrix solution, then evenly aliquoted across a 6-well plate to obtain a target bead density of ˜100-200 beads per well. The plates were then incubated at 37° C. for 2-18 hours to cure the matrix. Plates were imaged using the ClonePix FITC (800 ms exposure, 128 LED intensity) and Rhod (500 ms, 128 LED intensity) laser lines to monitor the presence of Alexa Fluor 488 and Alexa Fluor 546, respectively. Due to slight autofluorescence of the ChemMatrix beads under the FITC filter, bead location (i.e. ClonePix 2 run “Prime Configuration”) was assigned based on fluorescence intensity from the FITC filter. Beads were picked for further processing based on the following characteristics using the ClonePix 2: FITC interior mean intensity<2500, Rhod interior mean intensity>100, 0.05-0.25 mm radius. Picking was performed in suspension mode, with 20 μL aspiration volume to pick up the bead, and a 60 μL expel volume, where excess volume above the aspirated liquid was water.

Lead Peptide Sequencing by LC/MS/MS: Beads selected based on fluorescence were sequenced using an LC/MS/MS approach to determine lead peptide candidates for HCP-binding. Cleavage was performed as described by Kish et al²⁴. Briefly, beads that were positive for HCP fluorescence and negative for IgG fluorescence were first treated with 20 μL 0.2 M acetate, pH 3.7 for 1 hour to elute bound protein. Beads were then washed three times with deionized water, then incubated with 10 μL 38 mM sodium hydroxide, 10% v/v acetonitrile to cleave the peptide from the resin. The cleavage solution was then neutralized with 100 mM citrate buffer, 10% v/v acetonitrile, then filtered through a fritted pipette tip to remove particulate before drying the resulting solute by speed-vacuum. The powder was then resuspended in 0.1% formic acid for injection onto LC/MS/MS.

A Waters Q-ToF Premier equipped with a nanoAcquity UPLC system with a nanoflow ESI source was used for manually screened, tetrameric candidates, while a Thermo Orbitrap Elite with a Thermo EASY-nLC 1000 was used for hexameric peptide sequences from ClonePix2 screening. Chromatographic separation of the peptide samples was performed with a with a 25 cm×100 μm PicoTip or IntegraFrit emmiter column packed with ReproSil-Pur 120 C18-AQ, 3 μm resin. Samples were loaded as 10-15 μL injections and separated by a 30 min linear gradient at 300 nL/min of mobile phase A (0.1% Formic Acid) and mobile phase B (0.1% Formic Acid in acetonitrile) from 5-40% mobile phase B.

For samples sequenced by Orbitrap Elite, MS/MS sequencing was operated as follows: positive ion mode, acquisition—full scan (m/z 350-1250), 60,000 resolution, MS/MS by top 5 data dependent acquisition mode with two fragmentation events at 27 and 35 normalized collision energy (NCE) higher-energy collisional dissociation (HCD) acquisition for each interrogated precursor. Raw LC-MS data was processed using Proteome Discoverer 1.4.1.14. Searching was performed using MASCOT with a 50 ppm precursor mass tolerance and 50 ppm fragment tolerance against a FASTA formatted database of all possible peptide species in the combinatorial library. Specified modifications included dynamic modification of each amino acid residue that included a side-chain protecting group during synthesis to account for incomplete side-chain deprotection of the library.

For samples sequenced by Waters Q-ToF Premier, MS/MS sequencing was operated as follows: positive ion mode, acquisition—full scan (m/z 400-1990), MS/MS by top 8 acquisition with data dependent acquisition disabled. The default collision energy setting for the instrument based on charge state recognition was used scan collision energy based on fragmentation. Raw LC-MS data was processed using ProteinLynx Global Server 2.4. Searching was performed using MASCOT with a 50 ppm precursor mass tolerance and 50 ppm fragment tolerance against a FASTA formatted database of all possible peptide species in the combinatorial library. In cases where more than one peptide match was found for a particular bead, peptides were assigned based on the lowest expect value. Cases where this occurred generally consisted of multiple peptide identified with identical composition, but different order of amino acid residues, which is likely a result of the difficulty in distinguishing flipped combinatorial positions in a degenerate library, particularly in cases where there is low likelihood of fragmentation at particular positions.

Static Binding of HCP to Chromatographic Resins: For secondary screening, a mAb production clarified cell culture harvest derived from a CHO-K1 wild-type cell line was obtained for use as feed material. Clarified cell culture harvest was concentrated by a factor of ˜4× (˜1.2 mg/ml host cell protein) to model the expected HCP profile after initial concentration by single-pass tangential flow filtration (SPTFF) using Macrosep Advance 3 kDa MWCO Centrifugal Devices. Concentrated harvest was then diafiltered into the appropriate Bis-Tris or Tris buffer as per load condition. For pH 6 and 7 conditions, 10 mM Bis-Tris buffer solutions were used, and 10 mM Tris was used for pH 8 conditions, with “low” and “high” salt buffers composed of 20 mM NaCl and 150 mM NaCl, respectively. Lead candidate Toyopearl resins (6HP, 6MP, 4HP, 4MP) were tested alongside commercially available resins common in flow-through polishing steps for mammalian IgG production, Capto Q and Capto Adhere. Resins were aliquoted into 1 ml solid phase extraction (SPE) tubes at 25 μL settled resin volume and washed with 3×500 μL of the appropriate load buffer. Resins were then incubated with the diafiltered CHO-S harvest for 1 hour on a rotator at HCP loads of ˜5 and 10 mg HCP/mL resin and the resulting supernatant was collected. The resins were then washed with 500 μL load buffer, and the wash and flow-through samples were pooled for analysis.

Quantification of Total Protein, Host Cell Protein, and IgG Removal: Total protein concentration for samples pre- and post-treatment were measured by Bradford assay using a Pierce Coomassie Plus (Bradford) Assay Kit (Thermo Fisher, Rockford, Ill.). IgG concentration for the monoclonal IgG was determined by Thermo Scientific Easy-Titer human IgG (H+L) Assay Kit. Relative CHO HCP abundance was monitored using a Cygnus CHO HCP ELISA kit, 3G. Absolute values for HCP concentration were not determined using this assay due to the use of a general reference standard that did not account for the specific cell line or buffer condition used. To approximate HCP concentration, a correction factor was used per buffer condition to scale the observed concentrations based on the known HCP content in the feed stream. Percent removal for HCP, IgG, and total protein was calculated as follows:

$$Percent Removal = \frac{{V}_{L o a d} * {C}_{L o a d} - {V}_{F T + W a s h} * {C}_{F T + W a s h}}{{V}_{L o a d} * {C}_{L o a d}} * 100 \%$$

CHO-S Null Harvest Tabulated Host Cell Protein Identification and Relative Quantification: Species of CHO HCP are tabulated by abundance as calculated by intensity-based absolute quantification (iBAQ) as determined by proteomic identification and quantification of the null CHO-S clarified harvest material used for fluorescent screening of the solid phase combinatorial peptide library (Table 1). Concentrated, diafiltered CHO-S harvest and supernatant samples were prepared for proteomic analysis by filter-aided sample preparation (FASP) with a modified trypsin digest. For LC/MS/MS analysis, an EASY-nLC 1000 UPLC coupled to an Orbitrap Elite mass spectrometer (Thermo Scientific, San Jose, Calif.) was used. Chromatographic separation of the FASP digested samples was performed with a 25 cm×100 μm PicoTip column (New Objective, Woburn, Mass.) packed with ReproSil-Pur 120 C18-AQ, 3 μm resin (Dr. Maisch GmbH, Ammerbuch-Entringen, Germany). Samples were loaded as 15 μL injections and proteins were separated by a 120 min linear gradient at 300 nL/min of mobile phase A (0.1% formic acid in 2% acetonitrile) and mobile phase B (0.1% formic acid in acetonitrile) from 5-40% mobile phase B. The orbitrap was operated as follows: positive ion mode, acquisition—full scan (m/z 400-2000) with 60,000 resolving power, MS/MS acquisition using a top 5 data dependent acquisition implementing higher-energy collisional dissociation (HCD) with a normalized collision energy (NCE) setting of 35%. Dynamic exclusion was utilized to maximize depth of proteome coverage by minimizing re-interrogation of previously sampled precursor ions. Real-time lock mass correction using the polydimethylcyclosiloxane ion at m/z 445.120025 was utilized to minimize precursor and product ion mass measurement errors. Raw LC/MS/MS data were processed using Proteome Discoverer 1.4 (Thermo Fisher, San Jose, Calif.). Searching was performed with a 10 ppm precursor mass tolerance and 0.01 Da fragment tolerance with the Cricetus griseus subset of the UniProtKB/Swiss-Prot database with added sequence data for bovine serum albumin (acquisition ID P02769). The database search settings were specific for trypsin digestion with a maximum of 1 missed cleavage. Specified modifications included dynamic Met oxidation and static Cys carbamidomethylation. Identifications were filtered to a strict protein false discovery rate (FDR) of 1% and relaxed FDR of 5% using the Percolator node in Proteome Discoverer. Based on the sequence of each identified protein, the theoretical isoelectric point (pI) and grand average of hydropathy (GRAVY) were calculated as a model for empirical isoelectric point and hydrophobicity respectively, in addition to calculation of molecular weight (MW). GRAVY is a metric for hydrophobicity determined as the sum of the contributions of each amino acid in the protein sequence based on the water-vapor transfer free energies and interior-exterior distribution of amino acid side chains. A negative GRAVY value indicates hydrophilic character whereas a positive value indicates hydrophobicity. GRAVY values were calculated using the GRAVY Calculator developed by Stephan Fuchs at University of Greifswald. Theoretical pI and MW were calculated using the ExPASy Bioinformatics Resource Portal Compute pI/Mw tool.

TABLE 1
CHO-S Null Harvest Tabulated host Cell Protein Identification and Relative Quantification

| | | | | Percent |
|---|---|---|---|---|
| Protein | pI | GRAVY | MW (kDa) | Abundance |
| Osteopontin | 5.38 | −1.2111 | 13.1534 | 8.36% |
| Carboxypeptidase | 5.61 | −0.3493 | 51.69731 | 6.17% |
| Cathepsin B | 5.73 | −0.3481 | 35.6238 | 4.72% |
| Metalloproteinase inhibitor 1 | 8.74 | 0.0325 | 20.10075 | 3.60% |
| Cathepsin Z | 6.68 | −0.4523 | 31.48589 | 3.29% |
| Biglycan-like protein | 6.93 | −0.2041 | 39.88265 | 3.20% |
| Ribonuclease T2 | 6.25 | −0.3822 | 29.52164 | 3.06% |
| Clusterin | 5.51 | −0.6371 | 49.44111 | 2.81% |
| Peroxiredoxin-1 | 8.22 | −0.2156 | 22.24836 | 2.68% |
| C-C motif chemokine | 9.39 | −0.1601 | 13.26681 | 2.65% |
| Legumain | 5.95 | −0.3363 | 47.3632 | 2.46% |
| Cathepsin L1 | 6.72 | −0.4631 | 35.49892 | 2.20% |
| Lactadherin | 9.45 | −0.5229 | 16.14215 | 2.06% |
| Nidogen-1 | 8.41 | −0.045 | 30.07128 | 2.01% |
| Phospholipid transfer protein | 6.24 | 0.189 | 52.72359 | 1.86% |
| Sulfated glycoprotein 1 | 5.35 | 0.0285 | 27.35563 | 1.65% |
| Beta-2-microglobulin | 6.89 | −0.2613 | 13.77901 | 1.50% |
| Tissue alpha-L-fucosidase | 5.82 | −0.3539 | 50.59698 | 1.24% |
| Lactadherin (Fragment) | 6.87 | −0.2194 | 22.09951 | 1.14% |
| Glyceraldehyde-3-phosphate dehydrogenase | 8.49 | −0.0778 | 35.72522 | 1.10% |
| Galectin-1 | 5.49 | −0.2548 | 14.79318 | 1.10% |
| Galectin-3-binding protein | 5.05 | −0.0902 | 61.73522 | 1.04% |
| Peptidyl-prolyl cis-trans isomerase | 9.59 | −0.1583 | 23.61949 | 1.00% |
| Plasminogen activator inhibitor 1 | 5.83 | −0.1619 | 30.68568 | 0.94% |
| Alpha-enolase | 5.85 | −0.2166 | 46.66896 | 0.92% |
| Nidogen-1 | 4.77 | −0.3083 | 75.78242 | 0.91% |
| 14-3-3 protein epsilon | 6.04 | −0.3471 | 7.88992 | 0.90% |
| Cystatin | 9.27 | −0.2118 | 9.05565 | 0.87% |
| Cathepsin D | 6.59 | 0.0407 | 42.18856 | 0.85% |
| Nucleobindin-2-like protein | 5.1 | −1.0429 | 50.81149 | 0.83% |
| 78 kDa glucose-regulated protein | 5.01 | −0.4815 | 70.43534 | 0.74% |
| Putative out at first protein like protein (Fragment) | 5.42 | −0.1519 | 20.78159 | 0.73% |
| Nucleoside diphosphate kinase | 7.78 | −0.2928 | 17.3289 | 0.72% |
| Histone H2B | 10.2 | −0.5853 | 14.98114 | 0.70% |
| L-lactate dehydrogenase | 8.6 | −0.0128 | 42.14916 | 0.69% |
| Heat shock cognate protein | 5.37 | −0.3982 | 70.57913 | 0.68% |
| Retinoid-inducible serine carboxypeptidase | 5.3 | −0.1108 | 48.23402 | 0.67% |
| Actin, cytoplasmic 1 | 5.22 | −0.1997 | 41.71071 | 0.63% |
| Granulins | 5.97 | −0.0787 | 62.0451 | 0.63% |
| Glutathione S-transferase P | 8.24 | −0.2724 | 24.98201 | 0.62% |
| Peptidyl-prolyl cis-trans isomerase A | 8.44 | −0.3671 | 17.88783 | 0.62% |
| Nucleoside diphosphate kinase | 5.94 | −0.2533 | 17.18377 | 0.60% |
| Histone H3 | 11.1 | −0.5472 | 29.56238 | 0.56% |
| Sialidase I | 5.22 | −0.1946 | 40.61211 | 0.55% |
| Tripeptidyl-peptidase 1 | 5.77 | −0.1128 | 34.44003 | 0.55% |
| Chondroitin sulfate proteoglycan 4 | 5.38 | −0.1845 | 249.0054 | 0.53% |
| Galectin | 6.93 | −0.1427 | 32.42069 | 0.52% |
| Matrix metalloproteinase-19 | 7.81 | −0.3815 | 56.78762 | 0.50% |
| Phosphatidylethanolamine-binding protein 1 | 6.59 | −0.5963 | 20.90957 | 0.49% |
| Vimentin | 5.05 | −0.8461 | 53.71005 | 0.47% |
| Pyruvate kinase | 7.58 | −0.1261 | 51.52682 | 0.46% |
| Sulfhydryl oxidase | 8.02 | −0.2806 | 70.31 | 0.43% |
| Adipocyte enhancer-binding protein 1 | 5.11 | −0.8848 | 125.4069 | 0.43% |
| 14-3-3 protein zeta/delta | 4.73 | −0.6106 | 27.69772 | 0.41% |
| Dipeptidyl-peptidase 2 | 6.03 | −0.1374 | 52.84711 | 0.39% |
| Fibronectin | 5.44 | −0.5049 | 270.84 | 0.38% |
| Phosphoglycerate kinase 1 | 8.02 | −0.0842 | 44.53402 | 0.37% |
| Fructose-bisphosphate aldolase | 8.3 | −0.2648 | 39.35728 | 0.35% |
| Inter-alpha-trypsin inhibitor heavy chain H5 | 8.73 | −0.3249 | 101.9884 | 0.34% |
| Protein disulfide-isomerase A6 | 4.8 | −0.2753 | 28.38334 | 0.33% |
| 14-3-3 protein beta/alpha | 4.76 | −0.7402 | 27.83274 | 0.33% |
| Pigment epithelium-derived factor | 5.23 | −0.2447 | 10.5665 | 0.32% |
| Caltractin-like protein | 4.09 | −0.6537 | 16.82683 | 0.32% |
| Renin receptor | 5.03 | −0.055 | 33.8483 | 0.31% |
| Acid ceramidase | 8.13 | −0.1301 | 42.58486 | 0.31% |
| Elongation factor 1-alpha 1 | 9.1 | −0.2515 | 50.0821 | 0.31% |
| Protein disulfide-isomerase | 5.78 | −0.5002 | 54.35133 | 0.30% |
| SH3 domain-binding glutamic acid-rich-like protein | 7.8 | −0.6301 | 17.14962 | 0.30% |
| Natriuretic peptides A-like protein | 6.42 | −0.4354 | 34.13185 | 0.29% |
| Peroxiredoxin-2 | 5.12 | −0.5802 | 13.99206 | 0.29% |
| Deoxyribonuclease-2-alpha | 7.76 | −0.2946 | 38.48696 | 0.28% |
| Dickkopf-related protein 3 | 4.42 | −0.4626 | 36.05744 | 0.28% |
| 14-3-3 protein eta | 4.83 | −0.3949 | 24.41718 | 0.26% |
| Basement membrane-specific heparan sulfate | 6.41 | −0.293 | 333.9277 | 0.26% |
| proteoglycan core protein | | | | |
| Ceroid-lipofuscinosis neuronal protein 5 | 5.76 | −0.3158 | 32.04184 | 0.26% |
| Lipase | 7.43 | −0.1693 | 43.72 | 0.26% |
| Brain-specific serine protease 4-like protein | 7.02 | −0.2517 | 32.32834 | 0.25% |
| 14-3-3 protein theta | 4.68 | −0.482 | 27.73179 | 0.25% |
| Alpha-galactosidase A-like protein | 4.95 | −0.2652 | 39.73944 | 0.24% |
| Nucleobindin-2-like protein | 4.93 | −1.0615 | 52.94529 | 0.24% |
| Endoplasmin | 4.72 | −0.7189 | 90.18814 | 0.24% |
| Amyloid beta A4 protein | 5.7 | −0.5871 | 49.06087 | 0.23% |
| Beta-hexosaminidase | 6.72 | −0.2806 | 57.55518 | 0.22% |
| Acyl-CoA-binding protein | 9.33 | −0.7261 | 13.47091 | 0.22% |
| Heat shock protein HSP 90-alpha | 4.95 | −0.7347 | 84.79585 | 0.22% |
| Putative phospholipase B-like 2 | 5.63 | −0.1492 | 61.78507 | 0.21% |
| Dystroglycan | 8.7 | −0.3753 | 94.1118 | 0.21% |
| EF-HAND 2 containing protein | 5.55 | −0.3778 | 28.41752 | 0.21% |
| Glucosylceramidase | 8.11 | −0.074 | 55.2611 | 0.21% |
| Procollagen C-endopeptidase enhancer 1 | 7.94 | −0.3421 | 47.89884 | 0.21% |
| Peptidyl-prolyl cis-trans isomerase | 7.82 | −0.2123 | 18.02123 | 0.20% |
| Protein disulfide-isomerase | 4.77 | −0.4926 | 51.78094 | 0.19% |
| Insulin-like growth factor-binding protein 4 | 6.58 | −0.5425 | 25.66215 | 0.19% |
| Elongation factor 2 | 6.41 | −0.197 | 96.58058 | 0.18% |
| Transketolase | 7.56 | −0.1483 | 67.67361 | 0.17% |
| Heme oxygenase 1 | 6.14 | −0.4879 | 33.0028 | 0.17% |
| Calsyntenin-1 | 4.71 | −0.4769 | 92.22809 | 0.16% |
| Tubulointerstitial nephritis antigen-like | 6.73 | −0.5101 | 50.29258 | 0.16% |
| Procollagen-lysine,2-oxoglutarate 5-dioxygenase 1 | 5.79 | −0.4094 | 75.91806 | 0.15% |
| Phosphoglycerate mutase 1 | 7.84 | −0.5354 | 20.19257 | 0.15% |
| Beta-galactosidase (Fragment) | 6.5 | −0.1706 | 73.63046 | 0.14% |
| SPARC | 4.72 | −0.5257 | 26.25748 | 0.14% |
| Arylsulfatase A | 5 | −0.056 | 53.32317 | 0.14% |
| Myosin light polypeptide 6 | 4.56 | −0.3887 | 16.91913 | 0.14% |
| Serine protease HTRA1 | 6.54 | −0.1519 | 28.70013 | 0.14% |
| Protein-glutamine gamma-glutamyltransferase 2 | 5.11 | −0.3491 | 77.16142 | 0.14% |
| Glutathione S-transferase Mu 1-like protein | 6.23 | −0.4368 | 89.46526 | 0.14% |
| Serpin H1 | 8.62 | −0.2825 | 44.75517 | 0.13% |
| Synaptic vesicle membrane protein VAT-1-like | 7.1 | 0.0347 | 28.50465 | 0.13% |
| Heat shock protein HSP 90-beta | 5.32 | −0.5476 | 47.77726 | 0.13% |
| V-type proton ATPase subunit S1 | 5.59 | 0.1195 | 48.92993 | 0.13% |
| Aldose reductase | 7.03 | −0.2772 | 35.76849 | 0.12% |
| Hypoxanthine-guanine phosphoribosyltransferase | 6.52 | −0.0972 | 24.62769 | 0.12% |
| Lamin-A/C | 7.75 | −0.9467 | 64.00311 | 0.12% |
| Transforming growth factor beta-1-like protein (Fragment) | 5.77 | −0.293 | 16.22743 | 0.11% |
| 6-phosphogluconate dehydrogenase, decarboxylating | 6.16 | −0.176 | 53.3419 | 0.11% |
| 60S acidic ribosomal protein P2 | 4.38 | −0.2435 | 11.67384 | 0.11% |
| Fatty acid-binding protein, adipocyte | 7.7 | −0.3356 | 14.74351 | 0.11% |
| Interleukin-1 receptor-like 1 | 9.13 | −0.3903 | 35.3547 | 0.10% |
| Lipoprotein lipase | 7.94 | −0.3598 | 52.42423 | 0.10% |
| Rho GDP-dissociation inhibitor 1 | 5.1 | −0.7426 | 23.40881 | 0.10% |
| Collagen alpha-1(VI) chain | 6.11 | −0.3664 | 86.82993 | 0.10% |
| Macrophage metalloelastase | 9.38 | −0.3487 | 51.46312 | 0.09% |
| EMILIN-1 | 5.21 | −0.4179 | 104.7747 | 0.09% |
| Syndecan | 4.44 | −0.1965 | 19.99907 | 0.08% |
| Glutathione S-transferase omega-1 | 6.48 | −0.4154 | 36.53454 | 0.08% |
| Stromelysin-2 | 5.76 | −0.4462 | 105.6482 | 0.08% |
| Beta-hexosaminidase | 6 | −0.194 | 60.42712 | 0.08% |
| Moesin | 5.57 | −1.5311 | 34.50841 | 0.08% |
| Ezrin | 5.87 | −0.9875 | 68.10697 | 0.08% |
| Tropomyosin alpha-1 chain-like protein | 4.78 | −0.9069 | 36.55865 | 0.08% |
| Phosphoserine aminotransferase | 8.12 | −0.0188 | 33.90414 | 0.08% |
| N-acetylglucosamine-6-sulfatase | 6.42 | −0.4059 | 53.73742 | 0.07% |
| Protein S100-A10 | 6.27 | −0.334 | 11.23958 | 0.07% |
| Heat shock 70 kDa protein 13 | 5.78 | 0.0987 | 40.20608 | 0.07% |
| N-acetylglucosamine-1-phosphotransferase subunit gamma | 6.16 | −0.3909 | 32.46717 | 0.07% |
| Transaldolase | 6.57 | −0.268 | 37.46151 | 0.07% |
| Lysosomal alpha-glucosidase | 5.65 | −0.1717 | 105.7801 | 0.07% |
| Suprabasin | 6.83 | −0.8258 | 61.44689 | 0.07% |
| Alpha-N-acetylgalactosaminidase | 7.12 | −0.1373 | 45.2366 | 0.07% |
| Eukaryotic translation initiation factor 5A-1-like protein | 5.26 | −0.1654 | 26.04113 | 0.07% |
| Ganglioside GM2 activator | 5.72 | −0.0188 | 18.75848 | 0.06% |
| Follistatin-related protein 1 | 6.24 | −0.4027 | 65.3343 | 0.06% |
| ADP-ribosylation factor 3 | 6.84 | −0.2464 | 20.58773 | 0.06% |
| Alpha-L-iduronidase | 6.04 | −0.1523 | 67.65093 | 0.06% |
| Calcium-dependent serine proteinase | 4.72 | −0.34 | 75.13799 | 0.06% |
| Polypeptide N-acetylgalactosaminyl-transferase | 8.6 | −0.4863 | 57.30181 | 0.06% |
| Peroxidasin-like | 6.49 | −0.3424 | 162.4722 | 0.06% |
| Collagen alpha-2(VI) chain | 5.54 | −0.4214 | 84.76206 | 0.06% |
| Lysosomal Pro-X carboxypeptidase | 6.06 | −0.1321 | 53.66948 | 0.05% |
| Calumenin | 4.97 | −1.0492 | 58.15709 | 0.05% |
| Proteasome subunit alpha type | 6.45 | −0.194 | 27.86701 | 0.05% |
| Lysyl oxidase-like 1 | 6.14 | −0.5924 | 62.13942 | 0.05% |
| Protein FAM3C | 8.52 | −0.0793 | 24.73964 | 0.05% |
| Proteasome subunit alpha type-7 (Fragment) | 8.64 | −0.4137 | 23.76262 | 0.05% |
| Semaphorin-3B | 8.73 | −0.2971 | 82.976 | 0.05% |
| 40S ribosomal protein S3-like protein | 9.69 | 0.0077 | 38.84871 | 0.05% |
| Triosephosphate isomerase | 8.87 | −0.2572 | 20.39464 | 0.05% |
| Nascent polypeptide-associated complex subunit alpha, | 4.63 | −0.4188 | 34.76573 | 0.04% |
| muscle-specific form | | | | |
| Membrane frizzled-related protein isoform 1 | 5.49 | −0.1723 | 75.25366 | 0.04% |
| Adenylate kinase 2, mitochondrial | 7.7 | −0.1979 | 15.41402 | 0.04% |
| Calumenin | 4.75 | −0.7476 | 38.15948 | 0.04% |
| Peroxiredoxin-6-like protein | 6.43 | −0.1087 | 25.86053 | 0.04% |
| 60S acidic ribosomal protein P0 | 8.68 | 0.0324 | 29.86779 | 0.04% |
| MAM domain-containing protein 2 | 5.45 | −0.3531 | 53.25171 | 0.04% |
| Beta-glucuronidase | 6.15 | −0.336 | 72.48434 | 0.04% |
| Group XV phospholipase A2 | 5.86 | −0.243 | 43.52276 | 0.04% |
| Adenosylhomocysteinase | 6.09 | −0.0852 | 47.62827 | 0.04% |
| Vitamin K-dependent protein S | 5.55 | −0.2554 | 68.38178 | 0.04% |
| Matrix metalloproteinase-9 | 5.53 | −0.3708 | 76.84966 | 0.03% |
| Alpha-actinin-1 | 5.53 | −0.578 | 104.4717 | 0.03% |
| Triosephosphate isomerase | 7.62 | −0.1905 | 16.11313 | 0.03% |
| Laminin subunit beta-1 | 4.84 | −0.4547 | 199.8071 | 0.03% |
| Nucleolin | 4.43 | −1.166 | 52.4218 | 0.03% |
| Cofilin-1 | 8.22 | −0.3741 | 18.52067 | 0.03% |
| alpha-1,2-Mannosidase | 6.05 | −0.2927 | 56.0393 | 0.03% |
| Proteasome subunit alpha type | 4.74 | −0.1066 | 26.3942 | 0.03% |
| Collagen alpha-2(VI) chain | 6.08 | −0.1375 | 28.8406 | 0.03% |
| Heterogeneous nuclear ribonucleoprotein A1 | 9.17 | −0.9054 | 38.79226 | 0.03% |
| Malate dehydrogenase | 6.17 | −0.0461 | 36.44307 | 0.03% |
| Collagen alpha-1(V) chain | 5.05 | −0.94 | 45.33123 | 0.03% |
| Nucleotide exchange factor SIL1 | 4.84 | −0.1405 | 44.02121 | 0.03% |
| Polyadenylate-binding protein | 9.65 | −0.549 | 62.67493 | 0.03% |
| Tubulin beta-5 chain | 4.78 | −0.348 | 49.63897 | 0.03% |
| Protein DJ-1 | 6.32 | −0.0138 | 19.91646 | 0.03% |
| Alpha-mannosidase | 7.01 | −0.4136 | 111.2891 | 0.03% |
| Alpha-N-acetylglucosaminidase | 6.28 | −0.0411 | 81.31545 | 0.03% |
| Filamin-A | 5.64 | −0.3185 | 277.3871 | 0.03% |
| Proteasome subunit alpha type | 7.58 | −0.4674 | 29.49715 | 0.03% |
| Aldose reductase-related protein 2 | 6.23 | −0.3848 | 36.31679 | 0.03% |
| Ras-related protein Rab-7a | 6.39 | −0.3657 | 23.50287 | 0.03% |
| Latent-transforming growth factor beta-binding protein 1 | 4.99 | −0.4804 | 129.9621 | 0.03% |
| Ubiquitin-conjugating enzyme E2 N (Fragment) | 5.71 | −0.3503 | 18.58056 | 0.02% |
| Thioredoxin reductase 1, cytoplasmic | 8.23 | −0.1646 | 61.81827 | 0.02% |
| Proteasome subunit alpha type | 6.38 | −0.2494 | 29.38198 | 0.02% |
| Protein disulfide-isomerase | 8.27 | −0.7693 | 156.2722 | 0.02% |
| FK506-binding protein 9 | 4.84 | −0.1695 | 60.31894 | 0.02% |
| D-dopachrome decarboxylase | 6.57 | 0.0508 | 13.12287 | 0.02% |
| Protein SET | 4.06 | −1.3753 | 44.24729 | 0.02% |
| Thioredoxin domain-containing protein 5 | 5.25 | −0.4633 | 42.96897 | 0.02% |
| Sphingomyelin phosphodiesterase | 6.76 | −0.1683 | 69.87724 | 0.02% |
| Elongation factor 1-gamma | 6.38 | −0.47 | 49.22774 | 0.02% |
| Proteasome activator complex subunit 1 | 5.33 | −0.6665 | 24.49863 | 0.02% |
| Proteasome subunit alpha type | 6 | −0.43 | 29.51381 | 0.02% |
| Prefoldin subunit 2-like protein | 6.2 | −0.5844 | 16.68761 | 0.02% |
| Sialate O=-acetylesterase | 8.3 | −0.1415 | 59.05236 | 0.02% |
| Interferon-alpha/beta receptor beta chain | 5.22 | 0.0545 | 25.67297 | 0.02% |
| Hypoxia up-regulated protein 1 | 5.04 | −0.5876 | 108.1945 | 0.02% |
| Complement C1r-A subcomponent | 5.64 | −0.5485 | 78.12473 | 0.02% |
| Ras-related protein Rab-1B | 5.55 | −0.309 | 22.17322 | 0.02% |
| Ubiquitin-60S ribosomal protein L40-like isoform 2 | 9.87 | −0.7031 | 14.71896 | 0.02% |
| Endoplasmic reticulum resident protein 29 | 6.27 | −0.2769 | 25.63129 | 0.02% |
| Transitional endoplasmic reticulum ATPase | 5.71 | 0.022 | 237.493 | 0.02% |
| Golgi membrane protein 1 | 5.18 | −0.8608 | 40.64512 | 0.02% |
| Heterogeneous nuclear ribonucleoproteins A2/B1 | 8.53 | −0.9656 | 29.02946 | 0.02% |
| Alpha-mannosidase | 7.97 | −0.3141 | 107.548 | 0.02% |
| Cytochrome c, somatic | 9.54 | −0.7505 | 11.676 | 0.01% |
| Laminin subunit alpha-5 | 6.43 | −0.314 | 401.5767 | 0.01% |
| Metalloendopeptidase | 6.29 | −0.4376 | 111.8923 | 0.01% |
| G-protein coupled receptor 56 | 9.07 | 0.1464 | 74.47452 | 0.01% |
| Chymotrypsin-C-like protein (Fragment) | 9 | −0.4434 | 73.14142 | 0.01% |
| Septin-11 | 6.91 | −0.7062 | 50.3399 | 0.01% |
| Prostaglandin F2 receptor negative regulator | 6.13 | −0.343 | 93.6569 | 0.01% |
| Septin-2 | 5.79 | −0.524 | 37.05293 | 0.01% |
| Heterogeneous nuclear ribonucleoprotein A3 | 8.95 | −0.8475 | 26.93671 | 0.01% |
| Calreticulin | 4.34 | −1.1329 | 46.5659 | 0.01% |
| Actin-related protein 2/3 complex subunit 4 | 8.53 | −0.1327 | 19.6543 | 0.01% |
| Rab GDP dissociation inhibitor beta | 5.93 | −0.3146 | 50.5347 | 0.01% |
| Alpha-mannosidase | 7.2 | −0.3441 | 125.2873 | 0.01% |
| Isocitrate dehydrogenase [NADP] | 6.53 | −0.3944 | 46.67652 | 0.01% |
| Vasorin | 7.61 | −0.1282 | 69.93944 | 0.01% |
| Putative costars family protein | 8.76 | −0.4227 | 17.51608 | 0.01% |
| Neuroblast differentiation-associated protein AHNAK-like | 5.85 | −0.4226 | 130.9968 | 0.01% |
| protein (Fragment) | | | | |
| Peptidyl-glycine alpha-amidating monooxygenase B | 5.94 | −0.2955 | 92.32074 | 0.01% |
| NSFL1 cofactor p47 | 5.1 | −0.6586 | 40.98453 | 0.01% |
| Ras-related protein Rab-5C | 8.64 | −0.3093 | 23.46881 | 0.01% |
| Amine oxidase | 6.06 | −0.3141 | 77.25271 | 0.01% |
| Collagen alpha-1(XVI) chain | 8.21 | −0.7206 | 156.0084 | 0.01% |
| Farnesyl pyrophosphate synthetase | 7.26 | −0.2752 | 40.9039 | 0.01% |
| Clathrin heavy chain 1-like protein | 5.86 | −0.2297 | 265.1194 | 0.01% |
| T-complex protein 1 subunit delta | 8.55 | 0.1365 | 42.10956 | 0.01% |
| Heterogeneous nuclear ribonucleoprotein D0 | 9.34 | −1.0233 | 29.26428 | 0.01% |
| Sushi repeat-containing protein SRPX | 8.75 | −0.2106 | 37.86603 | 0.01% |
| T-complex protein 1 subunit theta | 4.77 | −0.2834 | 22.20929 | 0.01% |
| CMP-N-acetylneuraminate-beta-galactosamide-alpha-2, 3- | 9.45 | −0.2153 | 38.914 | 0.01% |
| sialyltransferase | | | | |
| F-actin-capping protein subunit alpha-1 | 6.23 | −0.6069 | 26.80543 | 0.01% |
| T-complex protein 1 subunit alpha | 5.7 | −0.0254 | 60.30065 | 0.01% |
| Multiple inositol polyphosphate phosphatase 1-like protein | 8.67 | −0.5433 | 60.49575 | 0.01% |
| Heterogeneous nuclear ribonucleoprotein C-like 1-like isoform 3 | 4.91 | −0.9363 | 34.45093 | 0.01% |
| 40S ribosomal protein SA | 9.36 | −0.0235 | 19.7215 | 0.01% |
| Ras-related protein Rab-6A-like protein | 5.23 | −0.4264 | 22.89755 | 0.01% |
| Vinculin | 5.44 | −0.3917 | 124.7994 | 0.01% |
| ATP-citrate synthase | 6.03 | −0.169 | 77.13236 | 0.01% |
| LIM and SH3 domain protein 1 | 5.68 | −1.1604 | 25.8065 | 0.01% |
| Keratin, type I cytoskeletal 10 (Fragment) | 4.43 | −0.7185 | 27.83584 | 0.01% |
| Aspartate aminotransferase | 6.73 | −0.2455 | 46.22369 | 0.01% |
| Neural cell adhesion molecule 1 | 4.72 | −0.3599 | 114.2459 | 0.01% |
| Fumarylacetoacetase | 6.53 | −0.2639 | 40.76139 | 0.01% |
| Filamin-B | 5.46 | −0.2833 | 277.6718 | 0.01% |
| Guanine nucleotide-binding protein subunit beta-2-like 1 | 7.03 | −0.2583 | 30.4372 | 0.01% |
| Palmitoyl-protein thioesterase 1 | 7.73 | −0.0905 | 32.33334 | 0.01% |
| Serpin B6 | 5.67 | −0.2466 | 43.05545 | 0.01% |
| Elongation factor 1-delta-like isoform 1 | 6.66 | −0.7434 | 72.92385 | 0.01% |
| SWI/SNF complex subunit SMARCC2 | 5.23 | −0.7755 | 121.915 | 0.01% |
| Heterogeneous nuclear ribonucleoprotein A/B | 9.62 | −0.9161 | 23.73917 | 0.00% |
| Purine nucleoside phosphorylase-like protein | 6.08 | −0.2509 | 38.00292 | 0.00% |
| Importin subunit beta-1 | 4.84 | −0.1319 | 175.8534 | 0.00% |
| Pirin-like protein | 6.01 | −0.5593 | 32.06016 | 0.00% |
| Tryptophanyl-tRNA synthetase, cytoplasmic | 5.99 | −0.3922 | 53.50173 | 0.00% |
| Cytosolic non-specific dipeptidase | 5.63 | −0.3029 | 52.80857 | 0.00% |
| A disintegrin and metalloproteinase with thrombospondin | 5.4 | −0.5748 | 149.4347 | 0.00% |
| motif 7-like protein (Fragment) | | | | |
| Prostaglandin reductase 1-like protein | 6.48 | −0.1288 | 43.32088 | 0.00% |
| Kinesin-like protein | 6.11 | −0.7322 | 185.594 | 0.00% |
| Periaxin (Fragment) | 8.26 | −0.4536 | 187.5386 | 0.00% |
| Glucose-6-phosphate isomerase | 7.08 | −0.3224 | 63.01916 | 0.00% |
| Far upstream element-binding protein 2 | 7.61 | −0.7103 | 62.15974 | 0.00% |
| Adenylosuccinate lyase | 6.42 | −0.2182 | 54.9182 | 0.00% |
| Non-specific lipid-transfer protein | 7.17 | −0.196 | 58.85462 | 0.00% |
| Apoptosis-inducing factor 1 | 8.93 | −0.2286 | 66.07947 | 0.00% |
| Plectin (Fragment) | 5.58 | −0.6836 | 508.7089 | 0.00% |
| Glyoxalase domain-containing protein 4 | 5.28 | −0.397 | 33.19359 | 0.00% |
| Glutathione synthetase | 5.42 | −0.2015 | 52.15582 | 0.00% |
| Nuclear migration protein nudC-like protein | 5.33 | −1.0538 | 38.28532 | 0.00% |
| Cullin-associated NEDD8-dissociated protein 1 | 5.49 | −0.0118 | 133.5373 | 0.00% |
| Myosin-9 | 5.77 | −0.7296 | 268.4091 | 0.00% |
| Semaphorin-3C | 7.66 | −0.2514 | 63.09164 | 0.00% |
| Dipeptidyl peptidase 3-like protein | 5.2 | −0.3706 | 85.65625 | 0.00% |
| E3 ubiquitin-protein ligase | 6.51 | −0.2262 | 53.362 | 0.00% |
| Soluble calcium-activated nucleotidase 1 | 6.16 | −0.4085 | 45.20392 | 0.00% |
| Semaphorin-4B | 6.93 | −0.2201 | 87.83328 | 0.00% |
| Chloride intracellular channel protein | 5.09 | −0.2934 | 26.93576 | 0.00% |
| Glucose-6-phosphate 1-dehydrogenase | 8.37 | 0.0101 | 145.0064 | 0.00% |
| Plastin-3 | 5.29 | −0.321 | 74.6751 | 0.00% |
| Ras GTPase-activating-like protein IQGAP1 | 6.25 | −0.478 | 187.9681 | 0.00% |
| Neogenin | 6.17 | −0.37 | 154.3812 | 0.00% |
| Cytosolic acyl coenzyme A thioester hydrolase | 7.17 | −0.4012 | 35.62203 | 0.00% |
| Prolow-density lipoprotein receptor-related protein 1 | 5.13 | −0.5026 | 494.439 | 0.00% |
| Laminin subunit gamma-1 | 4.96 | −0.6118 | 172.0111 | 0.00% |
| Tenascin | 5.35 | −0.4568 | 286.3209 | 0.00% |
| Vacuolar protein sorting-associated protein 35 | 5.28 | −0.3058 | 91.75188 | 0.00% |
| Coiled-coil domain-containing protein 80 | 9.69 | −0.7878 | 104.7633 | 0.00% |
| Alpha-1,6-mannosylglycoprotein 6-beta-N- | 8.55 | | 84.50603 | 0.00% |
| acetylglucosaminyltransferase A | | | | |
| Importin-5 | 4.85 | −0.1296 | 89.65504 | 0.00% |
| Eukaryotic translation initiation factor 3 subunit A | 6.38 | −1.3973 | 166.3715 | 0.00% |
| Protein OS-9-like isoform 1 | 4.84 | −0.9262 | 75.44049 | 0.00% |
| Stress-70 protein, mitochondrial | 5.87 | −0.4087 | 73.68478 | 0.00% |
| CAD protein | 6.06 | −0.0724 | 242.8766 | 0.00% |
| Hexokinase | 6.21 | −0.2112 | 102.243 | 0.00% |
| Thimet oligopeptidase | 5.66 | −0.4311 | 78.05127 | 0.00% |
| von Willebrand factor A domain-containing protein 5A | 5.54 | −0.208 | 79.55311 | 0.00% |
| RuvB-like 1 | 6.02 | −0.2513 | 50.1823 | 0.00% |
| Fatty acid synthase | 5.95 | −0.0823 | 271.6874 | 0.00% |
| Xanthine dehydrogenase/oxidase-like protein | 7.45 | −0.1926 | 146.348 | 0.00% |

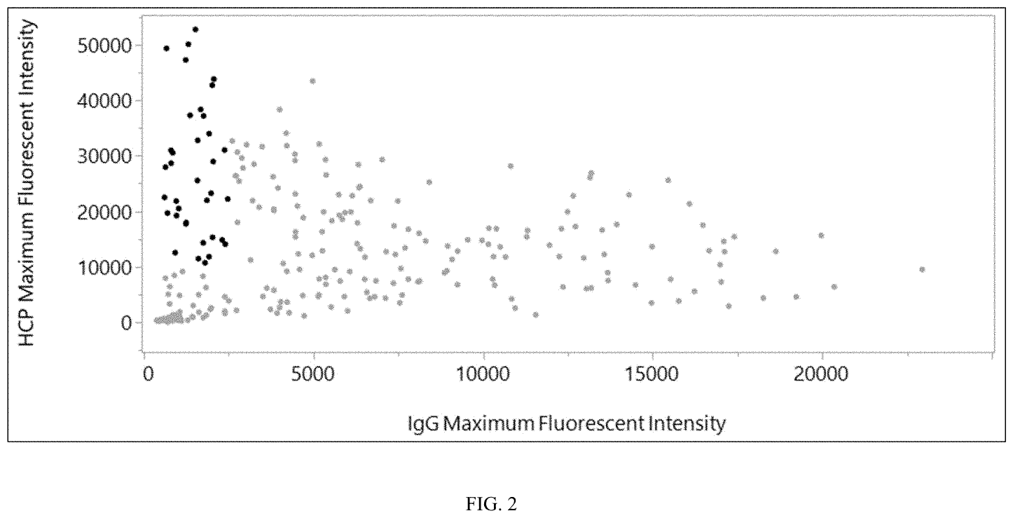

mAb harvests, to favor the selection of ligands with high HCP binding activity. A volume of ˜5 μL of settled ChemMatrix library resin beads was combined with 10 μL fluorescent protein and incubated overnight at 2-8° C. to ensure saturation of the resin beads. An aliquot of 288 library beads were sampled from the tetrameric X₁X₂X₃X₄GSG library and individually plated into 96-well plates. After imaging each bead by fluorescence microscopy, the distribution of the maximum fluorescent intensity, or most intense pixel, for emission from Alexa Fluor 488 (IgG) compared to Alexa Fluor 594 (HCP) was assessed, as shown in FIG. 2.

Beads were selected by applying the following criteria: (i) IgG maximum fluorescence<2,500, based on observed the fluorescent intensity range from negative control beads; (ii) HCP maximum fluorescence Library Design and Synthesis: The OBOP peptide libraries used for this work were synthesized using the split-couple-recombine method to discover synthetic ligands that bind target proteins. Libraries were synthesized on ChemMatrix resin, which affords high peptide purity and can be used to probe protein binding. Given that the majority of HCPs present in the CHO harvest material are hydrophilic and negatively charged at physiological conditions, the amino acid composition was limited to 12 out of the 20 natural amino acids for library construction, namely histidine, arginine, and lysine (positively charged); isoleucine, alanine, and glycine (aliphatic); phenylalanine and/or tyrosine (aromatic), aspartate (negatively charged), serine, and asparagine or glutamine (polar). Notably, narrowing the pool of amino acids reduces library size and screening time, and aids sequencing. Two libraries were constructed, namely a tetrameric X₁X₂X₃X₄GSG and a hexameric X₁X₂X₃X₄X₅X₆GSG, wherein Xᵢ represent a combinatorial position that can be occupied by any of the chosen amino acids, and GSG is a Gly-Ser-Gly C-terminal spacer. Hexamers are effective small synthetic ligands for pseudo-affinity and low concentration applications. In addition, shorter tetrapeptides were utilized to determine whether comparable capacity and specificity could be obtained at a lower cost-of-goods. The GSG spacer included in the library sequence was used as an inert spacer arm to promote the display of the combinatorial segment, and was used as a tracking sequence in LC/MS/MS peptide sequencing due to frequent occurrence of both the -GSG and -SG y-ion fragments observed. HMBA ChemMatrix resin was selected for this work, where the hydroxymethylbenzoic acid (HMBA) linker on this resin allows for on-resin deprotection of the side chain functional groups on the amino acid residues prior to library screening; the linker is also alkaline-labile, and enables post-screening cleavage of the peptides from the selected ChemMatrix beads to be finally sequenced by LC/MS/MS.

Manual tetrameric library screening and detection of CHO HCP specificity by fluorescence detection: During the initial screening of the OBOP combinatorial libraries, it was sought to demonstrate the value of simultaneous positive/negative screening with fluorescent labels for identifying HCP-selective peptide binders. Ligand identification by binding a fluorescently labelled target is beneficial for its potential for high-throughput sorting and its compatibility with simultaneous positive and negative screening. The HCP targets have a very broad range of molecular weights. Alexa Fluor fluorescent dyes were chosen owing to their high fluorescence and photo-stability. Alexa Fluor 488 was used for IgG labelling and AlexaFluor 594 or 546 was used for HCP labelling to ensure minimal overlap of emission and compatibility with instrumentation. The labelled proteins were combined in a ˜1:3 HCP:IgG ratio, which is higher than the protein makeup in typical >10,000, to include the upper 50% of beads by HCP max intensity (one-sided upper tolerance interval˜13,500, α=0.95). Radial fluorescent intensity for each wavelength was also tracked to establish typical patterning observed for the beads selected, to establish manual verification of the selected beads to ensure the maximum fluorescence signal was not a result of an image artifact or bead defect. This resulted in ˜20% of the bead population selected for sequencing.

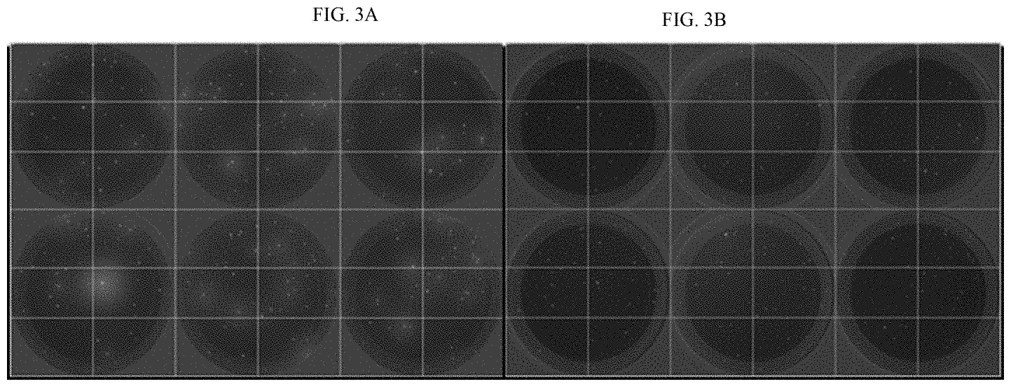

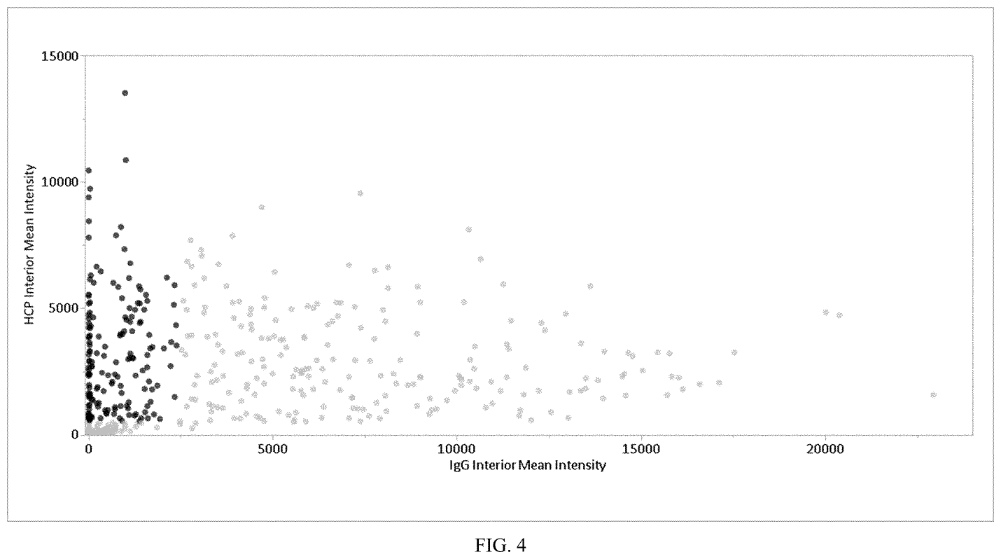

ClonePix 2 Hexameric Library Sorting and Detection of CHO HCP Specificity by Fluorescence Detection: The bead sorting criteria defined through manual sorting were implemented to automate the screening of ˜7,000 beads randomly sampled from the X₁X₂X₃X₄X₅X₆GSG library using a ClonePix 2 machine (Molecular Devices, Sunnyvale, Calif.). For the ClonePix 2 system, bead selection was based on the interior mean intensity parameter developed for the ClonePix system, which is approximately equivalent to average fluorescent intensity within the bounds of the beads shown in FIG. 3A and FIG. 3B. Beads were selected based on the following gates: (i) FITC (green) interior mean intensity<2,500; (ii) Rhodamine (red) interior mean intensity>500, representing a similar ratio of picked beads to the total beads screened (˜20%). While the threshold for bead selection for the HCP fluorescence in this instance may appear substantially lower than observed with the manual screening, differences were expected given that a different Alexa Fluor dye was required for this system (Alexa Fluor 546, which has a lower reported initial brightness compared to Alexa Fluor 594), in addition to differences in imaging exposure and intensity required to visualize the beads. The interior mean intensity characteristic of the picked beads is shown in FIG. 4.

Sequencing of HCP-Binding Ligand Candidates: The selected beads were processed for peptide sequencing. First, the isolated beads were copiously rinsed with 0.2 M acetate buffer, pH 3.7 to remove all bound proteins. Particular care was taken with the beads selected with the ClonePix 2 device to remove the CloneMatrix utilized to immobilize the beads for imaging and picking. The beads were then individually treated with 38 mM sodium hydroxide, 10% v/v acetonitrile to cleave the ester bond between the GSG spacer and the HMBA linker; to prevent alkaline degradation of the peptide, the exposure to the alkaline solution was limited to 10 min, after which the cleavage solutions was neutralized with an equal volume of 100 mM citrate buffer, 10% v/v acetonitrile. The cleaved peptides were then reconstituted in aqueous 0.1% formic acid and sequenced by liquid chromatography electrospray ionization tandem mass spectrometry (LC-ESI-MS/MS). The peptide sequences were obtained by searching the acquired MS data against the corresponding tetramer and hexamer peptide FASTA databases using MASCOT (Matrix Science).

The resulting sequences, listed in Table 2, were grouped in three classes based on consensus in amino acid composition, namely (i) hydrophobic/positively charged peptides (HP), which comprise ˜25%-35% positively-charged residues (R, K, H) and 65-75% hydrophobic (I, A, F, Y) residues; (ii) multipolar peptides (MP), which comprise one positive (R, K, H) and one negative residue (D); and (iii) hydrogen-bonding and hydrophobic peptides, which feature hydrogen bonding (Q, S, Y) and hydrophobic (I, A, F, Y) residues. Identification and quantification of CHO HCPs are shown in Table 1. The majority of the HCPs have sequence-based isoelectric points<7, and are likely negatively charged under physiological conditions. Thus, the consistent identification of peptides featuring positive amino acids is consistent with capture of these species via long-range ionic interactions.

The sequences specified here were sequenced by comparison of LC/MS/MS spectra to a FASTA sequence library of all possible peptide sequences in the combinatorial library from the combinatorial library beads that were identified as HCP-positive and IgG-negative solid phase fluorescent screening studies.

TABLE 2
Lead HCP-binding peptide candidates.

| | Positive/hydrophobic | Multipolar | Hydrogen bonding/hydrophobic |
|---|---|---|---|
| Hexameric | AAHIYY-GSG (SEQ ID NO: 3) | ADRYGH-GSG (SEQ ID NO: 6) | AAIIYY-GSG (SEQ ID NO: 3) |
| | GSRYRY-GSG (SEQ ID NO: 1) | DKQRII-GSG (SEQ ID NO: 8) | GEDQYY-GSG (SEQ ID NO: 48) |
| | HSKIYK-GSG (SEQ ID NO: 5) | DRIYYY-GSG (SEQ ID NO: 7) | HQASSQ-GSG (SEQ ID NO: 49) |
| | IYRIGR-GSG (SEQ ID NO: 4) | RYYDYG-GSG (SEQ ID NO: 9) | QQYIII-GSG (SEQ ID NO: 50) |
| | RYYYAI-GSG (SEQ ID NO: 2) | YRIDRY-GSG (SEQ ID NO: 10) | |
| Tetrameric | AFNA-GSG (SEQ ID NO: 37) | DKSI-GSG (SEQ ID NO: 15) | AIYF-GSG (SEQ ID NO: 51) |
| | KFFF-GSG (SEQ ID NO: 38) | DRNI-GSG (SEQ ID NO: 16) | NYRS-GSG (SEQ ID NO: 52) |
| | AFYH-GSG (SEQ ID NO: 39) | HYFD-GSG (SEQ ID NO: 17) | DFNY-GSG (SEQ ID NO: 53) |
| | KYGY-GSG (SEQ ID NO: 40) | YRFD-GSG (SEQ ID NO: 18) | GSIG-GSG (SEQ ID NO: 54) |
| | FRYY-GSG (SEQ ID NO: 12) | | GSSY-GSG (SEQ ID NO: 55) |
| | KYFF-GSG (SEQ ID NO: 41) | | GFYG-GSG (SEQ ID NO: 56) |
| | HFFA-GSG (SEQ ID NO: 42) | | IAFG-GSG (SEQ ID NO: 57) |
| | HFIF-GSG (SEQ ID NO: 43) | | IYYA-GSG (SEQ ID NO: 58) |
| | RYFF-GSG (SEQ ID NO: 14) | | SYIY-GSG (SEQ ID NO: 59) |
| | HNFI-GSG (SEQ ID NO: 44) | | YAFG-GSG (SEQ ID NO: 60) |
| | YRFF-GSG (SEQ ID NO: 45) | | |
| | YYFR-GSG (SEQ ID NO: 46) | | |
| | HYAI-GSG (SEQ ID NO: 11) | | |
| | HYFR-GSG (SEQ ID NO: 47) | | |
| | HRRY-GSG (SEQ ID NO: 13) | | |

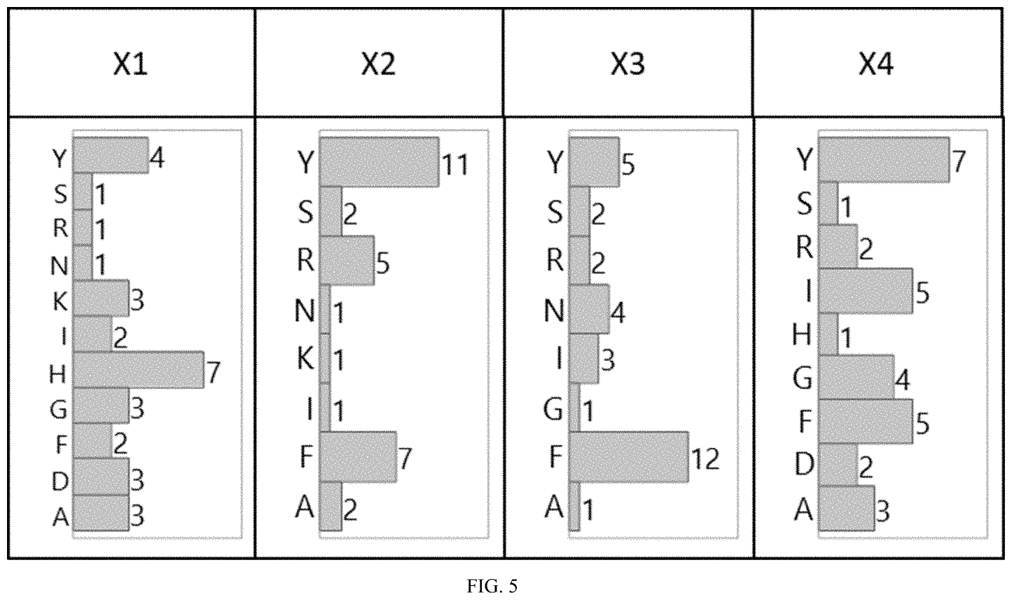

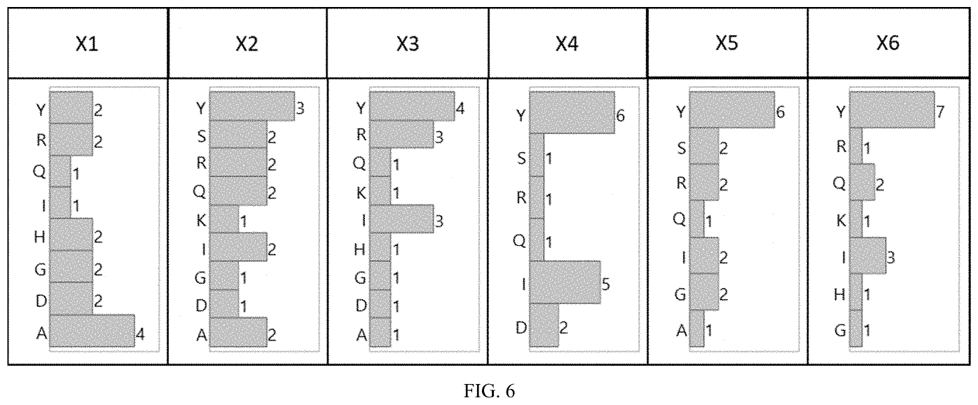

The distribution of the amino acids by combinatorial position, shown in FIG. 5 (tetrameric) and FIG. 6 (hexameric), reveal preferential placement of hydrophobic, particularly aromatic, amino acids towards the C-terminus. This phenomenon, which is especially apparent with hexameric sequences, can be attributed to a sequence-based peptide-HCP affinity across multiple HCP species, or to an unexpected bias in the libraries related to a higher synthetic yield of the observed sequences. The consensus observed within each library and between the two libraries, however, indicates limited bias in either bead selection or sequencing introduced between the two screening methods (manual sorting vs. ClonePix 2 sorting) used for this work.

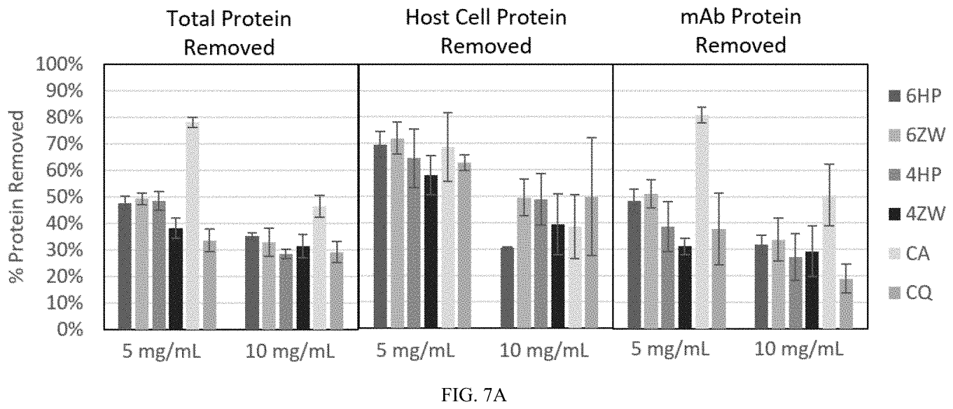

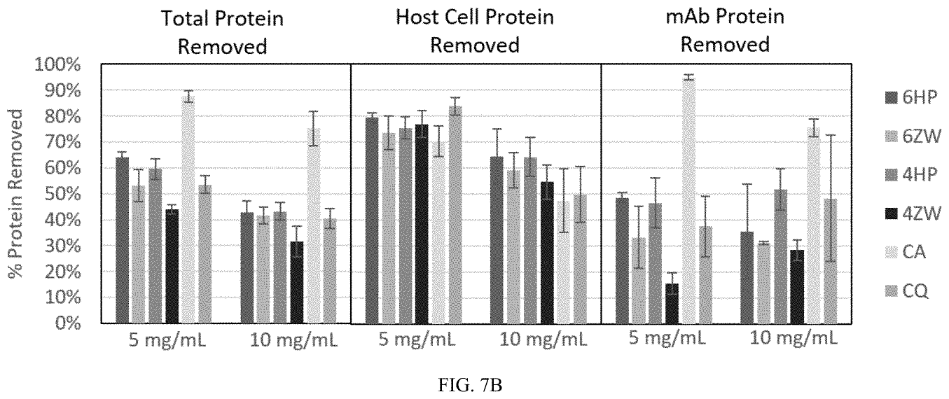

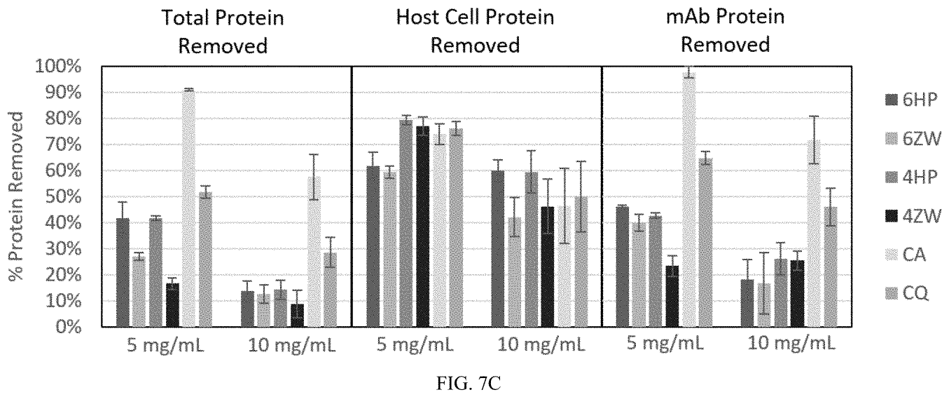

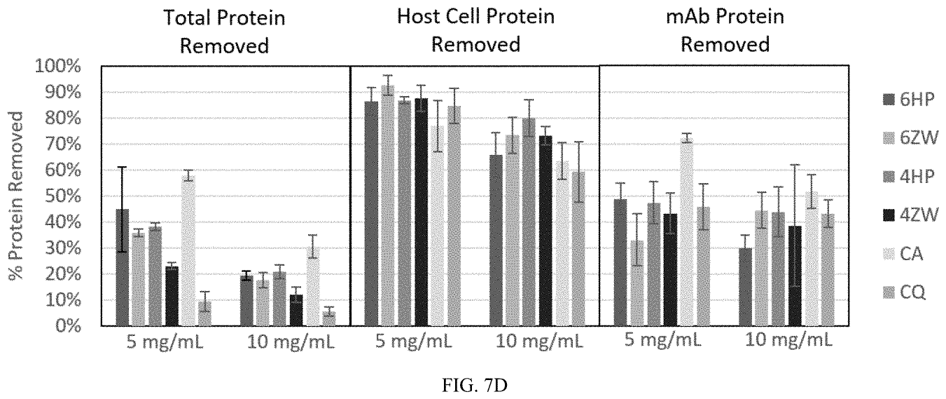

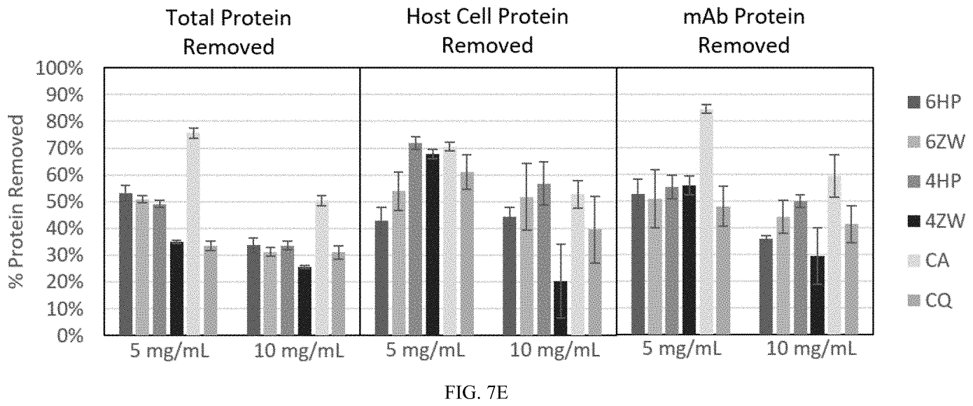

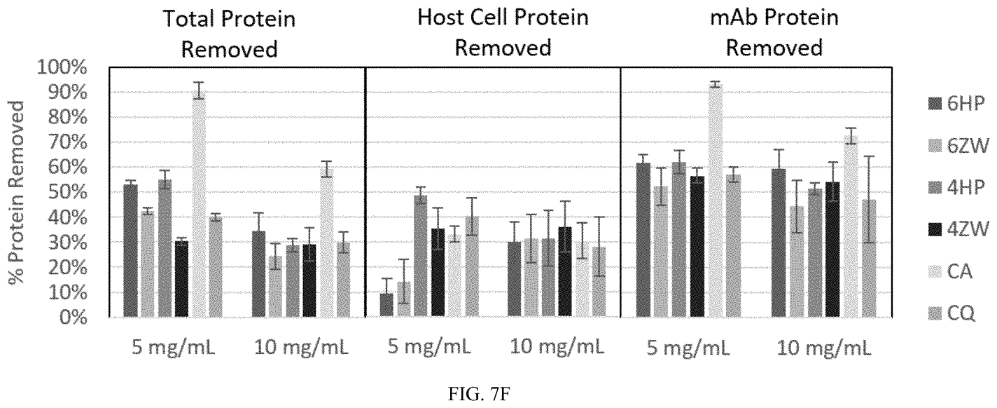

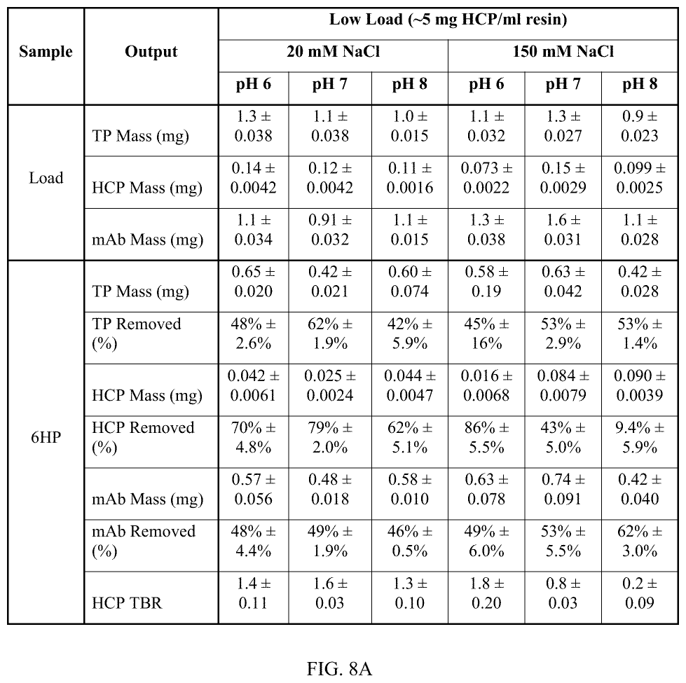

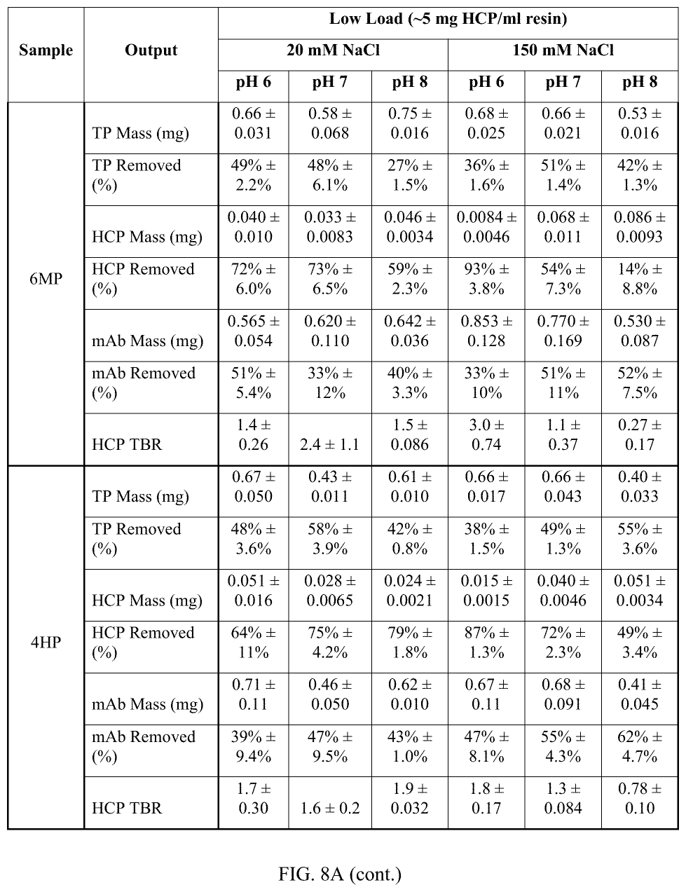

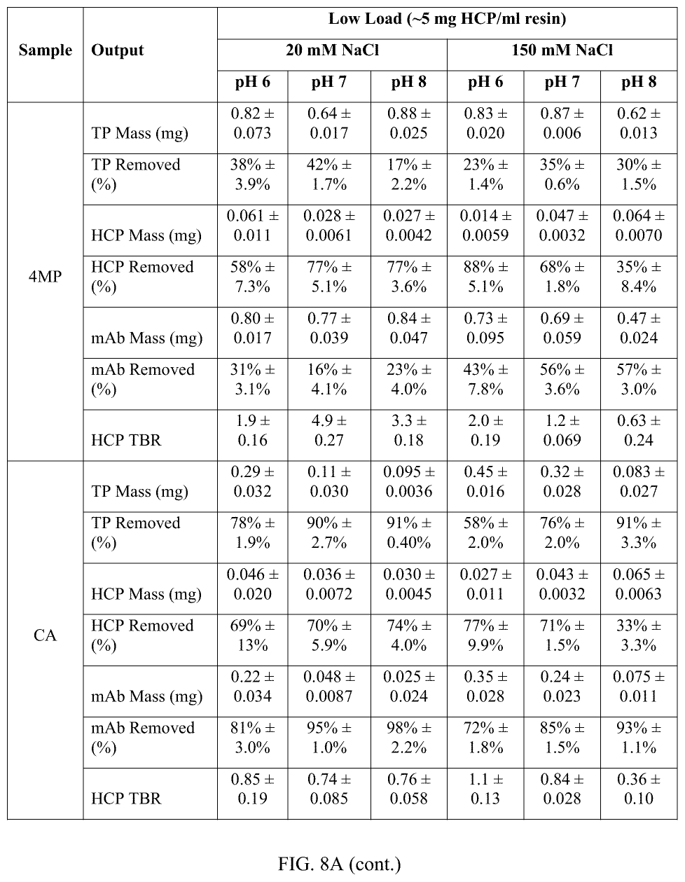

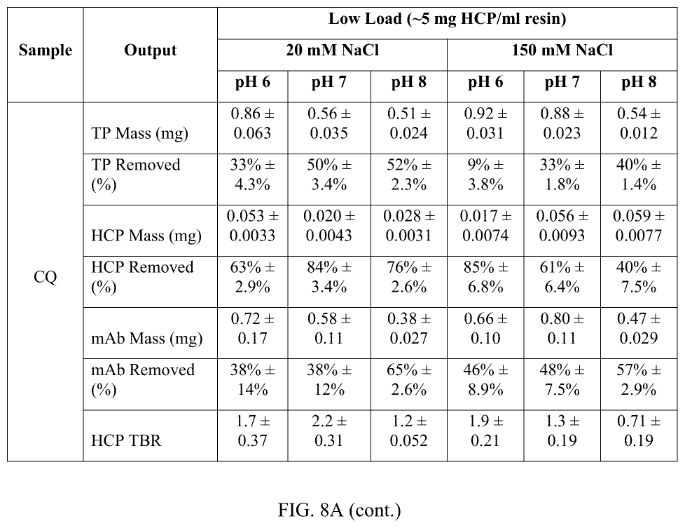

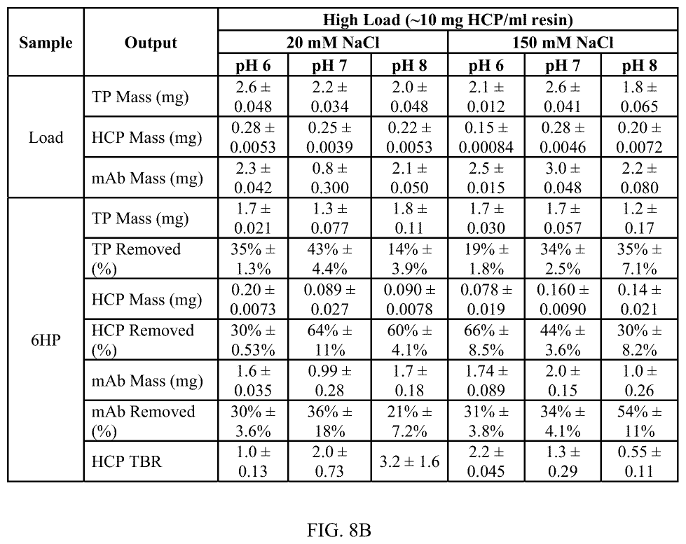

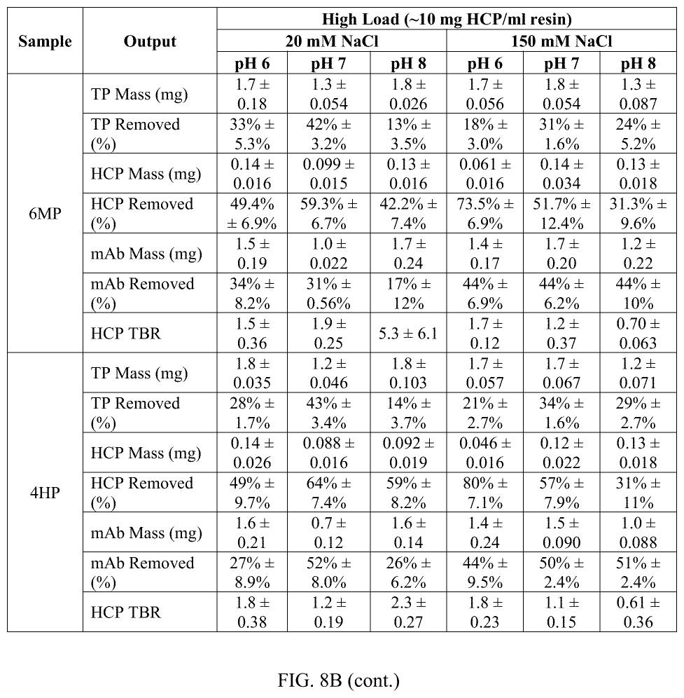

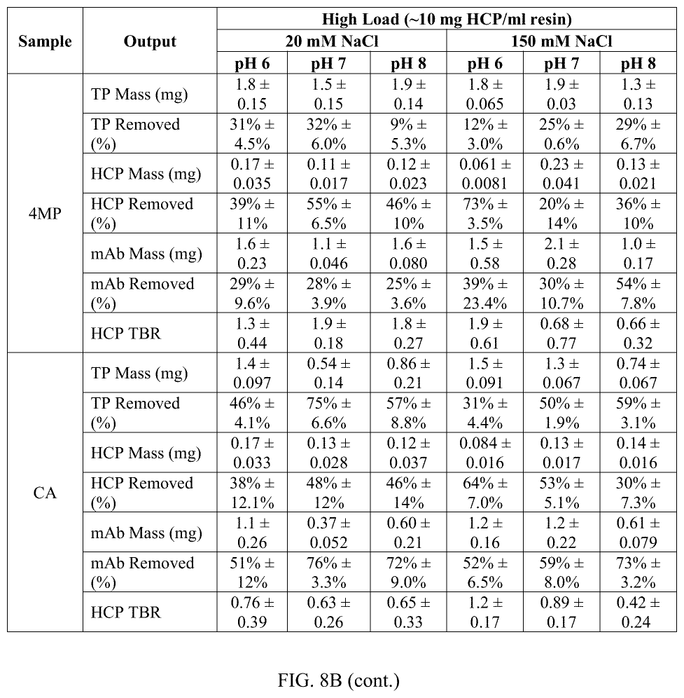

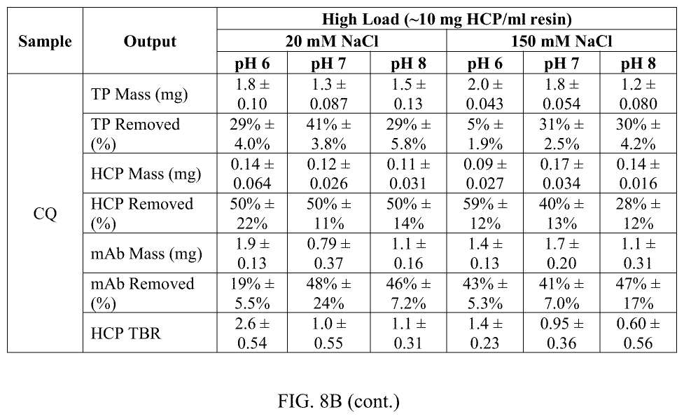

Secondary Screening of HCP-Binding Ligand Groups by Static Binding Evaluation: An ensemble of 18 peptides, selected from the groups listed in Table 1, were individually synthesized on Toyopearl Amino-650M resin and mixed into a single heterogeneous adsorbent as follows: (i) 6HP, including sequences GSRYRYGSG (SEQ ID NO: 19), RYYYAIGSG (SEQ ID NO: 20), AAHIYYGSG (SEQ ID NO: 21), IYRIGRGSG (SEQ ID NO: 22), HSKIYKGSG (SEQ ID NO: 23); (ii) 6MP, including sequences ADRYGHGSG (SEQ ID NO: 24), DRIYYYGSG (SEQ ID NO: 25), DKQRIIGSG (SEQ ID NO: 26), RYYDYGGSG (SEQ ID NO: 27), YRIDRYGSG (SEQ ID NO: 28); (iii) 4HP, including HYAIGSG (SEQ ID NO: 29), FRYYGSG (SEQ ID NO: 30), HRRYGSG (SEQ ID NO: 31), RYFFGSG (SEQ ID NO: 32); and (iv) 4MP, including DKSIGSG (SEQ ID NO: 33), DRNIGSG (SEQ ID NO: 34), HYFDGSG (SEQ ID NO: 35), and YRFDGSG (SEQ ID NO: 36). The adsorbents were evaluated to verify binding capacity and selectivity via equilibrium binding studies at different values of pH (6, 7, and 8) and salt concentration (20 mM and 150 mM) of the binding buffer, using a representative IgG-producing CHO-K1 clarified cell culture harvest; commercial resins Capto Adhere (CA) and Capto Q (CQ) were utilized as controls. Percent protein removal for HCP by HCP ELISA, IgG by Easy-Titer assay, and total protein by Bradford assay are presented in FIG. 7A-FIG. 7F (data tabulated in FIG. 8A and FIG. 8B).

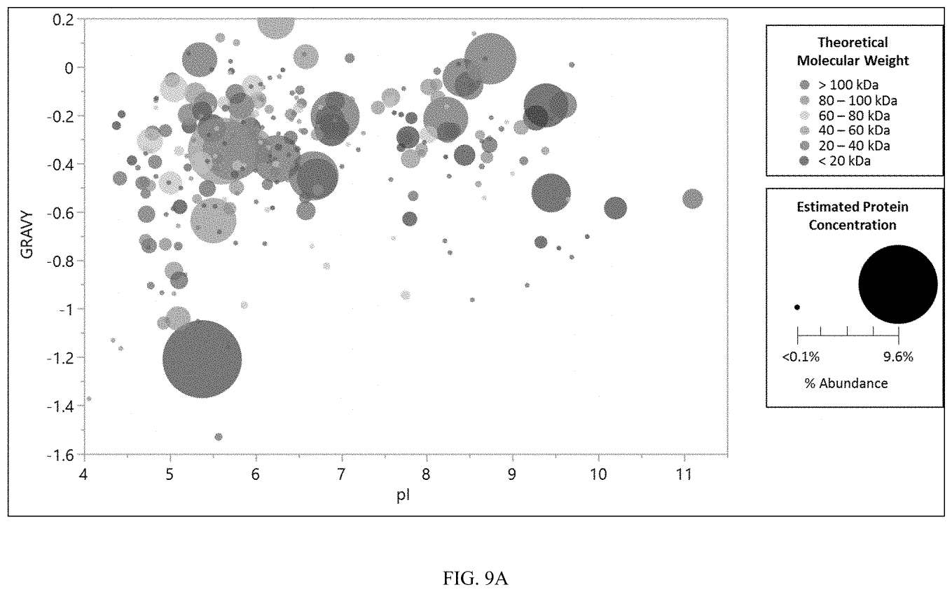

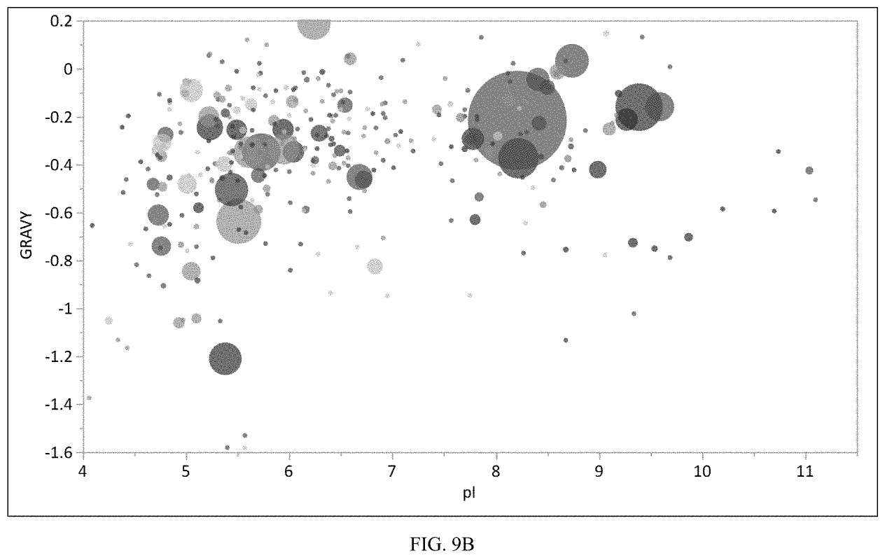

In evaluating protein capture across the four peptide-based adsorbents, consistently higher binding of total protein, host cell protein, and mAb at low salt conditions as compared to high salt conditions was observed, suggesting that, as with Capto Q and Capto Adhere, ionic interactions play a central role in the binding mechanism. The relevance of electrostatic interaction in peptide-HCP binding was anticipated, given that the majority of HCPs have theoretical isoelectric points well below neutral pH (pI<6˜46%, pI<7˜66%, pI<8˜71%, see Table 1 and FIGS. 9A-9B for proteomic composition of the feed stream). Additionally, all species tested in the secondary screening included at least one positively charged amino acid residue and were screen in Bis-Tris or Tris buffer, where the positive buffer ion would interfere minimally with any ionic interactions from positively charged residues.

At the same time, the dependence of total protein (HCP+IgG) binding upon pH varies significantly between Capto Q and the peptide ligands, suggesting that binding on the peptide resins is more multimodal, and potentially sequence-based, in nature than for Capto Q. The differences in mAb binding, in fact, suggest a distinct binding selectivity of the peptides, under the conditions tested, compared to the Capto Adhere multimodal adsorbent. With both MP and HP resins, binding conditions were identified under which observed HCP removal was comparable to the values given by Capto Q and Capto Adhere resins, while percent of mAb loss was equal or lower than that of Capto Q. Moreover, Capto Adhere was found to remove substantially more mAb compared to all other resins, causing a loss of mAb product consistently >70% across all binding conditions. This indicates that the library screening by orthogonal fluorescence method directed peptide selection towards sequences that target HCPs with a degree of affinity higher than mixed-mode level. Interestingly, HCP capture was more robust for the tetrameric ligands as compared to the hexameric ligands in the higher pH regime (pH 7 and pH 8), where as much as 40% more HCP was captured by the tetrameric ligands than the corresponding hexameric peptides. This effect is arguably the result of higher binding selectivity displayed by peptide ligands with longer sequences, which narrows the interaction range to fewer HCP species.

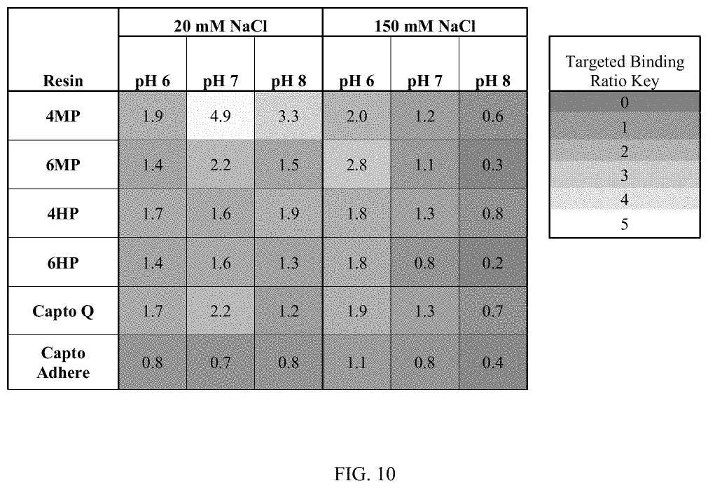

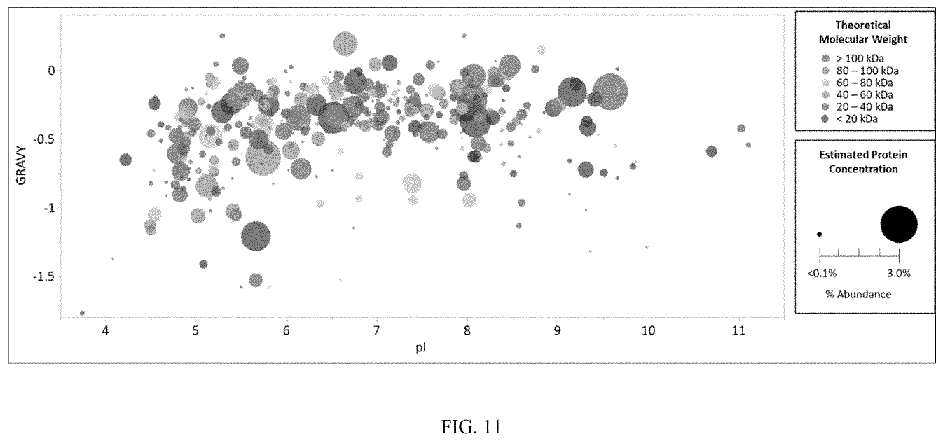

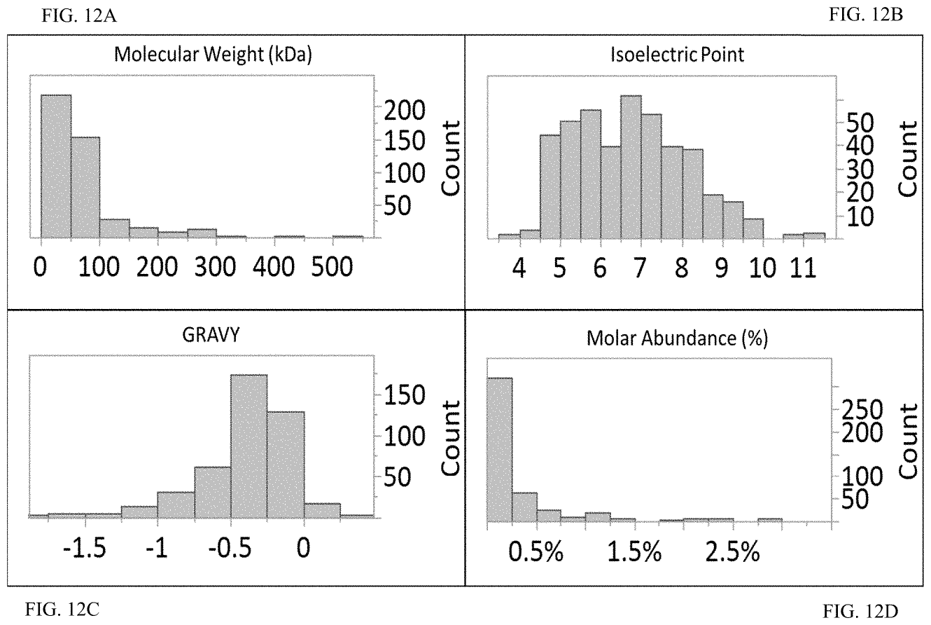

As expected, reduced percent removal was observed with increased protein load across all tested adsorbents, which helped to identify the range in which HCP binding is observable under static binding conditions. As both load conditions were incubated for sufficient time to allow binding equilibrium, screening was conducted at a range of load conditions to ensure that the fraction of HCPs captured was measurable in the static binding supernatant. To recapitulate the specificity of the peptide ligands, the peptide adsorbents were ranked by HCP targeted binding ratio (TBR), herein defined as ratio of host cell protein removed and amount of mAb lost, wherein HCP TBR<1 indicates preferential binding to mAb, and HCP TBR>1 indicates preferential binding to CHO HCPs. The values of HCP TBR by resin and buffer condition are summarized for the low load condition (5 mg/ml) in FIG. 10. Preferential HCP binding by all four peptide adsorbents was observed with most of the binding buffers tested, with the exception of the pH 8, 150 mM NaCl condition. Given that the mAb concentration in the cell culture harvest is at minimum two orders of magnitude higher than any single host cell protein species, as measured in the clarified harvest, the identified peptides must possess a much stronger binding for HCPs compared to mAb. The preferential binding to IgG observed with peptide resins and Capto Q at the pH 8, 150 mM condition, in addition to lower HCP TBR observed at pH 7, 150 mM, was likely a result of buffer pH conditions close to or above the isoelectric point of the mAb (measured at ˜7.6) coupled with higher salt concentration, which minimized the contribution of ionic interactions to binding.

Multipolar peptides showed a superior specificity for HCPs, proving to be valuable alternatives to current mixed-mode ligands for mAb polishing. In particular, the tetrameric 4MP resin offered the highest level (4.868) of HCP TBR at 4.87 at pH 7, 20 mM NaCl, more than double compared to the value afforded by commercial Capto Q (2.226). This result was somewhat unexpected, given the lack of multipolar adsorbents used in the context of biopharmaceutical purification in the art. Without wishing to be bound to a particular theory, it is possible that the mechanism of binding for the multipolar ligands that is quite similar to the double ion pairing mechanisms proposed in enantio- and stereoisomer selective multipolar ligands, wherein strong ionic interaction with the positively charged amino acid on the ligand is paired with a weaker ionic interaction with the negatively charged residue in order for the protein target to remain bound. This mechanism could also be applicable to the hydrophobic/positive ligands, in addition to other commercial multimodal resins such as Capto Adhere, with the exception that the double-ion pairing interaction mechanism is replaced by other binding mechanisms (π-π bonding, Van der Waals interaction, hydrogen bonding, etc.). Should the proposed binding mechanisms proposed be confirmed, the combination of these ligands into a “polyclonal” ensemble would allow for capture of a more diverse set of HCPs than each set alone.

### Example 2

### Capture of Particular HCP Species by Peptide Ligands by Proteomic Analysis

Using the same procedures as exemplified and described in Examples 1 and 2 but using a different method for the relative quantification of individual HCP, the role of various binding buffers was further evaluated.

Relative Quantification of Individual HCPs Using Method 2: Relative quantity of each protein across samples was calculated based on the spectral count (SpC) for each protein (Cooper et al., 2010) in individual samples multiplied by the sample volume. The spectral abundance factor (SAF) of individual proteins in the collected supernatant samples (combination of the unbound fraction from the static binding and the following wash) was calculated as shown in the equation below.

$${SAF}_{i , j} = \frac{S p {C}_{i , j} \times D {F}_{j}}{{L}_{i}}$$

Calculated Spectral Abundance Factor, where: SAF_{i,j}=spectral abundance factor for protein i in sample j (kDa⁻¹), SpCᵢ=spectral count of protein i in sample j, DFⱼ=Dilution factor for sample j, Lᵢ=length of protein i (kDa).

The relative abundance of every HCP in the feed sample was calculated based on normalized spectral abundance factor (NSAF) (Neilson et al., 2013) for each identified protein as shown in the equation below.

$${NSAF}_{i} = \frac{{SAF}_{i}}{\sum SAF}$$

A comparison of the relative quantities of individual HCPs in the supernatant vs. feed samples was conducted by Analysis of Variance (ANOVA) of the SAF for every protein in the corresponding samples using JMP Pro 14. For the analysis of bound HCPs, the SAF values were used to compare the residual amounts of every HCP in the supernatants obtained by static binding of their corresponding feed samples. “Bound HCPs” are herein defined as the proteins that (i) were identified in the majority of feed samples (i.e., had a sum of spectral count greater than 4 across all replicates, N=3) and (ii) were either not found in the supernatant samples or showed significantly lower spectral count (p<0.05 by ANOVA) compared to the feed sample. Venn diagrams of bound proteins across peptide-based and benchmark resins were constructed using the Venn Diagram add-in for JMP Pro 14. The non-normal distributions for isoelectric points of depleted proteins were compared by Kruskal-Wallis H test with a 90% confidence interval using JMP Pro 14.

Analysis of HCP Binding. The CHO HCP-targeting peptide ligands discovered in prior work by screening tetrameric (X₁X₂X₃X₄GSG) and hexameric (X₁X₂X₃X₄X₅X₆GSG) peptide libraries comprise multipolar (MP) and hydrophobic/positive (HP) peptides (Lavoie et al., 2019). MP ligands include sequences with one positively charged (Arg, His, Lys) and one negatively charged (Asp) amino acid residue, with the remaining combinatorial positions filled with aliphatic or aromatic residues. HP ligands include sequences containing one or two positively charged residue(s), with the remainder primarily aromatic residues. The initial characterization of these peptide-based adsorbents led to the identification of buffer conditions that maximize binding specificity for CHO HCPs over the IgG product (Example 2). To that end, the peptide-based resins were compared to commercial resins Capto Q, a strong anion exchange resin featuring a quaternary amine ligand, and Capto Adhere, a mixed-mode resin featuring a combination of strong anion exchange, hydrogen bonding, and hydrophobic functionalities. The binding studies were conducted in static binding mode using a set of different binding buffers (NaCl concentration of 20 or 150 mM; pH 6, 7, or 8). The salt concentration and pH of buffers were selected to evaluate the performance of the resins at “harvest-like” conditions (150 mM NaCl) and “conventional polishing” conditions (20 mM NaCl). The pH range was limited to 6-8 to prevent protein instability in the clarified harvest. The feed samples were prepared by diafiltration of the cell culture fluid against the different buffers, incubated for 1 hour with the equilibrated adsorbents, and the supernatants (unbound and wash fraction) were collected and pooled prior to analysis. The majority of the resins yielded the best selectivity at 20 mM NaCl, pH 7; based on global quantification of HCPs by ELISA, it was found that MP resins had equivalent or increased selectivity for HCPs compared to Capto Q and Capto Adhere (Lavoie et al., 2019). HP resins, while slightly less selective than Capto Q, still exhibited preferential binding to HCPs and were found to be superior to Capto Adhere under the near-neutral pH conditions tested. The peptide-based resins also proved more effective than commercial resins in HCP binding studies performed at “harvest-like” condition (150 mM NaCl), suggesting potential use as pre-Protein A HCP scrubbers. These conditions were not specifically optimized for flow-through operation of commercial resins; Capto Q is in fact normally operated at low salt conditions, whereas Capto Adhere is utilized at fairly low pH values to prevent binding of the mAb product. The scope of this work, however, is to directly compare peptide-based and commercial resins under equivalent buffer conditions to highlight the ability of peptide ligands to capture HCPs efficiently and selectively without requiring the level of process optimization.

In this study, the HCPs in the supernatant samples from the static binding experiments were identified and quantified via bottom-up, label-free proteomics, and the resulting values were used to evaluate differences in binding of the various HCP groups by the peptide-based resins in comparison with the benchmark commercial resins. In this work, a “bound HCP” was defined as a protein that (i) is detected in the feed stream by LC/MS/MS analysis and (ii) is either not detected in the supernatant (unbound+wash) or has a significantly lower SAF compared to the feed sample (p<0.05 by ANOVA).

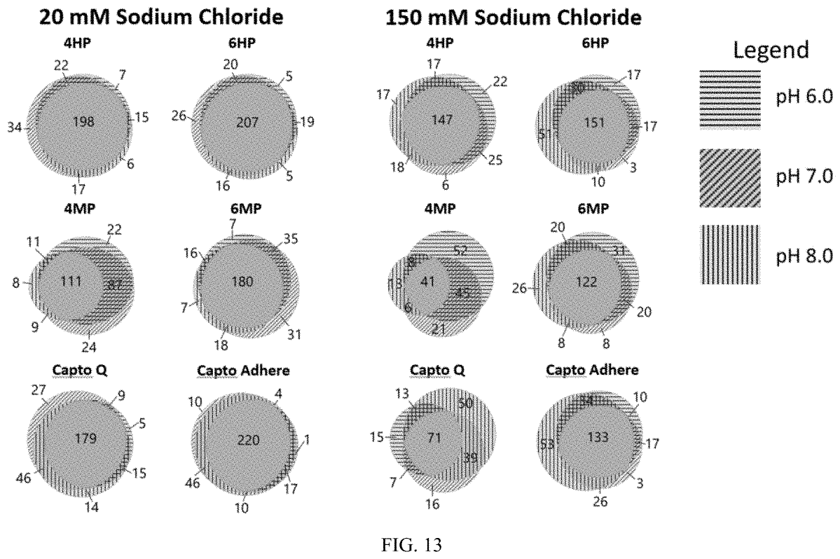

Profile of Bound HCPs vs. pH of the Binding Buffer. The number of unique HCPs bound by the peptide-based and the commercial benchmark resins at different pH conditions are presented in FIG. 13. The analysis of overlapping bound HCPs for the various resins as a function of buffer condition indicates that both 4HP and 6HP resins feature a higher tolerance to differences in pH compared to the benchmark and MP resins, at both salt concentrations (20 mM and 150 mM). As shown in FIG. 13, of all unique bound HCPs across the three values of pH, 4HP and 6HP respectively bound 66.2% (198 of 299 unique proteins) and 69.4% (207 of 298) at 20 mM NaCl, while 58.3% (147 of 199) and 54.1% (151 of 279) at 150 mM NaCl. In comparison, benchmark anion exchange resin Capto Q yielded 60.7% (179 of 295) at 20 mM and 33.6% (71 of 211) at 150 mM. Lower HCPs binding by Capto Q at high salt concentration was anticipated, given that this resin relies solely on electrostatic binding; further, significant capture of the mAb product (isoelectric point˜7.6) by Capto Q at pH 8 also reduces the number of binding sites available for HCP capture (Lavoie et al., 2019). The mixed-mode resin Capto Adhere showed a high overlap in bound HCPs (71.4%, 220 of 308) at low salt concentration; however, promiscuous binding of HCPs was also accompanied by significant loss (>80% for all pH conditions) of mAb product (Lavoie et al., 2019). The analysis of protein binding at 150 mM NaCl showed a decrease in overlap of bound HCPs to 48.2% (133 of 276 bound proteins), indicating poor tolerance to pH variations. The ability of HP resins to maintain HCP binding almost constant under different pH conditions shows that the peptide ligands feature a stronger affinity-like binding activity than commercial mixed-mode ligands, which often require extensive optimization of the process conditions to grant sufficient product yield and purity. Robustness in HCP capture within a design space of buffer conditions by peptide ligands makes them more apt towards platform processes for mAb purification.

Turning to multipolar ligands, 4MP and 6MP resins showed rather conspicuous differences in HCP binding. The 6MP resin compared well with its HP counterparts in terms of robustness of HCP capture against different pH conditions, with overlaps of bound HCPs of 61.2% (180 of 294) and 51.9% (122 of 235) at 20 mM and 150 mM, respectively. The 4MP ligand, on the other hand, demonstrated poor tolerance to pH differences at both 20 mM and 150 mM NaCl, with overlaps of bound HCPs of 40.8% (111 of 272) and 22.0% (41 of 186), respectively. A unique feature of the 4MP resin was its inverse relationship between HCP binding and buffer pH. As the net charge of the proteins in solution is shifted towards negative values as the pH of the binding buffer increases, the presence of negatively charged amino acids in the 4MP peptide ligands explains the loss of HCP binding at higher pH.

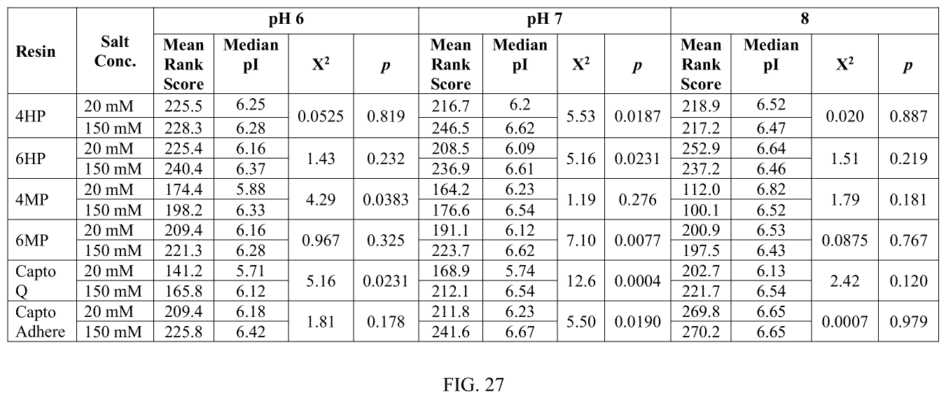

A comparison of the distributions of pI values among the HCPs bound at different pH conditions was also performed using the Kruskal-Wallis H test to evaluate the shift in the charge profile of the HCPs in the supernatant vs. feed samples. The Kruskal-Wallis H test, as shown in the table in FIG. 27, was adopted given the non-normal distribution of the pI values. If HCP binding by the peptide-based resin were dominated by electrostatic interactions, the pI profiles of bound HCPs would differ significantly among different pH conditions; in particular, the median pI would be expected to increase at higher binding pH, as HCPs with higher pI values would become negatively charged and be captured by the positively charged HP ligands. Notably, no significant shift in the isoelectric point profile of bound proteins was observed for the 4HP resin (p=0.171 and p=0.355 for 20 mM and 150 mM NaCl, respectively), whereas the 6HP resin showed a statistically significant shift only for the 150 mM NaCl condition (p=0.392 and p=0.0086 for 20 mM and 150 mM NaCl, respectively). This indicates that HCP:peptide interactions for 4HP and 6HP are not entirely dependent on electrostatic interaction; for comparison, the traditional anion exchange resin Capto Q shows a significant increase in pI as a function of pH at both salt conditions (p=0.0969 at 20 mM and p=0.0434 at 150 mM). Capto Adhere, whose ligand (whose 2-benzyl,2-hydroxyethyl,2methyl-ammonioethyl) has a strong similarity with the HP peptides, showed non-significant response in terms of pI distribution of bound HCPs vs. pH at low salt and (p=0.240 at 20 mM), but significant at high salt (p=0.0130 at 150 mM). With multipolar ligands, a significant correlation between pH of binding and pI profile of bound HCPs was observed only with the 4MP resin at the high salt condition (p=0.0028). The presence of both positive and negatively charged residues on MP ligands makes their interaction with HCPs more complex; the softening of electrostatic repulsions at high ionic strength allows the 4MP ligands to behave more similarly to conventional ion exchangers. Collectively, this indicates a stronger correlation between binding pH and pI profile of bound HCPs at higher ionic strength of the binding buffer (150 mM vs. 20 NaCl, FIG. 27). This result occurs not only from a shift in HCP:peptide binding strength at different salt concentrations (Tsumoto et al., 2007), but also from a decrease in non-specific adsorption of the highly abundant mAb product, which furthers the availability of binding sites for HCP capture.

TABLE 3
Kruskal-Wallis H test for bound protein isoelectric
point as a function of buffer pH

| | | 20 mM NaCl | | | | 150 mM NaCl | | | |
|---|---|---|---|---|---|---|---|---|---|
| | | Mean | | | | Mean | | | |
| | | Rank | Median | | | Rank | Median | | |
| Resin | pH | Score | pI | X² | p | Score | pI | X² | p |
| 4HP | 6 | 357.7 | 6.28 | 3.53 | 0.171 | 300.0 | 6.25 | 2.07 | 0.355 |
| | 7 | 393.3 | 6.62 | | | 293.0 | 6.2 | | |
| | 8 | 371.7 | 6.47 | | | 317.5 | 6.52 | | |
| 6HP | 6 | 372.6 | 6.37 | 1.88 | 0.392 | 302.0 | 6.16 | 9.51 | 0.0086 |
| | 7 | 398.4 | 6.61 | | | 301.8 | 6.09 | | |
| | 8 | 379.9 | 6.46 | | | 348.3 | 6.64 | | |
| 4MP | 6 | 292.8 | 6.33 | 0.839 | 0.658 | 148.8 | 5.88 | 11.8 | 0.0028 |
| | 7 | 305.8 | 6.54 | | | 164.1 | 6.23 | | |
| | 8 | 306.6 | 6.52 | | | 196.4 | 6.82 | | |
| 6MP | 6 | 348.5 | 6.28 | 2.79 | 0.248 | 257.4 | 6.16 | 3.89 | 0.143 |
| | 7 | 378.5 | 6.62 | | | 251.8 | 6.12 | | |
| | 8 | 356.8 | 6.43 | | | 282.2 | 6.53 | | |
| Capto | 6 | 335.9 | 6.12 | 4.67 | 0.0969 | 189.8 | 5.71 | 6.28 | 0.0434 |
| Q | 7 | 375.4 | 6.54 | | | 197.9 | 5.74 | | |
| | 8 | 369.7 | 6.54 | | | 223.4 | 6.13 | | |
| Capto | 6 | 386.6 | 6.42 | 2.858 | 0.240 | 290.2 | 6.18 | 8.69 | 0.0130 |
| Adhere | 7 | 417.5 | 6.67 | | | 295.8 | 6.23 | | |
| | 8 | 416.6 | 6.65 | | | 335.9 | 6.65 | | |

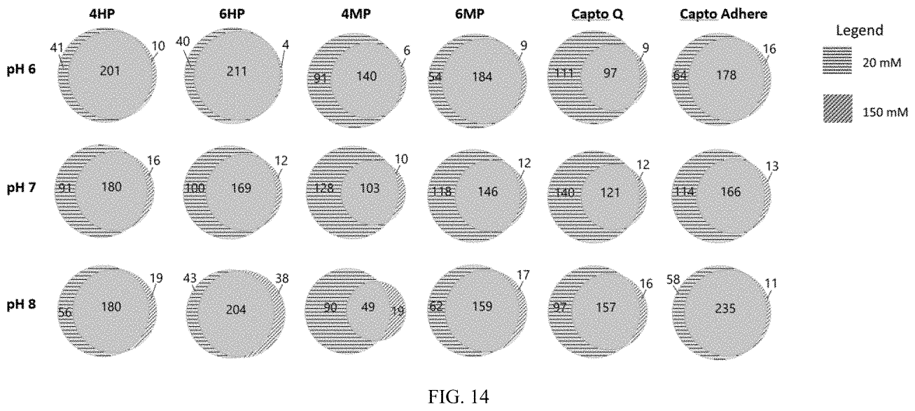

Profile of Bound Proteins vs. Ionic Strength of the Binding Buffer. Overlap in bound HCPs as a function of ionic strength was additionally assessed to compare the tolerance of the different ligands to salt concentration. The comparison of HCP binding at 20 mM vs. 150 mM NaCl concentration is reported in FIG. 14 for all resins and binding pH. Notably, the proteomic analysis of the supernatant samples obtained with peptide-based resins showed a strong tolerance to 150 mM, a typical salt concentration in clarified cell culture harvests. When tested at 150 mM NaCl, in fact, 4HP and 6HP ligands in particular maintained the binding of a significant fraction of HCPs (60.1-82.7%) demonstrated at 20 mM NaCl. As anticipated for an ion exchange resin, Capto Q showed a significant reduction in the number of HCPs bound as the salt concentration increased, and consequently a decrease in the number of overlapping bound proteins. Percent overlapping of bound HCPs by Capto Adhere was closer to the values obtained with HP resins (69.0%-77.3%), but was also associated with significantly higher binding of the mAb product, as shown in Example 2. Multipolar resins 4MP and 6MP showed substantially different binding behavior as a function of salt concentration. Good salt tolerance, comparable to that of HP resins, was observed with 6MP resin, which provided an overlap in bound HCPs of 52.9%-66.8%. On the contrary, 4MP resin showed low tolerance to salt concentration, similarly to what observed in response to pH conditions.

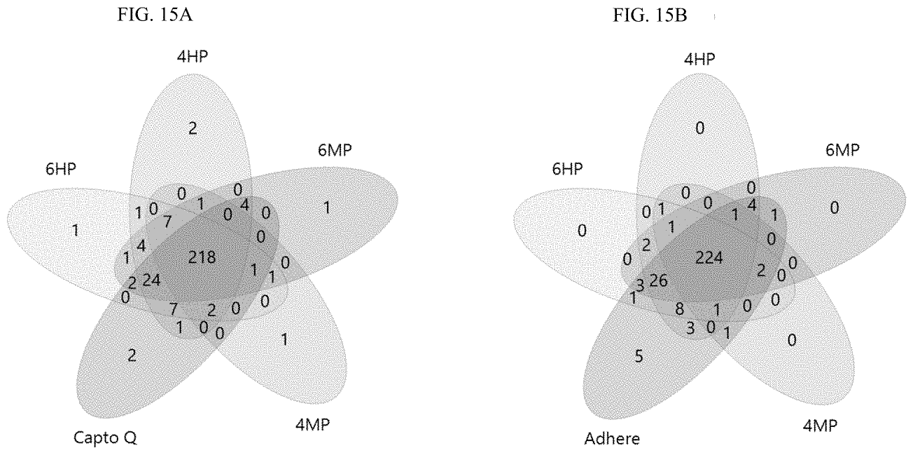

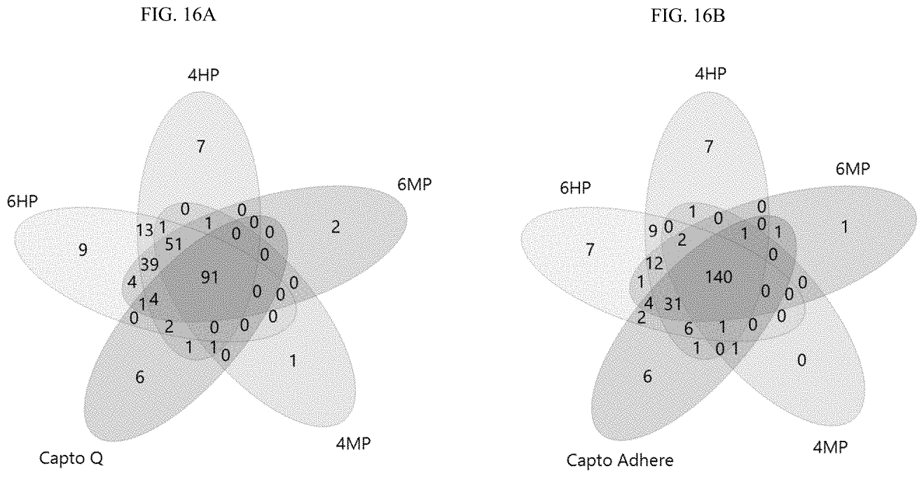

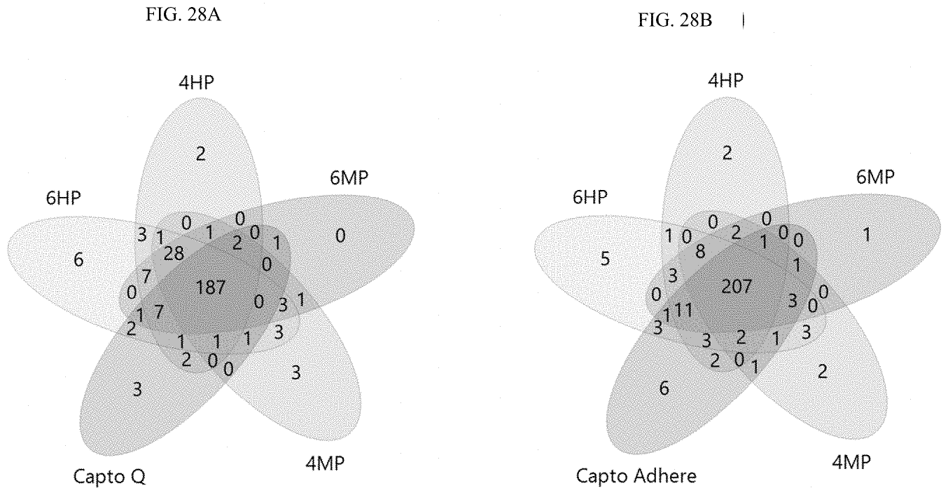

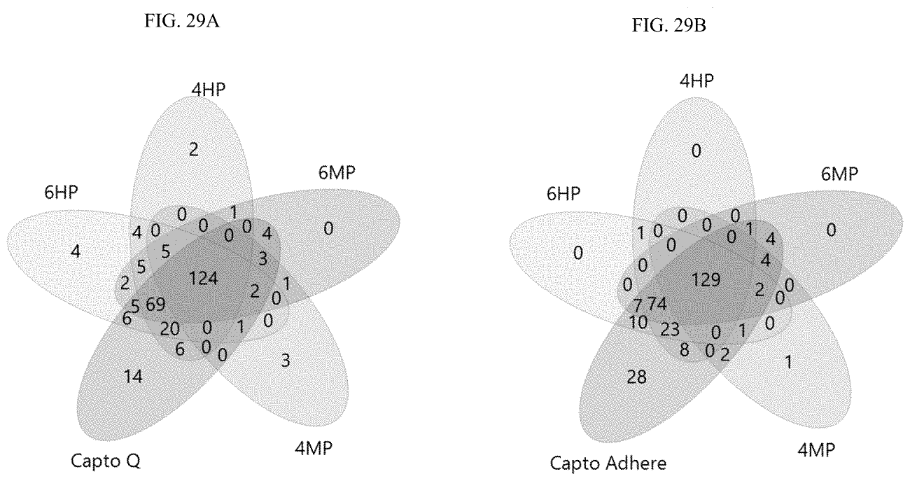

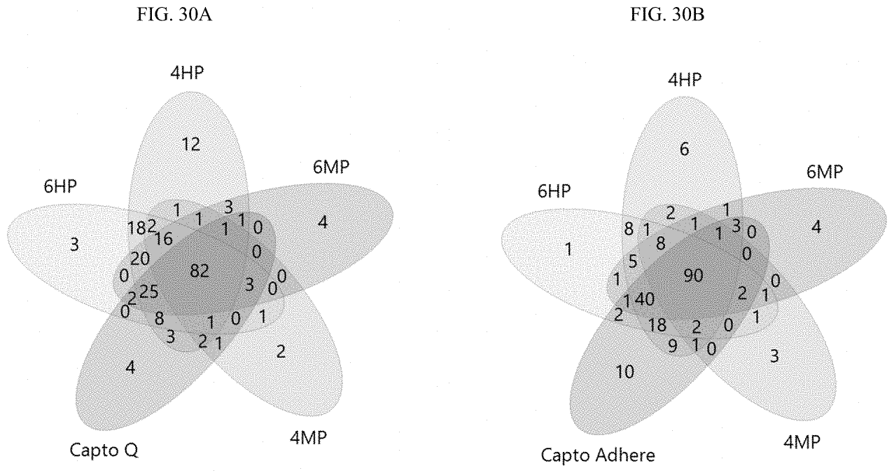

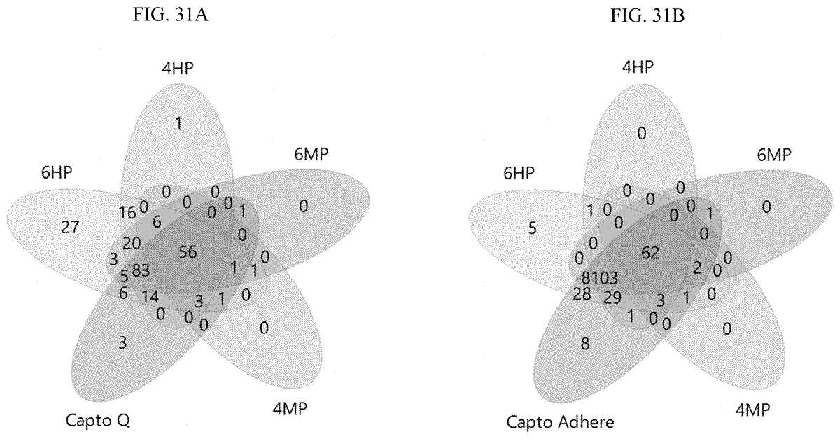

Profile of Bound Proteins by Peptide-based Resins vs. Commercial Resins. A comparison of the HCP species bound by the various resins at given binding conditions (pH and salt concentration) was then performed to identify proteins uniquely bound by a single or a set of resins. Our analysis focused on the optimal binding conditions identified in prior work (Lavoie et al., 2019), namely pH 7 at 20 mM NaCl and pH 6 at 150 mM NaCl, whose results of overlap of protein binding by the various resins are presented as Venn diagrams in FIG. 15A and FIG. 15B and FIG. 16A and FIG. 16B. Analogous plots for the other binding conditions are available in FIGS. 28-31

Proteomic analysis of the fractions generated at 20 mM NaCl, pH 7 indicates substantial overlap in unique proteins bound between the peptide resins and the benchmark resins. Capto Q, in particular, afforded significant binding of 261 unique proteins, of which only 2 were not bound by any of the peptide resins, namely EF-HAND 2 containing protein and fatty acid-binding protein (adipocyte), neither of which has been reported as a problematic HCP to our knowledge. On the other hand, peptide resins showed significant binding of additional 20 unique HCP species, including problematic HCPs from Group I (peptidyl-prolyl cis-trans isomerase, fructose-bisphosphate aldolase, sulfated glycoprotein 1, glyceraldehyde 3-phosphate dehydrogenase, and biglycan). From the perspective of overall product purity, Group I Protein A co-eluting HCPs are the most challenging to address, as a large majority of these proteins are indicated to co-elute as a result of association to the product (Aboulaich et al., 2014; Levy et al., 2014) or association to histones that can in turn non-specifically bind to multiple entities (Mechetner et al., 2011). The efficient capture of product-bound species in this group may explain to some degree the loss of IgG observed in prior work (Lavoie et al., 2019), as some IgG molecules may associate with the HCPs retained by the HP ligand. The HCP retention by the 6HP peptides matched the performance of Capto Adhere, a commercial mixed-mode ligand that possesses a broad and strong HCP binding capacity under these buffer conditions. 6HP showed significant binding of 15 of the 20 additional species, but failed to bind fructose-bisphosphate aldolase, which was captured only by 4MP, in addition to one form of peptidyl-prolyl cis-trans isomerase.

In comparison to the benchmark mixed-mode resin, the peptide resins bound 280 of the 285 unique species bound by Capto Adhere, while also showing a significantly lower binding (>2-fold) of the mAb product. Four HCP species, including problematic HCP sulfated glycoprotein 1, in addition to tenascin-X, copper transport protein ATOX1, and procollagen C-endopeptidase enhancer 1, were captured by one or more peptide-based resins, but did not show binding to Capto Adhere under these conditions. A large majority of the species bound by Capto Adhere (270 of 285) was also captured by the 6HP resin; this was expected, given similarities in the potential binding interactions between the two resins, despite significant differences in mAb product binding.

A parallel analysis of the fractions generated at 150 mM NaCl, pH 6, summarized in FIG. 16, indicates considerable differences in the capture of host cell proteins by the peptide resins vs. the benchmark resins. As shown in FIG. 16A, the peptide resins bound 128 unique proteins in addition to 100 of the 106 proteins bound by Capto Q, including problematic HCPs from Group I (heat shock cognate protein, pyruvate kinase, 60S acidic ribosomal protein P0, elongation factor 2, nidogen-1, elongation factor 1-alpha, cofilin-1, out-at-first protein-like protein, aldose reductase-related protein 2, peroxiredoxin-1, biglycan, glutathione s-transferase, alpha-enolase, and glyceraldehyde-3-phosphate dehydrogenase), Group I/II (cathepsin B, matrix metalloproteinase-9, matrix metalloproteinase-19, protein disulfide-isomerase, serine protease HTRA1), Group I/III (glutathione s-transferase), and Group III (phospholipase B-like protein, procollagen-lysine,2-oxoglutarate 5-dioxygenase 1, and peroxiredoxin-1). A large majority (117 of 128) of the species that do not bind to Capto Q, but do bind to at least one peptide resin showed binding to the 6HP resin. Notable exceptions include peptidyl-prolyl cis-trans isomerase, which was bound by 4HP and both MP resins, as well as biglycan, glutathione s-transferase P, alpha-enolase, and glyceraldehyde-3-phosphate dehydrogenase, which were only bound by 4HP. In comparison, of the 6 HCPs bound exclusively by Capto Q, only one has been reported as a problematic, namely 60S acidic ribosomal protein P2. The overlap of bound HCPs shown in FIG. 16B indicates a broader binding by Capto Adhere compared to Capto Q, as well as a larger group of shared bound proteins between the peptide resins and Capto Adhere. Nonetheless, the peptide resins bound 40 more unique species than Capto Adhere while showing significantly lower mAb product binding.

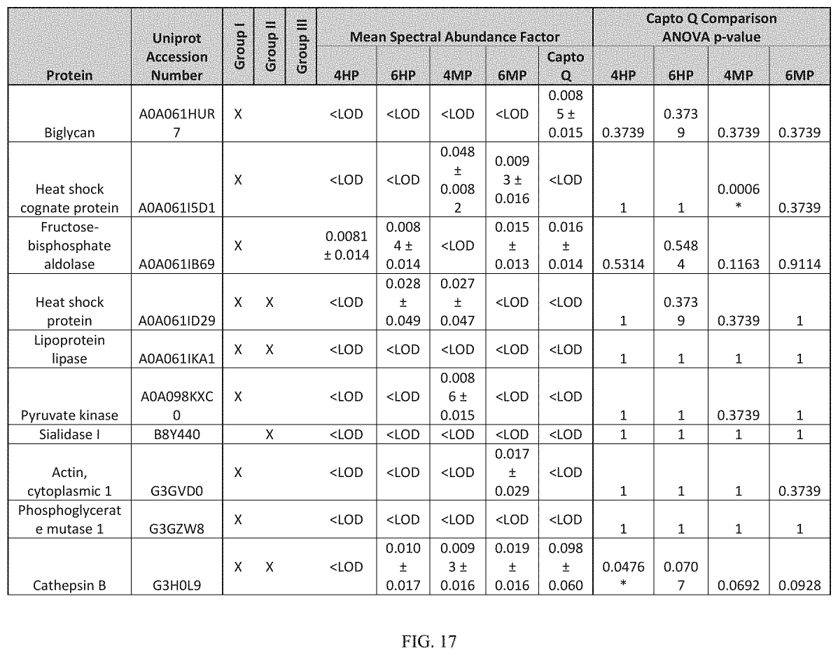

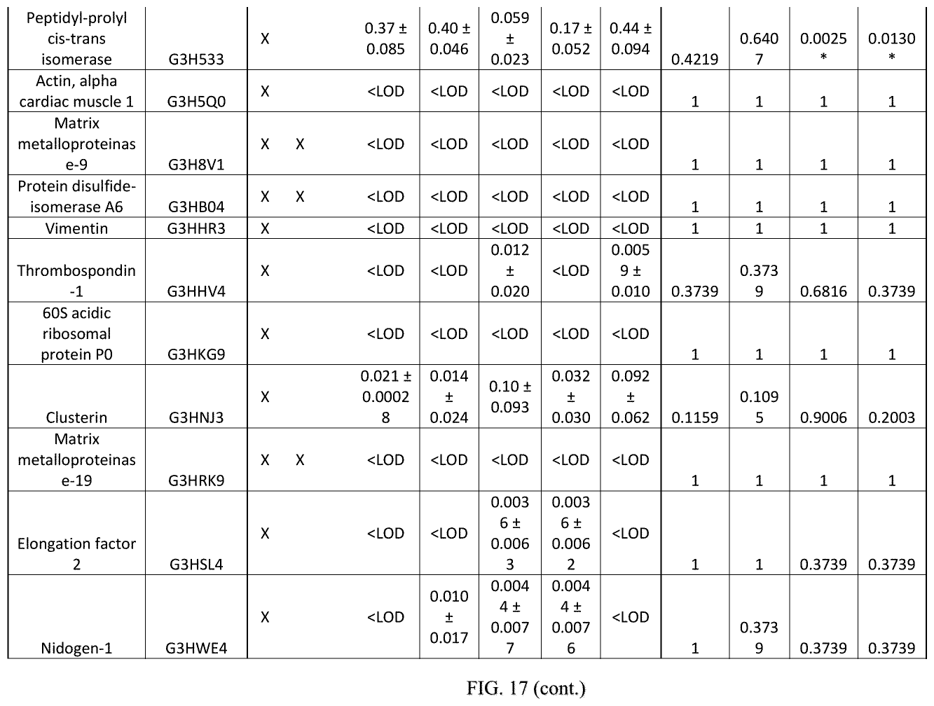

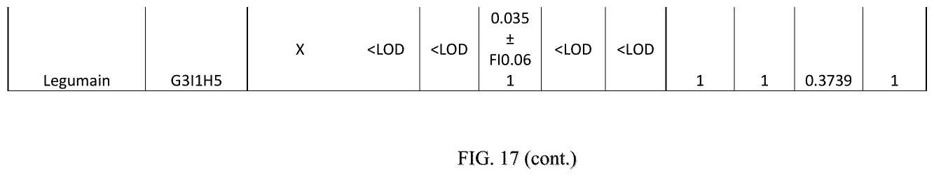

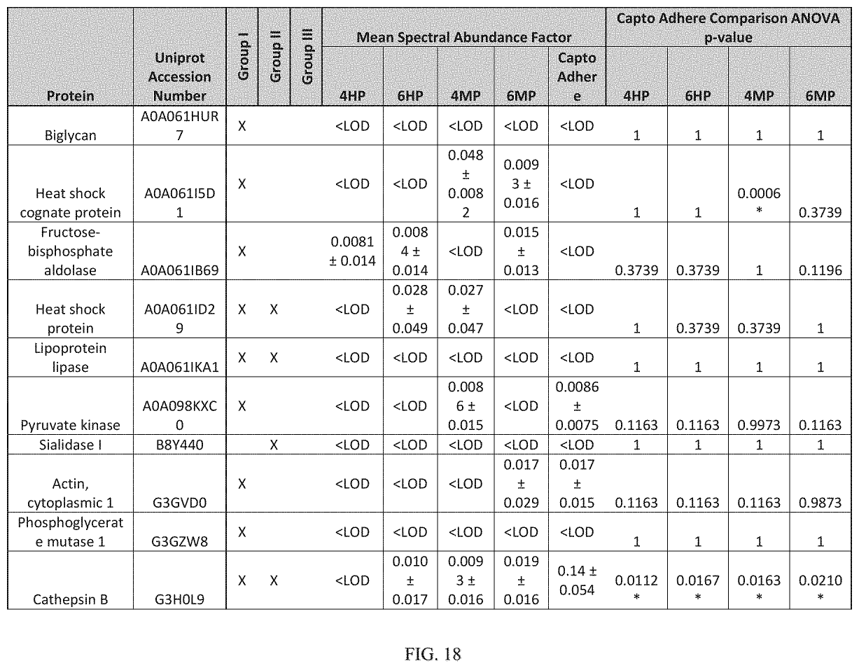

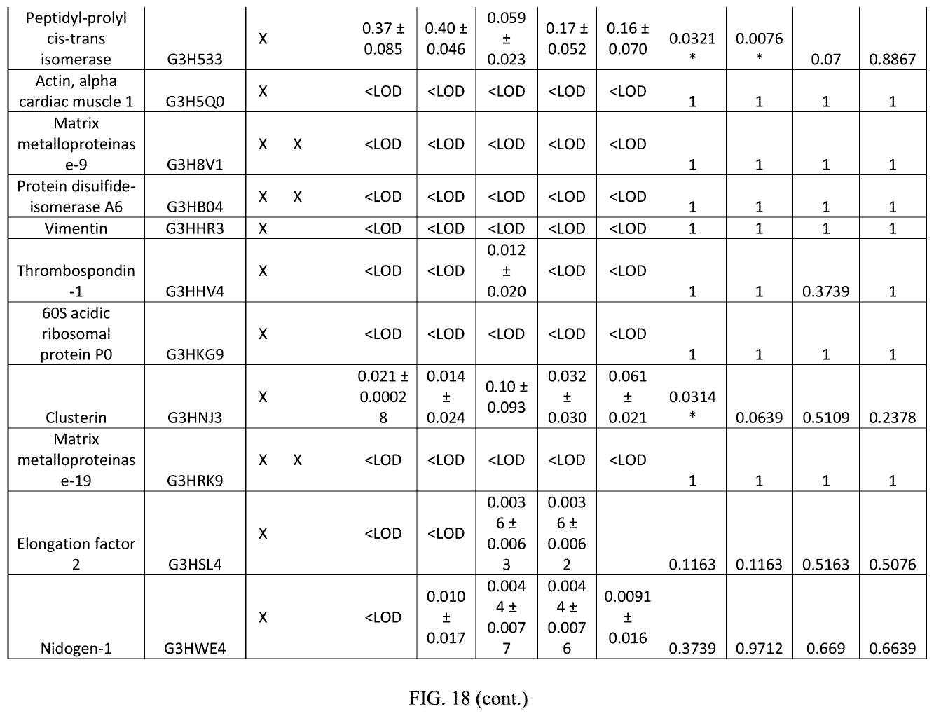

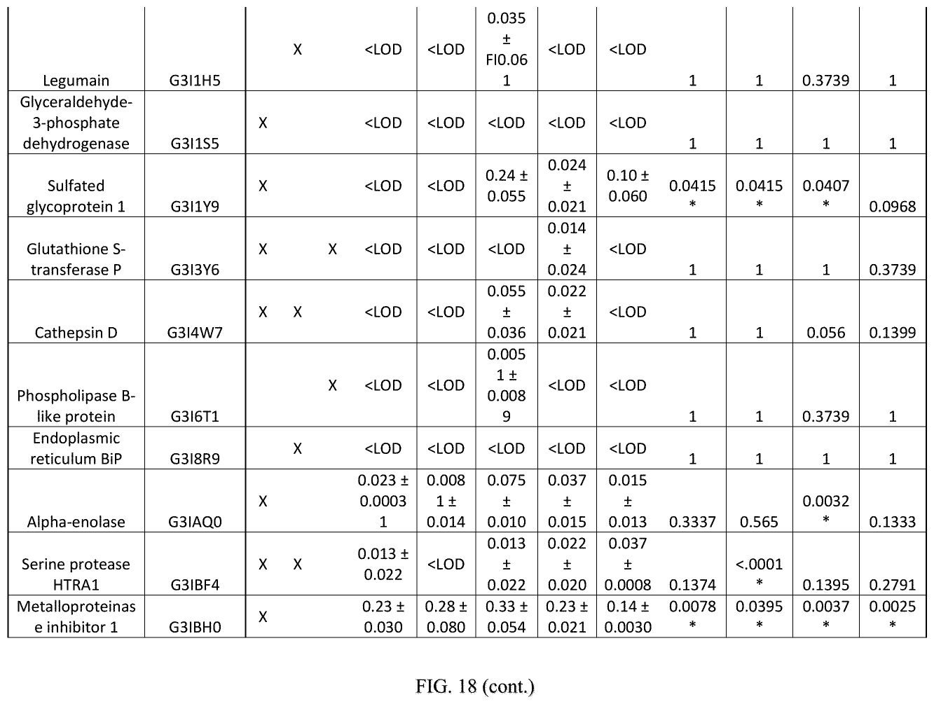

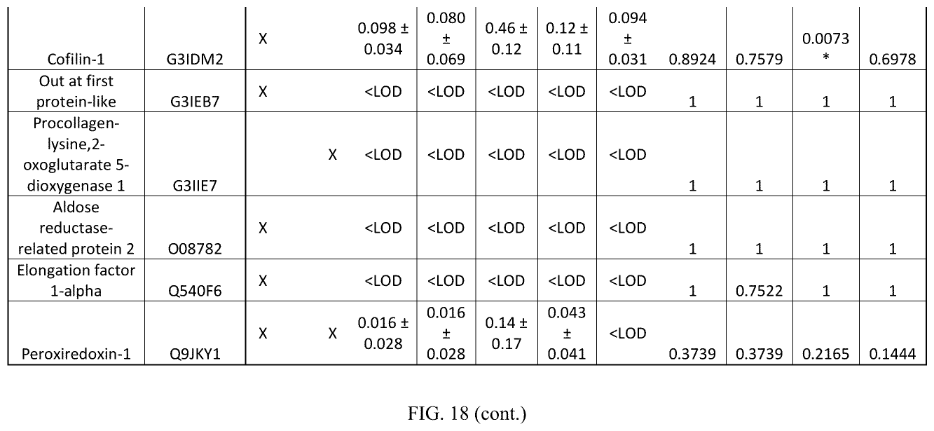

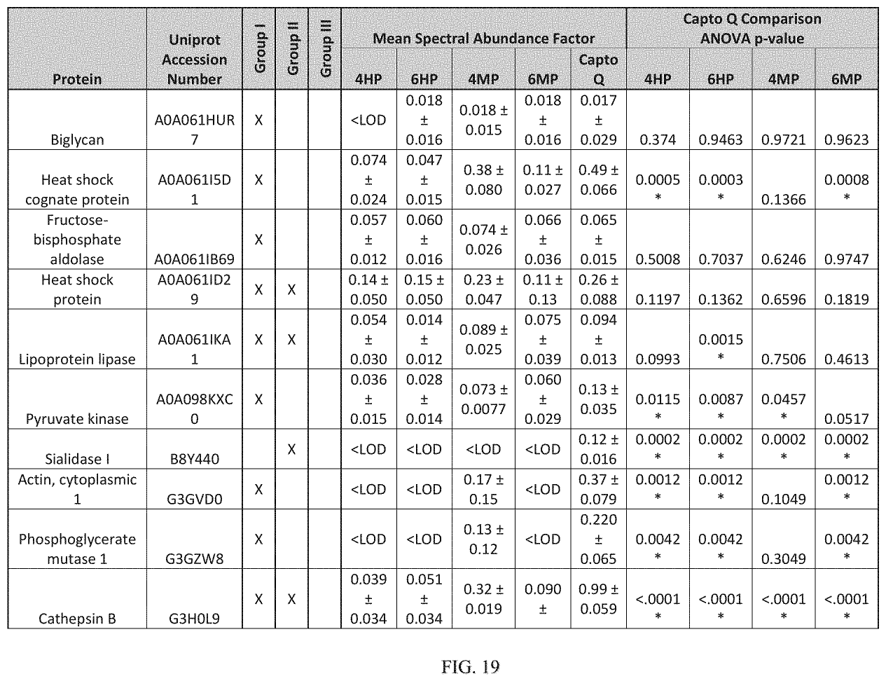

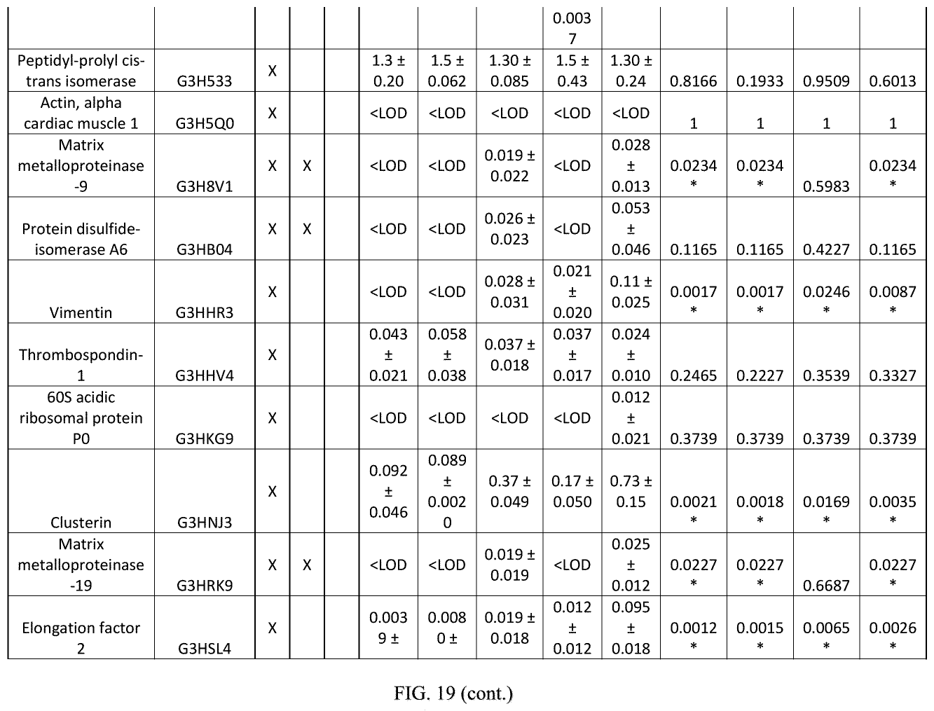

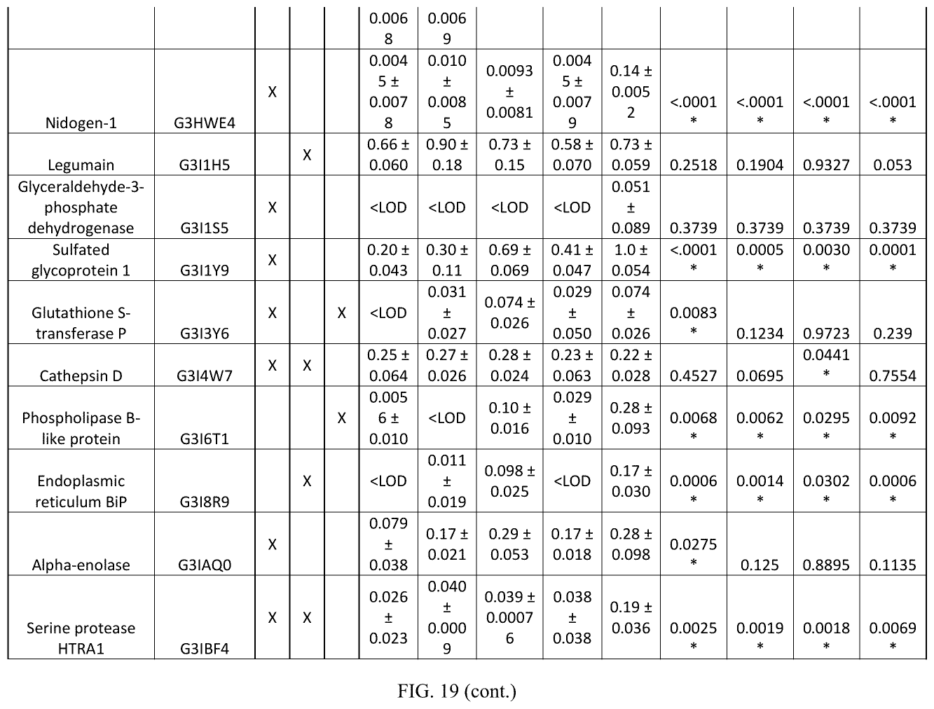

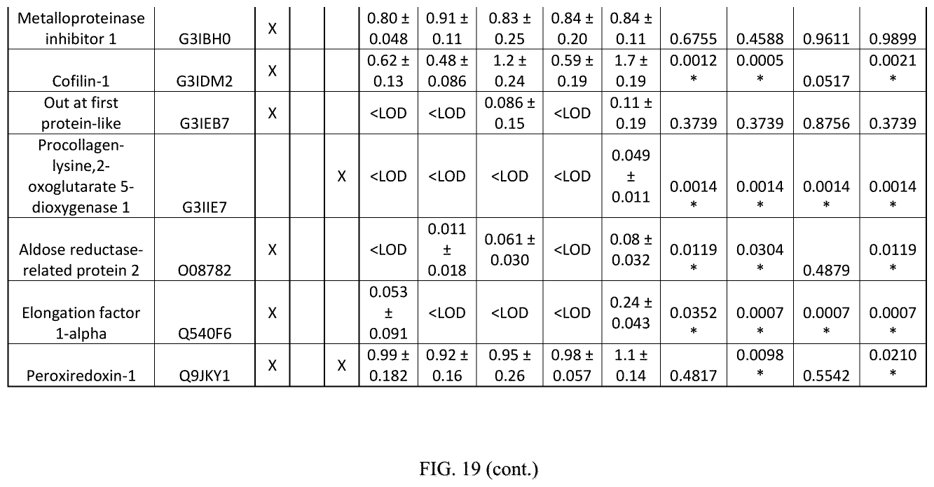

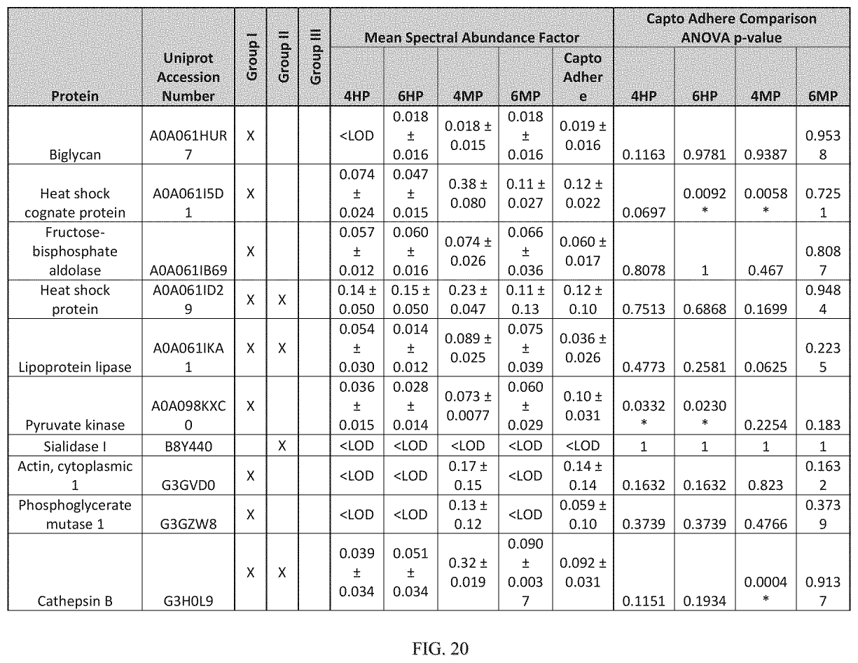

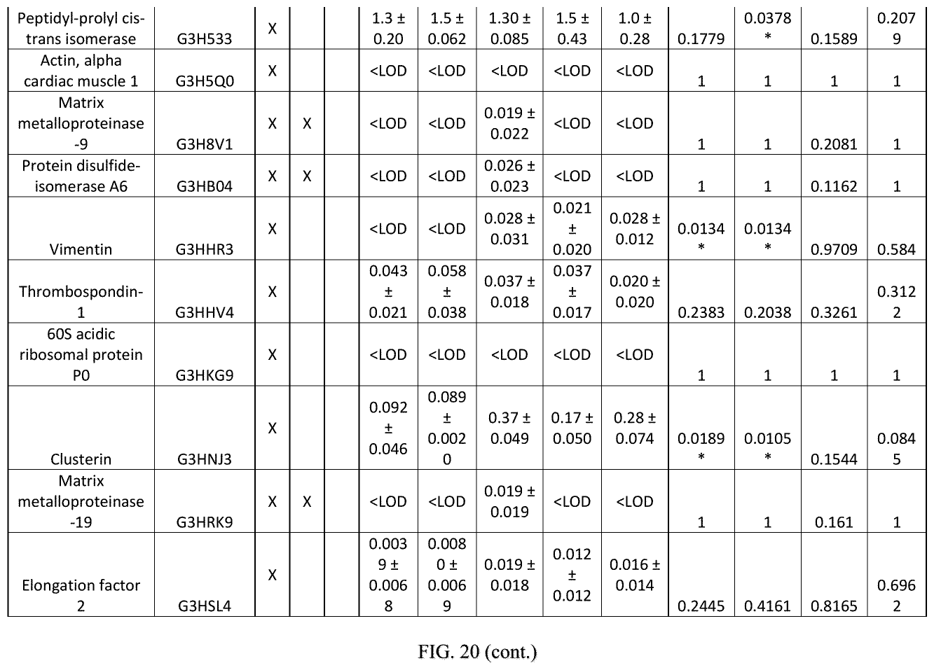

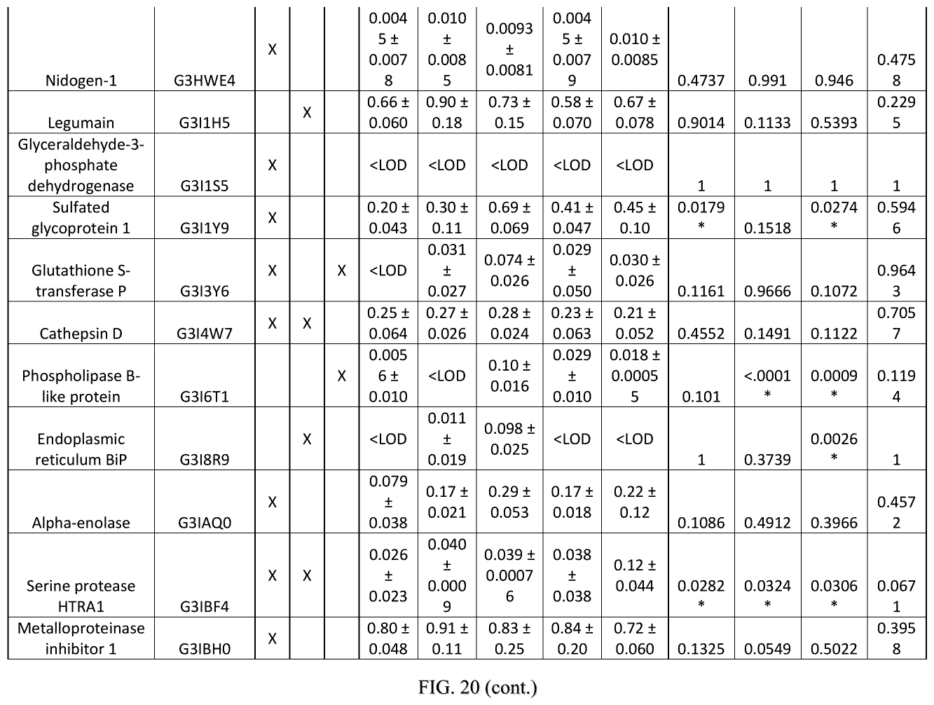

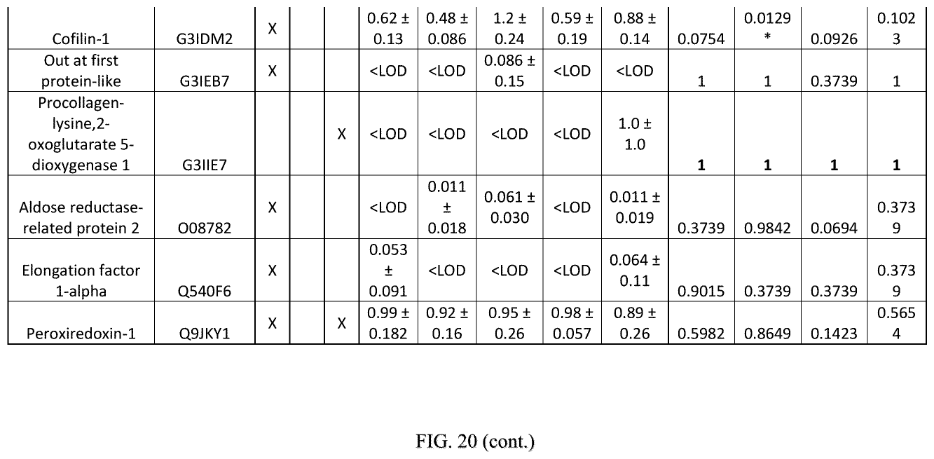

Semi-Quantitative Evaluation of the Binding of “Problematic” HCPs by Peptide Resins vs. Benchmark Resins. To gather a quantitative measure of the differences in HCP-binding activities of the peptide-based resins, label-free relative quantification based on proteomics analysis of the collected fractions was conducted by LC/MS/MS. Specifically, data dependent acquisition (DDA) methods were adopted to compare the relative SAF of every HCPs species in the supernatant samples obtained from static binding tests using the peptide-based resins and benchmark resins Capto Q and Capto Adhere shown in FIG. 17, FIG. 18, FIG. 19, and FIG. 20.

This study was limited to the supernatant samples obtained at the conditions that proved most effective for HCP binding, namely 20 mM NaCl at pH 7, and 150 mM NaCl at pH 6 (Lavoie et al., 2019). The resulting values of SAF for problematic HCP species identified in the supernatants produced at 20 mM NaCl at pH 7 are listed in the table in FIG. 17 and FIG. 18. These values of SAF were compared by an ANOVA (N=3) between the peptide-based resins and both benchmark resins (Capto Q comparison in FIG. 17, and Capto Adhere comparison in FIG. 18) to evaluate the advantage of using peptide ligands for HCP removal. Significantly higher binding was observed for several problematic HCP species by the peptide-based resins compared to Capto Q: cathepsin B, serine protease HTRA1, peptidyl-prolyl cis-trans isomerase, peroxiredoxin-1. 6HP resin was particularly effective compared to Capto Q in binding Group I/II HCP serine protease HTRA1 and Group I/III HCP peroxiredoxin-1, and outperformed Capto Adhere, its small molecule cognate, in binding serine protease HTRA1. 4HP showed improved binding of Group I/II HCP cathepsin B compared to both Capto Adhere and Capto Q. Notably, the binding of peptidyl-prolyl cis-trans isomerase by both MP resins was significantly higher compared to Capto Q and on par with Capto Adhere; it should be noted, however, that the capture of this hard-to-clear species by Capto Adhere comes with a much higher cost in terms of mAb loss compared to MP resins. It was also observed that fructose-bisphosphate aldolase was depleted to levels below the limit of detection by 4MP alone amongst the peptide resins, although the difference in mean spectral counts was not statistically significant, matched only by the higher product binding Capto Adhere.

The development of salt-tolerant stationary phases for mAb purification is much sought after, as they provide flexibility in process implementation. As a result, the binding of HCP species in 150 mM NaCl at pH 6, was analyzed. The values of total HCP clearance and HCP vs. IgG binding determined by ELISA tests indicated that, at this condition, all four peptide-based resins performed equivalently or better than Capto Q (Lavoie et al., 2019).

SAF for HCP species at 150 mM NaCl by both peptide-based and benchmark resins were calculated, as shown in FIG. 19 compared to Capto Q and FIG. 20 compared to Capto Adhere. While increasing salt concentration resulted in an overall reduction in HCP binding, a marked improvement in capture by the peptide ligands was also observed compared to Capto Q. The HP resins were the most versatile in HCP capture, showing significantly higher binding for a large majority of the species in this subset compared to other resins. In particular, 4HP showed significantly lower spectral abundance (higher binding) for 21 of the 37 problematic HCPs (Group I HCPs heat shock cognate protein; pyruvate kinase; actin, cytoplasmic 1; phopsphoglycerate mutase 1; vimentin; clusterin; elongation factor 2; nidogen-1; sulfated glycoprotein 1; glutathione s-transferase P; alpha-enolase; cofilin-1; aldose reductase-related protein; elongation factor I-alpha; Group I/II proteins cathepsin B; matrix metalloproteinase-9; matrix metalloproteinase-19; serine protease HTRA1; Group II proteins sialidase I; endoplasmic reticulum BiP; and Group III proteins phospholipase B-like protein and procollagen-lysine, 2-oxogluarate 5-dioxygenase 1) compared to Capto Q. Furthermore, 5 of the 37 species tracked were more effectively bound to 4HP compared to Capto Adhere (pyruvate kinase, vimentin, clusterin, sulfated glycoprotein 1, and serine protease HTRA1). The remaining species in both cases showed no significant difference in spectral abundance, and, as a result, no problematic HCPs were found to be captured more effectively by Capto Q than 4HP. The 6HP resin was also successful in binding these HCPs compared to Capto Q, showing significantly lower spectral abundance for 22 of the 37 investigated species, comprising Group I HCPs heat shock cognate protein; pyruvate kinase; actin, cytoplasmic 1; phopsphoglycerate mutase 1; vimentin; clusterin; elongation factor 2; nidogen-1; sulfated glycoprotein 1; cofilin-1; aldose reductase-related protein; elongation factor I-alpha; Group I/II proteins lipoprotein lipase; cathepsin B; matrix metalloproteinase-9; matrix metalloproteinase-19; serine protease HTRA1; Group II proteins sialidase I; endoplasmic reticulum BiP; Group I/III protein peroxiredoxin-1; and Group III proteins phospholipase B-like protein and procollagen-lysine, 2-oxogluarate 5-dioxygenase 1.

In comparison to Capto Adhere, 7 of the 37 species were bound more effectively by 6HP, comprising heat shock cognate protein, pyruvate kinase, vimentin, clusterin, phospholipase B-like protein, cofilin-1, and serine protease HTRA1. Only 1 HCP, Group I HCP peptidyl-prolyl cis-trans isomerase, showed statistically higher binding to Capto Adhere. Species more effectively captured by 4HP and 6HP compared to benchmark resins showed good agreement, as expected given similarities in peptide functional groups.

Among the peptide-based resins, 4MP showed the lowest improvement in HCP binding compared to Capto Q and Capto Adhere; nonetheless, improved problematic HCP capture was observed, and was noted to be associated with the lowest mAb product binding as detailed in prior work (Lavoie et al., 2019). 13 of the 37 considered species showed significantly lower spectral abundance (higher binding) compared to Capto Q, including Group I HCPs pyruvate kinase, vimentin, clusterin, elongation factor 2, nidogen-1, sulfated glycoprotein 1, and elongation factor 1-alpha; Group I/II HCPs cathepsin B and serine protease HTRA1; Group II HCPs sialidase 1 and endoplasmic reticulum BiP; and Group III HCPs phospholipase B-like protein and procollagen-lysine, 2-oxogluarate 5-dioxygenase 1. One HCP, Group I/II HCP Cathepsin D, was bound more effectively by Capto Q than 4MP, but overall, significantly improved binding performance was observed. Capto Adhere binding of problematic HCPs outperformed 4MP only for 5 species, namely heat shock cognate protein, cathepsin B, sulfated glycoprotein 1, phospholipase B-like protein, and endoplasmic reticulum BiP; however, the high mAb product binding observed with this resin would reduce the likelihood of its implementation. 4MP outperformed Capto Adhere with a single protein, Group I/II HCP serine protease HTRA1. While 4MP resin returned the lowest HCP binding performance, it should be noted that by both quantitative and qualitative measures, it outperforms quaternary amine ligands (Capto Q), which are currently employed on depth filtration media for clearing HCPs in harvest fluids that feature comparable salt concentration to that considered here (Gilgunn et al., 2019; Singh et al., 2017).

Finally, 6MP behaved similarly to 6HP in improving the clearance of HPC species compared to Capto Q, with the only exceptions of pyruvate kinase and lipoprotein lipase. Compared to Capto Adhere, no statistically significant difference was observed in the binding of the 37 species of problematic HCPs; however, a significantly lower binding of the mAb product was reported, confirming previous findings of enhanced selectivity compared to Capto Adhere (Lavoie et al., 2019).

### Example 3

### Capture of HCP Species by Peptide Ligands Under Dynamic Binding Conditions

In this Example, performance of selected peptide resins (4MP, 6HP, and a mixture of peptides from both resins, 6HP+4MP) were evaluated in dynamic binding conditions to further characterize the ability of these resins to clear HCPs from direct application of mAb production harvest. In Examples 1-3, the lowest pH condition tested, pH 6.0, showed the most selective clearance of HCPs at salt conditions most closely simulating that of the harvest. As a result, clarified cell culture harvest titrated to pH 6.0 was used to test these resins in dynamic binding conditions. 4MP and 6HP were selected due to the diversity in their capture of HCPs from prior work (Examples 1-3). 6HP, while observed to have the highest affinity for mAb product of the peptide resins tested (K_{p,mAb}=0.96 for the pH 6, 150 mM condition), also demonstrated binding of the largest number of unique proteins. 4MP was included as the highest observed HCP selectivity candidate of the resins tested. The resulting impurities profile as determined by size exclusion chromatography indicates that in dynamic binding mode, the 6HP and 4MP ligands are useful in high yield impurities capture. 4MP was shown to bind more selectively to high molecular weight impurities, while 6HP was more effective for binding of low molecular weight impurities. Furthermore, it was shown that mixing these resins to create the 6HP+4MP resin was as effective in clearing both high and low molecular weight impurities as the individual resins.

Materials. For preparation of peptide resins, Toyopearl AF-Amino-650M resin was obtained from Tosoh Corporation (Tokyo, Japan). Fluorenylmethoxycarbonyl- (Fmoc-) protected amino acids Fmoc-Gly-OH, Fmoc-Ser(tBu)-OH, Fmoc-Ile-OH, Fmoc-Ala-OH, Fmoc-Phe-OH, Fmoc-Tyr(tBu)-OH, Fmoc-Asp(OtBu)-OH, Fmoc-His(Trt)-OH, Fmoc-Arg(Pbf)-OH, Fmoc-Lys(Boc)-OH, Fmoc-Asn(Trt)-OH, and Fmoc-Glu(OtBu)-OH, Hexafluorophosphate Azabenzotriazole Tetramethyl Uronium (HATU), diisopropylethylamine (DIPEA), piperidine, and trifluoroacetic acid (TFA) were obtained from ChemImpex International (Wood Dale, Ill., USA). Kaiser test kits, triisopropylsilane (TIPS), and 1,2-ethanedithiol (EDT) were obtained from Millipore Sigma (St. Louis, Mo., USA). N,N′-dimethylformamide (DMF), dichloromethane (DCM), methanol, and N-methyl-2-pyrrolidone (NMP) were obtained from Fisher Chemical (Hampton, N.H., USA).

For dynamic binding studies, CHO-K1 mAb-producing clarified cell culture harvest was generously provided by Fujifilm Diosynth Biotechnologies (Durham, N.C., USA). Sodium phosphate (monobasic), sodium phosphate (dibasic), hydrochloric acid, sodium hydroxide, Bis-Tris, ethanol, and sodium chloride were obtained from Fisher Scientific (Hampton, N.H., USA). Vici Jour PEEK 2.1 mm ID, 30 mm empty chromatography columns and 10 μm polyethylene frits were obtained from VWR International (Radnor, Pa., USA). The Yarra 3 μm SEC-2000 300×7.8 mm size exclusion chromatography column was obtained from Phenomenex Inc. (Torrance, Calif., USA). Repligen CaptivA Protein A chromatography resin was generously provided by LigaTrap Technologies (Raleigh, N.C., USA).

Solid Phase Peptide Synthesis and Side Chain Deprotection. The 611HP peptides RYYYAI-GSG (SEQ ID NO: 2), HSKIYK-GSG (SEQ ID NO: 5), GSRYRY-GSG (SEQ ID NO: 1), IYRIGR-GSG (SEQ ID NO: 4), and AAHIYY-GSG (SEQ ID NO: 3), and the 4MP peptides DKSI-GSG (SEQ ID NO: 15), DRNI-GSG (SEQ ID NO: 16), HYFD-GSG (SEQ ID NO: 17), and YRFD-GSG (SEQ ID NO: 18) were synthesized on Toyopearl AF-Amino-650M (˜0.1 mmol amine/mL resin loading, 0.6 mL settled volume per reaction vial) via conventional Fmoc/tBu chemistry as described in Examples 1-3 using a Biotage Syro II automated parallel synthesizer. Prior to synthesis, Toyopearl resin was swollen in DMF for 20 min at 40° C. All amino acid couplings were performed by incubating the resin with Fmoc-protected amino acid (3 equivalents compared to the amine functional density of the resin), HATU (3 eq.), and DIPEA (6 eq.) at 65° C. for 20 min. Multiple amino acid couplings were repeated at each position to ensure complete conjugation; reaction completion was monitored by Kaiser test. Following amino acid conjugation, Fmoc deprotection was performed using 20% v/v piperidine in DMF at room temperature for 10 minutes, followed copious DMF washing; for the 6HP sequences, a second deprotection step with 40% v/v piperidine in DMF at room temperature for 3 minutes was included for the last two positions. After chain elongation, the peptides were washed with DMF, DCM, and deprotected by acidolysis using a cocktail comprising 95% TFA, 3% TIPS, 2% EDT, and 1% water (10 mL per mL of resin) at room temperature for 2 hours under mild stirring. The resin was drained, and washed sequentially with DCM, DMF, methanol, and stored in 20% v/v aqueous methanol. Aliquots of the peptide-Toyopearl resins were analyzed by Edman degradation to validate the peptide sequences. The 4MP-Toyopearl resin was formulated by mixing equal volumes of DKSI-GSG-Toyopearl (SEQ ID NO: 15), DRNI-GSG-Toyopearl (SEQ ID NO: 16), HYFD-GSG-Toyopearl (SEQ ID NO: 17), and YRFD-GSG-Toyopearl resins (SEQ ID NO: 18); similarly, the 6HP-Toyopearl resin was formulated by mixing equal volumes of RYYYAI-GSG-Toyopearl (SEQ ID NO: 2), HSKIYK-GSG-Toyopearl (SEQ ID NO: 5), GSRYRY-GSG-Toyopearl (SEQ ID NO: 1), IYRIGR-GSG-Toyopearl (SEQ ID NO: 4), and AAHIYY-GSG-Toyopearl (SEQ ID NO: 3); finally the 4MP/6HP-Toyopearl resin was formulated by equal volume mixing of all peptide-Toyopearl resins.

Capture of CHO HCPs in dynamic mode using 4MP-Toyopearl, 6HP-Toyopearl, 4MP/6HP-Toyopearl resins. Dynamic binding experiments were performed using an AKTA Pure 25 L FPLC (GE Healthcare Life Sciences, Chicago, Ill., USA). A volume of 0.1 mL of 6HP-Toyopearl, 4MP-Toyopearl, and 6HP/4MP-Toyopearl resins were wet packed in Vici Jour PEEK 2.1 mm ID, 30 mm column, washed with 20% v/v ethanol (˜10 CVs), deionized water (3 CVs), and finally equilibrated with 10 mM Bis-Tris buffer added with 150 mM sodium chloride at pH 6.0 (10 CVs) at 1.0 mL/min. A volume of 10 mL of clarified CHO-K1 mAb production harvest titrated to pH 6.0 was loaded on the column at the flow rate of either 0.2 mL/min (residence time, RT: 0.5 min), 0.1 mL/min (RT: 1 min), 0.05 mL/min (RT: 2 min), or 0.02 mL/min (RT: 5 min). Flow-through fractions were collected at 1 mL increments, resulting in 17 fractions per injection. Following load, the column was washed with 20 CV of equilibration buffer at the corresponding flow-rate, and a pooled wash fraction was collected until 280 nm absorbance decreased below 50 mAU. All the flow-through runs were performed in triplicate and the resin was discarded after use (no elution or regeneration was performed).

Quantification of mAb in flow-through samples by analytical Protein A chromatography (PrAC). The mAb concentration in the titrated harvest and flow-through fractions was determined by analytical Protein A chromatography using a Waters Alliance 2690 separations module system with a Waters 2487 dual absorbance detector (Waters Corporation, Milford, Mass., USA). Repligen CaptivA Protein A resin packed in a Vici Jour PEEK 2.1 mm ID×30 mm column (0.1 mL) was equilibrated with PBS, pH 7.4. A volume of 10 μL for each sample or standard was injected, and the analytical method proceeded as outlined in Table 4. The effluent was monitored by 280 nm absorbance (A280), and the concentration was determined based on the peak area of the A280 elution peak. Pure mAb at 0.1, 0.5, 1.0, 2.5, and 5.0 mg/mL was utilized to construct the standard curve.

TABLE 4
HPLC Gradient for mAb Quantification by
Analytical Protein A Chromatography

| Time | Flowrate | | |
|---|---|---|---|
| (min) | (mL/min) | % Buffer A | % Buffer B |
| 0.00 | 0.5 | 100% | 0% |
| 2.00 | 0.5 | 100% | 0% |
| 2.01 | 0.5 | 0% | 100% |
| 6.00 | 0.5 | 0% | 100% |
| 6.01 | 0.5 | 100% | 0% |
| 10.00 | 0.5 | 100% | 0% |

To assess the recovery of mAb product, the values of pooled yield as a function of CV were calculated using the equation below.

$$Yield = \frac{\sum {C}_{f , mAb} \times {V}_{f}}{{C}_{L , mAb} \times {V}_{L}}$$

Wherein C_{f,mAb} is the mAb concentration in flow-through fraction f, V_{f} is the volume of flow-through fraction f, C_{L,mAb} is the mAb concentration in the titrated cell culture harvest loaded, and V_{L} is the cumulative feed volume loaded.

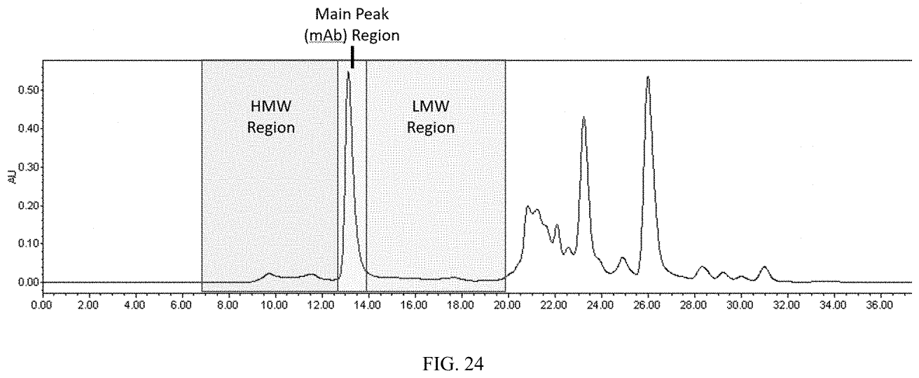

Quantification of low molecular weight (LMW) and high molecular weight (HMW) HCPs in flow-through fractions by size-exclusion chromatography (SEC). The flow-through fractions were then analyzed by analytical SEC using a Yarra 3 μm SEC-2000 300 mm×7.8 mm column operated with a 40-min isocratic method using PBS at pH 7.4 as mobile phase. A volume of 50 μL of sample was injected and the effluent continuously monitored by UV spectrometry at 280 nm absorbance (A280). The values of relative abundance of HWM and LMW HCPs in the flow-through fractions were calculated as % of the main peak. First, the sum total integrated area of all peaks was calculated; the integrated peak area was then separated into three sections based on retention time relative to the main product peak at ˜150 kDa (FIG. 24), determined using a standard molecular weight ladder; the HMW and LMW peak areas were defined as the integrated areas of all peaks at retention times respectively lower and higher than that of the main peak; the peaks relative to ultra-small molecular weight impurities (MW<10 kDa) were removed from the LMW area; finally, the values of “HMW % of main peak” and “LMW % of main peak” were calculated using the equations below.

$$HMW \% of Main Peak = \frac{{A}_{HMW}}{{A}_{M a i n}} \times 100 \%$$

$$LMW \% of Main Peak = \frac{{A}_{LMW}}{{A}_{M a i n}} \times 100 \%$$

Wherein A_{Main}, A_{HMW}, and A_{HMW} are the integrated main area at 150 kDa (corresponding to the mAb), the high molecular weight peak area (MW>150 kDa), and the low molecular weight peak area (10 kDa<MW<150 kDa), respectively. The cumulative HMW % and LMW % of main peak were calculated using the equation below.

$$HMW {\%}_{C u m u l a t i v e , f} = \frac{{\sum }_{i = 1}^{f} {A}_{HMW , i}}{{\sum }_{i = 1}^{f} {A}_{mAb , i}} \times 100 \%$$

$$LMW {\%}_{C u m u l a t i v e , f} = \frac{{\sum }_{i = 1}^{f} {A}_{LMW , i}}{{\sum }_{i = 1}^{f} {A}_{mAb , i}} \times 100$$

Wherein HMW %_{Cumulafive,f} is the cumulative HMW % at fraction f, A_{HMW,i} is the HMW peak area in the i-th fraction, A_{LMW,i} is the LMW peak area in the i-th fraction, and A_{mAb,i} is the main peak area in the i-th fraction. Finally, the cumulative mAb purity was calculated using the equation below.

$$P u r i t {y}_{Cumulative , f} = \frac{{\sum }_{i = 1}^{f} {A}_{mAb , i}}{{\sum }_{i = 1}^{f} {A}_{H M W , i} + {A}_{mAb , i} + {A}_{L M W , i}} \times 100 \%$$

Wherein Purity_{Cumulative,f} is the cumulative % purity at fraction f, A_{LMW,i} is the LMW peak area in the i-th fraction, A_{HMW,i} is the HMW peak area in the i-th fraction, and A_{mAb,i} is the main peak area in the i-th fraction.

Proteomic analysis of the flow-through fractions by liquid chromatography electrospray ionization tandem mass spectrometry (LC-ESI-MS-MS). The feed and flow-through samples were first processed by filter-aided sample preparation (FASP) using a modified trypsin digest method adapted from the work by Wisniewski et al. (Wisniewski et al., 2009). Briefly, 30 μL of flow-through sample were denatured in 5 mM dithiothreitol at 56° C. for 30 min, washed twice with 8 M urea and once with 0.1 M Tris HCl buffer in 3 kDa MWCO Amicon Ultra 0.5 mL spin filters (EMD Millipore, Darmstadt, Germany), and alkylated with 0.05 M iodoacetamide at room temperature for 20 min. The samples were again washed with 8 M urea, 0.1 M tris HCl, 50 mM ammonium bicarbonate, and finally trypsinized overnight at 37° C. using 15 μg/mL sequencing-grade modified trypsin at a trypsin:protein ratio of ˜1:100. Following trypsinization, samples were washed again with 50 mM ammonium bicarbonate, evaporated to dryness by speed-vac, reconstituted in 1 mL aqueous 2% acetonitrile, 0.1% formic acid (mobile phase A), and then further diluted 1:5 in mobile phase A prior to injection. Proteomics analysis with nanoLC-MS/MS was performed at the Molecular Education, Technology, and Research Innovation Center (METRIC) at NC State University. Samples were loaded as 2 μL injections and proteins were separated using a 60-min linear gradient at 300 nL/min of mobile phase A and mobile phase B (0.1% formic acid in acetonitrile) from 0-40% mobile phase B. The operational parameters of the Orbitrap were (i) positive ion mode, (ii) acquisition—full scan (m/z 400-1400) with 120,000 resolving power in MS mode, (iii) MS/MS acquisition using top 20 data dependent acquisition implementing higher-energy collisional dissociation (HCD) using normalized collision energy (NCE) setting of 27%; dynamic exclusion was adopted to minimize re-interrogation of previously sampled precursor ions. The resulting nanoLC-MS/MS data were processed using Proteome Discoverer 2.2 (Thermo Fisher, San Jose, Calif.) by performing a search with a 5 ppm precursor mass tolerance and 0.02 Da fragment tolerance against a Cricetulus griseus (Chinese hamster) CHOgenome/EMBL database. The database search settings were specific for trypsin digestion and included modifications such as dynamic Met oxidation and static Cys carbamidomethylation. Identifications were filtered to a strict protein false discovery rate (FDR) of 1% and relaxed FDR of 5% using the Percolator node in Proteome Discoverer.

Relative Quantification of Individual HCPs and Bound Protein Analysis. A relative quantification of HCPs in the flow-through samples was obtained from the MS-derived spectral count (SpC) of every HCP (Cooper et al., 2010). Percent removal of individual proteins in the collected supernatants samples (combination of the unbound fraction from the static binding and the following wash) was calculated as shown in the equation below.

$${SAF}_{i , j} = \frac{S p {C}_{i , j} \times D {F}_{j}}{{L}_{i}}$$

Wherein SAF_{i,j} is the spectral abundance factor for protein i in sample j (kDa⁻¹), SpCᵢ is the spectral count of protein i in sample j, DFⱼ is the Dilution factor for sample j, and Lᵢ is the length of protein i (kDa). The relative abundance of every HCP in the feed sample was calculated based on normalized spectral abundance factor (NSAF) (Neilson et al., 2013) for each identified protein. A comparison of the relative quantities of individual HCPs in the flow-through vs. feed samples was finally conducted by Analysis of Variance (ANOVA) of the spectral counts for every protein using JMP Pro 14.

For the analysis of bound HCPs, the protein spectral counts were used to compare the flow-through fractions obtained using 4MP-Toyopearl, 6HP-Toyopearl, 4MP/6HP-Toyopearl resins. “Bound HCPs” are herein defined as the proteins that (i) were identified in the majority of feed samples (i.e., had a sum of spectral count greater than 4 across all replicates, N=3) and (ii) were either not found in the supernatant samples or showed significantly lower spectral count (p<0.05 by ANOVA) compared to the feed sample. Venn diagrams of bound proteins across peptide-based and benchmark resins were constructed using the Venn diagram add-in for JMP Pro 14 (FIGS. 28-31). The non-normal distributions for isoelectric points of depleted proteins were compared by Kruskal-Wallis H test with a 90% confidence interval using JMP Pro 14.

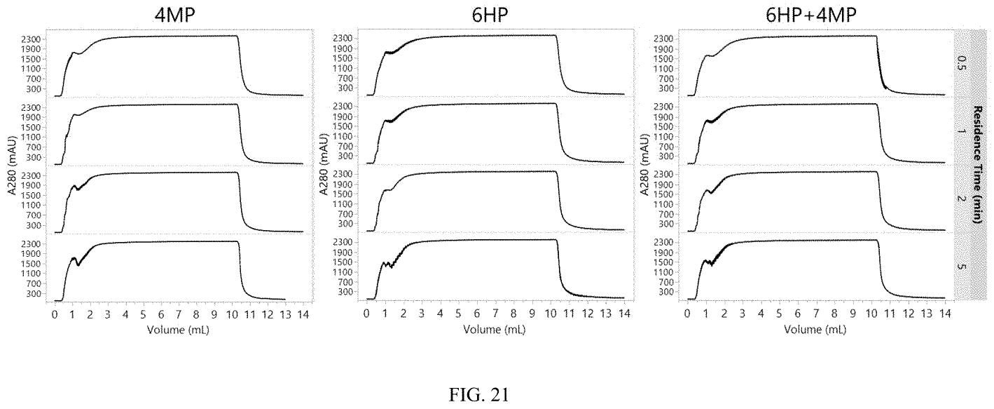

HCP-Selective Peptide Resins in Dynamic BindingMode. The HCP-targeting peptides 61HP (GSRYRYGSG (SEQ ID NO: 19), HSKIYKGSG (SEQ ID NO: 23), IYRIGRGSG (SEQ ID NO: 22), AAHIYYGSG (SEQ ID NO: 21), and RYYYAIGSG (SEQ ID NO: 20)) and 4MP (YRFDGSG (SEQ ID NO: 36), DKSIGSG (SEQ ID NO: 33), DRNIGSG (SEQ ID NO: 34), and HYFDGSG (SEQ ID NO: 35)) were individually synthesized on Toyopearl AF-Amino-650M resin as described in Example 2-3. The resulting resins were mixed in equal volumes to generate the adsorbents (i) 6HP-Toyopearl resin, comprising the five 6HP peptides, (ii) 4MP-Toyopearl resin, comprising the four 4MP peptides, and (iii) 6HP+4MP-Toyopearl resins, comprising all nine peptides. The three adsorbents were packed in 0.1 mL columns, and equilibrated with 10 mM Bis-Tris added with 150 mM sodium chloride at pH 6.0. A volume of 10 mL of clarified CHO-K1 IgG1 production harvest (˜1.7 g total protein/L and ˜1.4 mg/mL mAb) was loaded onto the columns at different residence times (0.5, 1, 2, and 5 min), resulting in a total protein load of ˜170 mg of protein per mL resin. The effluent was continuously monitored by UV spectroscopy at 280 nm and collected at incremental fractions of 1 mL. The resulting chromatograms (FIG. 21) do not show any conspicuous difference; given the low abundance of the HCP species relative to the mAb product (HCP:IgG˜1:5), the A280 signal of the effluent is mostly determined by the mAb.

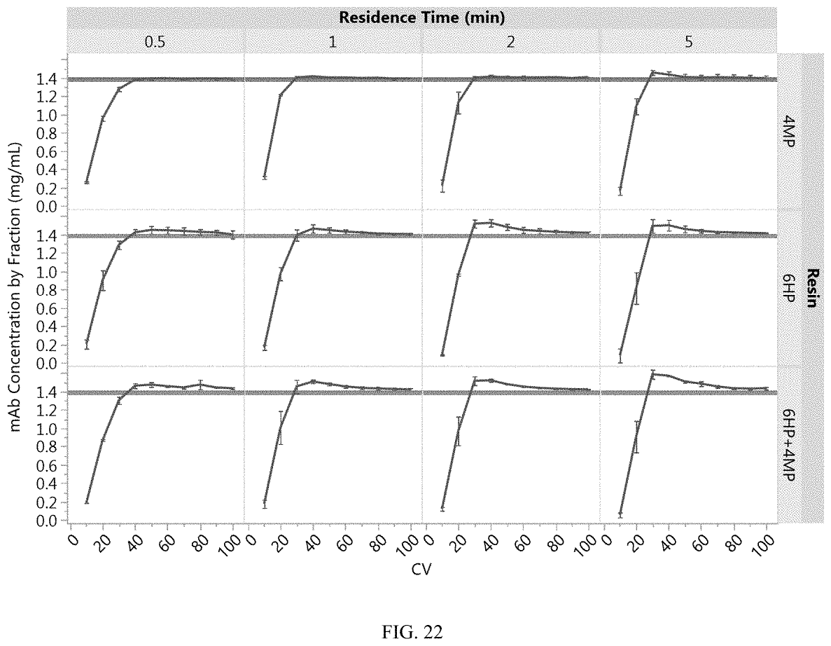

Binding of mAb and mAb Product Yield. Binding of the mAb product to the peptide resins was monitored for this work to evaluate potential for product loss. The mAb concentration in each fraction and in the feed, as determined by analytical Protein A chromatography, is reported in FIG. 22. Upon inspection of the mAb concentration for each resin, higher concentrations of the mAb relative to the feed concentration were observed, corresponding with stabilization of the A280 dynamic binding chromatogram shown in FIG. 21. This effect is particularly pronounced for the 6HP and 6HP+4MP resins, with increasing maximum concentrations for each resin correlating with increasing residence time. In Examples 1-3, higher mAb product binding in static binding mode for the 6HP resins was observed compared to the 4MP resins, and it was additionally noted that the a larger fraction of the fed HCPs bound to the peptide resins as compared to the mAb product. Given the stronger binding of mAb by the 6HP resin, the observed increase in concentration is potentially a result of partitioning. This is supported by previous work in static binding mode (Examples 1-3), where the Kₚ of the mAb product for 6HP was higher compared to 4MP⁶⁹ (K_{p,mAb}=0.96 for 6HP compared to 0.75 for 4MP at pH 6, 150 mM sodium chloride). 6HP's higher observed affinity for mAb product likely corresponds to a larger fraction of mAb bound at low loading in dynamic binding mode. This increased binding, coupled with HCP Kₚ an order of magnitude larger than the mAb product (K_{p,HCP}=7.3 and 6.1 for 4MP and 6HP, respectively at pH 6, 150 mM sodium chloride in static binding conditions) may explain this trend. Upon loading of the harvest, highly abundant mAb molecules weakly bind and saturate the ligand such that when further harvest is introduced, higher affinity HCPs displace the weakly bound mAb, resulting in the observed increased mAb concentration. This shows that these ligands are optimally operated in WPC for direct application of titrated harvest.

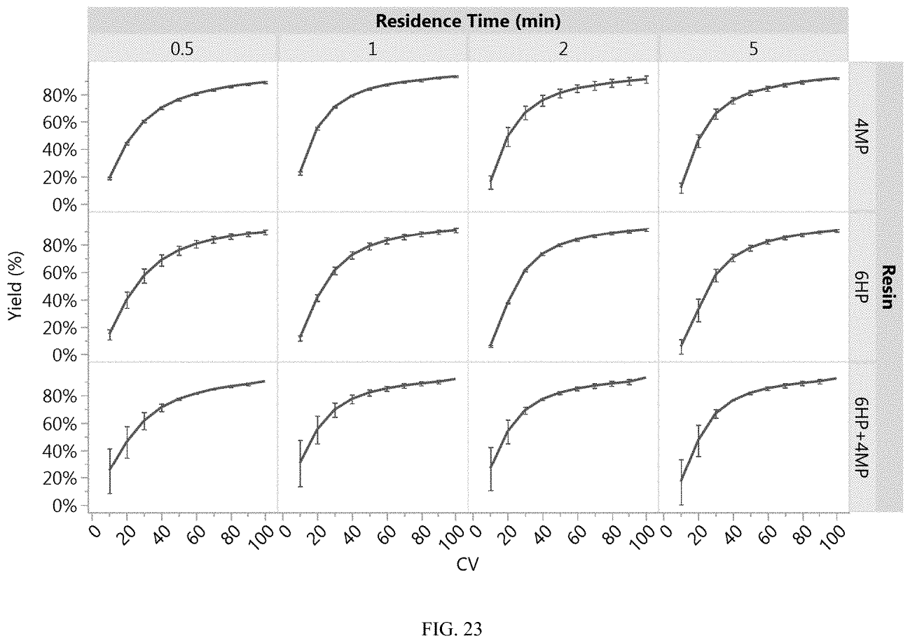

To assess mAb product recovery, the pooled yield as a function of load was calculated as shown in the equation the below for comparison by residence time and resin as shown in FIG. 23. Note that the calculated pooled yield does not incorporate any washing of the column.

$$Yield = \frac{\sum {C}_{f , m A b} \times {V}_{f}}{{C}_{L , m A b} \times {V}_{L}}$$

Calculated pooled yield, where C_{f,mAb} is the mAb concentration in flow through fraction f, V_{f} is the volume of flow through fraction f, C_{L,mAb} is the mAb concentration in the titrated cell culture harvest loaded, and V_{L} is the cumulative feed volume loaded.

For the conditions tested, all resins exceeded 80% mAb product yield by 120 mg total protein/mL load, the approximate load that the mAb fraction concentration sinks to the feed concentration. This observation, coupled with improved yield observed with increasing residence time, further supporting weak partitioning of the loaded proteins. For 1, 2, and 5 min residence times, pooled yield exceeded 90% by the highest load tested, 200 mg/mL for all resins.

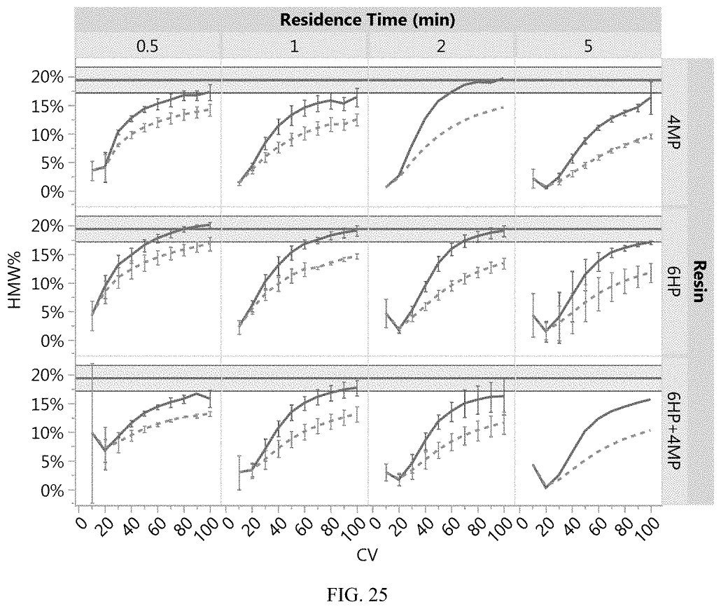

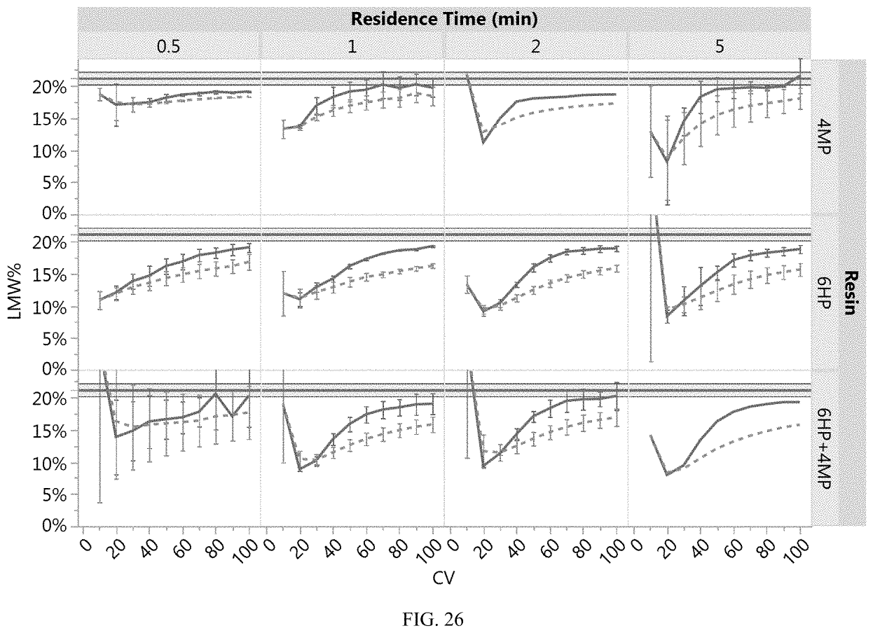

Clearance of High and Low Molecular Weight Impurities by HCP-Selective Peptide Resins. The titrated feed and flow-through fractions were also analyzed by size exclusion chromatography (SEC) to derive qualitative correlations between the clearance of high molecular weight (MW>150 kDa) and low molecular weight (10 kDa<MW<150 kDa) HCPs and the ligand type, protein load, and residence time. The resulting absorbance chromatogram as monitored at 280 nm was then interpreted by determining the total area under all signal observed in the relevant range for proteins, followed by separation of the integration area into three distinct regions: (i) high molecular weight (HMW), (ii) main peak (IgG), and (iii) low molecular weight (LMW) as summarized in FIG. 24. With this, we sought to obtain a preliminary understanding of the conditions that optimize clearance of high and low molecular weight HCP impurities. To this end, the chromatograms were divided in three regions, namely (i) high molecular weight (HMW, SEC residence time<12.8 min), (ii) main peak (mAb product and potential HCPs with similar hydrodynamic radius), and (iii) low molecular weight (LMW, SEC residence time between 13.6-20 min). The integrated chromatogram areas corresponding to these regions were utilized to calculate fractional and cumulative ratios of HMW:main peak area, or “HMW %”, and LMW:main peak area, or “LMW %”, using the equations outlined above and compared among different resins, load volumes, and residence times. FIG. 25 and FIG. 26 respectively report the resulting values of fractional (solid curves) and cumulative (dashed curves) HMW % and LMW % vs. CV loaded obtained at different residence times using 4MP-Toyopearl, 6HP-Toyopearl, and 4MP/6HP-Toyopearl resins. The plots of cumulative HMW % and LMW % of main peak represent the simulated HMW % and LMW % that would be obtained by pooling the flow-through fractions.

The relatively slow increase in flow-through HMW % as the loading of harvest on the resin progresses, consistently observed across all residence times, indicates that the peptide-based resins possess high binding strength and capacity for HMW HCPs. In particular, when operated at 5 min residence time, 4MP-Toyopearl resin provided highly effective capture of HMW HCPs, reaching a cumulative HMW % of 5.8% at the cut-off value of load (60 CV, corresponding to a loading of ˜102 mg protein per mL resin), at which a 84% mAb yield is obtained; this translates in the capture of 70% of fed HMW HCPs. At maximum load (10 CV or 170 mg/mL loading), at which a mAb yield of 91% is obtained, a 9.6% HMW % was observed, which corresponds to a removal of 51% of fed HMW HCPs. In contrast, 6HP-Toyopearl resin operated at 5 min residence time afforded a HMW % of only 8.0% at the 60 CV cut-off load, equating to a 59% removal of HMW HCPs, and 11.8% at maximum load, equating to a HMW HCP removal of 11.8%.

Most notably, the combined 4MP/6HP-Toyopearl resin afforded a remarkable 2-to-4-fold reduction in HMW species during the early stages of loading (10-30 CV), while at the cut-off load a HMW % of 6.5% was obtained, corresponding to the removal of 65% of HMW HCPs in the feed, and 10.9% at the maximum load, corresponding to a 44% removal. This indicates that 4MP- and 6HP-Toyopearl resins target different HMW HCPs, and must be operated together in order to grant mAb purification in flow-through mode. At 1 min residence time, which represents a technologically relevant operating condition, the HMW % at the cut-off load was ˜10% for 4MP-Toyopearl and 6HP/4MP-Toyopearl resins, corresponding to the capture of 49% of fed HMW HCPs, and 12.4% for 6HP-Toyopearl, corresponding to a 36.4% capture; at maximum load, instead, the HMW % increased to 12.5% and 13.2%, corresponding to the removal of 36% and 32% of fed HMW HCPs, for 4MP-Toyopearl and 6HP/4MP-Toyopearl resins, compared to 14.7% (25% removal) for 6HP alone. Collectively, these results demonstrate the cooperation in HCP binding by 4MP and 6HP peptides. This confirms prior studies on HCP capture by the peptide ligands (Examples 1-3), which showed that the populations of HCPs bound by the two groups of peptides overlap to some extent, but also comprise a number of species that are uniquely captured by 4MP and 6HP.

The corresponding analysis of the LMW HCPs showed an opposite trend compared to that of HMW HCPs, wherein 6HP and combined 6HP/4MP ligands showed higher binding strength and capacity compared to 4MP ligands. 4MP-Toyopearl resin, in fact, afforded low clearance of LMW HCPS, with <25% of fed proteins captured, at loads above 60 CV, where the values of mAb yield would be industrially viable (>80%), across all residence times. On the other hand, 6HP-Toyopearl and 6HP/4MP-Toyopearl resins, when operated at 5 min residence time, captured ˜37% of fed LMW HCPs at the cut-off value of load (60 CV, corresponding to mAb yield>80%), and 25% at the maximum load (100 CV, mAb yield of >90%); when operated at 5 min residence time, instead, they respectively afforded 29% and 34% captures at the cut-off value of load, and ˜18% capture at maximum load. Improved clearance of LMW species was consistently observed when operating at higher residence time, particularly for the 6HP-Toyopearl and 6HP/4MP-Toyopearl resins. As mentioned above (Examples 1-3), prior studies in static binding mode indicated substantial differences in the binding of individual HCPs by the different resins, which corroborates the differences observed in both % HMW and % LMW to main peak trends between the two ligand sets. Proteomic analysis of the cell culture harvest has shown that species with MW<100 kDa account for the majority of the HCP population, suggesting that the clearance of total HCPs can rely on resins with high binding strength and capacity for LMW species. Under this premise, the results presented above are consistent with prior data produced in static binding mode In Examples 2-3, where a statistically significant clearance of a larger number of unique HCPs was observed for 6HP resin as compared to 4MP.

To easily compare the purification performance of the peptide-based resins, the values of mAb purity in the flow-through fractions calculated using the following equation

$${Purity}_{Cucumulative , f} = \frac{{\sum }_{i = 1}^{f} {A}_{mAb , i}}{{\sum }_{i = 1}^{f} {A}_{HMW , i} + {A}_{m A b , i} + {A}_{LMW , i}} \times 100 \%$$

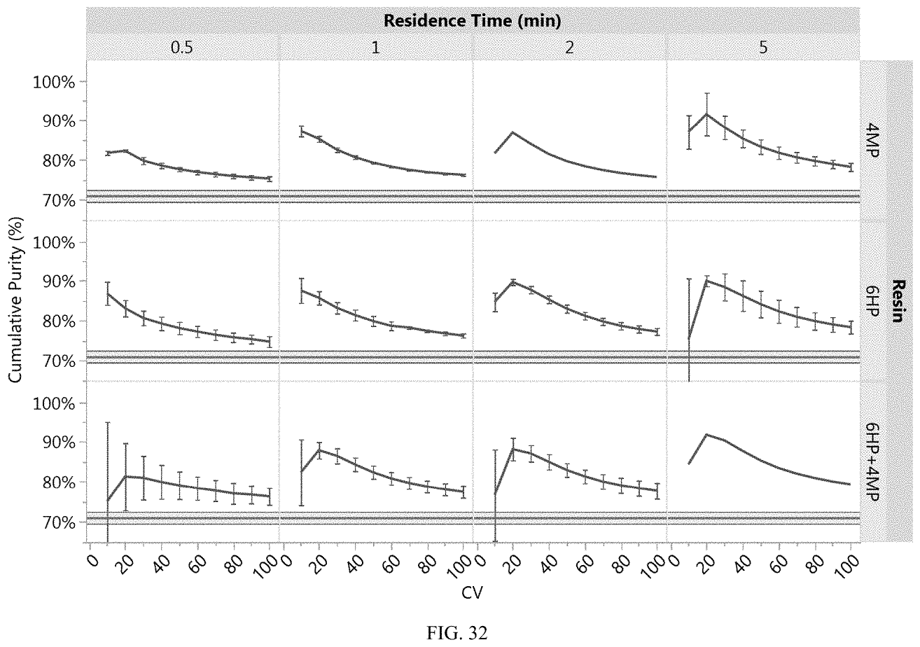

And are demonstrated in FIG. 32 as functions of loading (CV) and residence time. The maximum mAb purity (91.8%) was obtained using 6HP/4MP-Toyopearl resins operated at 5 min residence time and loaded with 20 CVs of titrated harvest; high purity, however, came at a cost of extremely low product yield (47.1%). Nonetheless, it is noted that the mAb purity in all flow-through fractions was higher than the control range for all resins tested (excluding the fraction corresponding to 10 CVs loading, likely due to the poor sensitivity in the SEC assay), and increased consistently by increasing residence time. When operated at 5 min residence time, all peptide-based resins afforded mAb purity of 82-84% at the 60 CV cut-off load, corresponding to 38-44% decrease in HCP impurities compared to the feed. At 1 and 2 min residence times, which are more technologically relevant, cumulative purity decreased only slightly to 78-81%, and clear binding of harvest impurities was clearly observed.

The values of cumulative purity and yield as functions of loading (CV), residence times, and peptide-based adsorbent were collated. When operated at 1-2 min residence time, a column packed with 6HP/4MP-Toyopearl resin loaded with 50 CVs of titrated cell culture harvest provides a product recovery of ˜80% and a purity of 85%. Given that the initial mAb purity is 72%, flowing the clarified harvest through the 6HP/4MP-Toyopearl adsorbent provides a significant reduction of the overall HCP load, which can return significant benefits in terms of Protein A performance and lifetime

Proteomic analysis of flow-through fractions. The values of global HCP removal represent only one aspect of the purification activity enabled by 4MP and 6HP ligands. Prior studies in static binding mode, in fact, have demonstrated the ability of these ligands to remove “problematic” HCPs, namely species that co-elute with the mAb product from the Protein A column (Group I), species that cause mAb degradation (Group II), and species that are reported as highly immunogenic (Group III). Targeting and removing these species as early as possible in the purification train holds great promise towards increasing product safety and enhancing the performance of downstream bioprocessing.

To assess the binding of individual HCPs by the peptide-based resin, the relative abundance of each species was measured by LC/MS/MS-based proteomic analysis and compared to that of the feed stream by analysis of variance (ANOVA). The method of qualitative bound protein analysis method utilized in this study has been described in detail in Examples 1-3. Briefly, a HCP is considered bound if (i) it is identified in the feed but is not identified in the flow-through, or (ii) the measured spectral abundance factor (a measure of relative concentration calculated using the equation below,

$${SAF}_{i , j} = \frac{S p {C}_{i , j} \times D {F}_{j}}{{L}_{i}}$$

in the flow-through sample is statistically lower (α≤; 0.05 by ANOVA) as compared to the spectral abundance in the feed. Owing to their higher performance compared to 4MP and 6HP ligands alone, the 6HP/4MP combination only was evaluated. Further, only the residence times of 1 min and 2 min were considered, given their technological relevance compared to 5 min and better HCP capture compared to 0.5 min. Finally, the load condition was limited to the values of 40 CV, 50 CV, 60 CV, and 70 CV, which represents the load range near the minimum acceptable thresholds for yield and purity (>80% yield, >80% purity). Under these load conditions, the fractions were pooled prior to analysis such that the 40 CV load condition represents the total HCP concentration for the pooled flow-through of the 10, 20, 30, and 40 CV fractions, the 50 CV condition was the pooled flow-through of the 10, 20, 30, 40, and 50 CV fractions, etc. to evaluate the cumulative, rather than fractional, HCP capture performance.

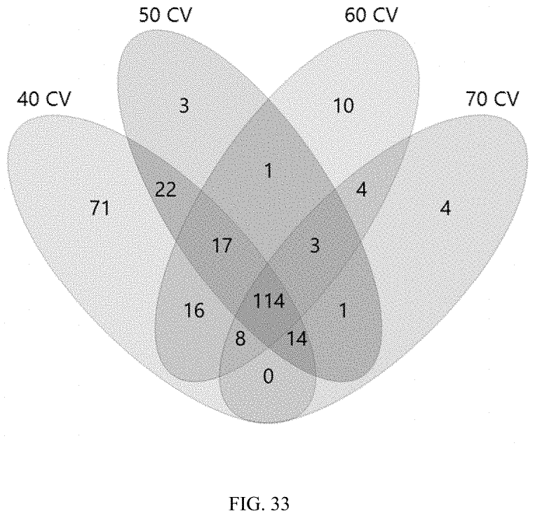

FIG. 33 compares the total number of HCPs that, out of the 661 species identified in the feed stream, are captured by 6HP/4MP-Toyopearl resin at the various load values (CV) at 1 min RT. As anticipated, the highest number of bound proteins was observed at the lowest load condition tested (40 CV) at 292 total proteins bound, representing ˜44% of the total species identified in the feed stream. At the 60 CV cut-off load, 169 HCP species (˜26%) were shown to be captured by the 6HP/4MP ligands. A total of 114 HCP species (˜17% of the species identified in the feed) were observed to bind across all loading conditions, indicating strong binding to the peptide ligands. Most notably, a conspicuous number of known “problematic” HCP species, identified in Examples 2-3, were included in this set of 114 highly-bound species, as summarized in Table 5.

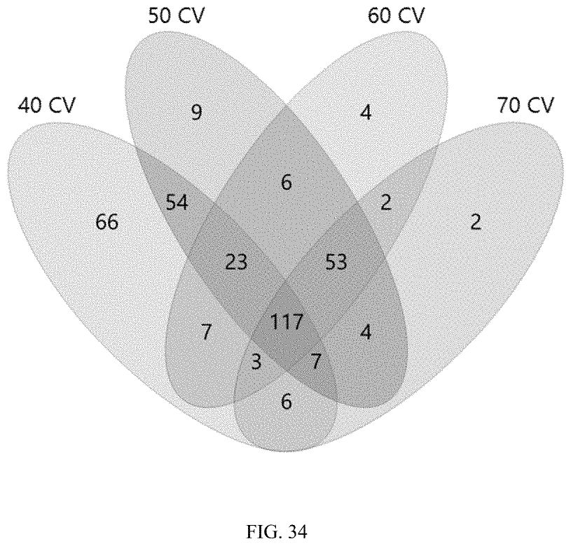

The analysis of bound HCPs was repeated on the fractions generated at 2 min RT, as shown in FIG. 34. A slight decrease in the number of proteins bound at the 40 CV load was observed, with 283 bound species at 2 min RT compared to the 292 bound species at the RT of 1 min, which can be ascribed to a small variability in the results. On the other hand, a notable increase was observed in the number of bound species at the 60 CV cut-off load, with 215 species (33%) bound at the RT of 2 min compared to 169 species bound at the RT of 1 min. This increase in bound HCPs aligns with the increased mAb purity at higher residence time indicated by both SEC and ELISA analysis. At the RT of 2 min, 117 HCP species were observed to bind at all 4 loading conditions, similarly to the 114 species bound at the RT of 1 min.

The ability of the 6HP/4MP peptides to capture a significant fraction of the HCPs present in the feed stream is, from a thermodynamics standpoint, quite remarkable. These proteins are individually present at a concentration ranging between 0.1 and 1 μg/mL, and therefore a molarity likely comprised between 1 and 10 nM. At the same time, the antibody is present at a concentration of ˜1.4 mg/mL, corresponding to a ˜10 μM concentration. The ability of the peptides to capture HCPs selectively without adjusting the protein concentration or the salt composition, concentration, and pH in the feed is therefore remarkable.

TABLE 5
Problematic HCPs bound by 6HP/4MP-Toyopearl
resin operated at 1 or 2 min RT

| Problematic HCP | HCP Species Depleted | HCP Species Depleted |
|---|---|---|
| Group | at RT of 1 min | at RT of 2 min |
| Group I | 60S acidic ribosomal | 60S acidic ribosomal |
| (co-eluting | protein P2 isoform X1 | protein P1 isoform X1 |
| with mAb from | Biglycan | 60S acidic ribosomal |
| Protein A resin) | Cathepsin B | protein P2 isoform X2 |
| | Cathepsin D | Biglycan |
| | Clusterin | Cathepsin B |
| | Heat shock protein | Cathepsin D |
| | HSP 90 | Clusterin |
| | Nidogen-1 isoform X3 | Heat shock protein |
| | Peptidyl-prolyl cis- | HSP 90 |
| | trans isomerase B | Histone H2B |
| | Protein disulfide | Nidogen-1 isoform X3 |
| | isomerase A6 | Peptidyl-prolyl cis- |
| | Serine protease HTRA1 | trans isomerase B |
| | isoform X2 | Protein disulfide- |
| | SPARC isoform X3 | isomerase A6 |
| | Thrombospondin-1 | Serine protease HTRA1 |
| | isoform X1 | isoform X2 |
| | Vimentin | Thrombospondin-1 |
| | | isoform X1 |
| | | Vimentin |
| Group II | Cathepsin B | Cathepsin B |
| (associated | Cathepsin D | Cathepsin D |
| to mAb | Endoplasmic reticulum | Endoplasmic reticulum |
| degradation) | chaperone BiP Precursor | chaperone BiP precursor |
| | Heat shock protein | Heat shock protein |
| | HSP 90 | HSP 90 |
| | Legumain | Legumain |
| | Protein disulfide | Protein disulfide- |
| | isomerase A6 | isomerase A6 |
| | Serine protease HTRA1 | Serine protease HTRA1 |
| | isoform X2 | isoform X2 |
| Group III | Putative phospholipase | Putative phospholipase |
| (highly | B-like 2 | B-like 2 |
| immunogenic) | | |

“Problematic” HCP species captured at all the four loading conditions are summarized in Table 5. The proteomics analysis indicated that 23 HCPs known as “problematic”, due to their ability to either escape Protein A purification, or degrade the mAb by direct proteolytic activity or by degrading stabilizers during storage, or documented high immunogenicity, were effectively captured by the 4MP/6HP-Toyopearl resin, across all values of loading (CV) and residence time. Of particular notice is the capture of Cathepsin B and D, which are implicated in mAb degradation via heavy chain C-terminal fragmentation resulting in the formation of mAb aggregates serine protease HTRA1 and protein disulphide-isomerase A6, both degradative HCPs that have been found in Protein A eluates, putative phospholipase B-like 2, a strong immunogen, and Legumain, a strong protease that forms acidic charge variants by deamidating asparagine residues on mAbs.

The results in this Example demonstrate that the peptide-based resins of the invention, enable antibody purification in flow-through mode by combining selective capture of high and low molecular weight HCP impurities and high product yield. When utilized individually, 6HP and 4MP ligands feature preferential capture of HCP species in the LMW and HMW regions, respectively. When combined, the ensemble of peptide ligands affords a significant reduction in the HCP level of the cell culture harvest, while providing good product yield. In particular, at the 60 CV cut-off load (˜102 mg/mL), a ˜36% reduction in LMW % and a ˜50% reduction in HMW %, combined with ˜85% mAb yield, were obtained when operating at residence times of 1 min.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the invention, may be made without departing from the spirit and scope thereof.

1. A composition for use in a method of removing one or more host cell proteins from a mixture comprising the one or more host cell proteins and one or more target biomolecules, wherein the composition comprises one or more peptides each independently comprising a sequence selected from the group consisting of GSRYRY (SEQ ID NO: 1), RYYYAI (SEQ ID NO: 2), AAHIYY (SEQ ID NO: 3), IYRIGR (SEQ ID NO: 4), HSKIYK (SEQ ID NO: 5), ADRYGH (SEQ ID NO: 6), DRIYYY (SEQ ID NO: 7), DKQRII (SEQ ID NO: 8), RYYDYG (SEQ ID NO: 9), YRIDRY (SEQ ID NO: 10), HYAI (SEQ ID NO: 11), FRYY (SEQ ID NO: 12), HRRY (SEQ ID NO: 13), RYFF (SEQ ID NO: 14), DKSI (SEQ ID NO: 15), DRNI (SEQ ID NO: 16), HYFD (SEQ ID NO: 17), and YRFD (SEQ ID NO: 18); and
wherein each peptide in the composition has a greater binding affinity for the one or more host cell proteins than for the one or more target biomolecules.

2. The composition of claim 1, wherein the one or more target biomolecules is a protein, an oligonucleotide, a polynucleotide, a virus or a viral capsid, a cell or a cell organelle, or a small molecule.

3. The composition of claim 2, wherein the protein is an antibody, an antibody fragment, an antibody-drug conjugate, a drug-antibody fragment conjugate, a Fc-fusion protein, a hormone, an anticoagulant, a blood coagulation factor, a growth factor, a morphogenic protein, a therapeutic enzyme, an engineered protein scaffold, an interferon, an interleukin, or a cytokine.

4. The composition of claim 1, wherein the one or more host cell proteins is independently selected from the proteome of the host cell expressing the one or more target biomolecules.

5. The composition of claim 4, wherein the one or more host cell proteins is independently selected from the group comprising acidic ribosomal proteins, biglycan, cathepsins, clusterin, heat shock proteins, nidogen-1, peptidyl-prolyl cis-trans isomerase B, protein disulfide isomerase, SPARC, thrombospondin-1, vimentin, histones, endoplasmic reticulum chaperone BiP, legumain, serine protease HTRA1, and putative phospholipase B-like protein.

6. The composition of claim 1, wherein the one or more of the peptides further comprises a linker on the C-terminus of the peptide.

7. The composition of claim 1, wherein the linker comprises a Glyn or a [Gly-Ser-Gly]m, wherein 6≥n≥1 and 3≥m≥1.

8. The composition of claim 1, wherein each peptide independently comprises a sequence selected from the group consisting of GSRYRY (SEQ ID NO: 1), RYYYAI (SEQ ID NO: 2), AAHIYY (SEQ ID NO: 3), IYRIGR (SEQ ID NO: 4), and HSKIYK (SEQ ID NO: 5).

9. The composition of claim 1, wherein each peptide independently comprises a sequence selected from the group consisting of ADRYGH (SEQ ID NO: 6), DRIYYY (SEQ ID NO: 7), DKQRII (SEQ ID NO: 8), RYYDYG (SEQ ID NO: 9), and YRIDRY (SEQ ID NO: 10).

10. The composition of claim 1, wherein each peptide independently comprises a sequence selected from the group consisting of HYAI (SEQ ID NO: 11), FRYY (SEQ ID NO: 12), HRRY (SEQ ID NO: 13), and RYFF (SEQ ID NO: 14).

11. The composition of claim 1, wherein each peptide independently comprises a sequence selected from the group consisting of DKSI (SEQ ID NO: 15), DRNI (SEQ ID NO: 16), HYFD (SEQ ID NO: 17), and YRFD (SEQ ID NO: 18).

12. The composition of claim 1, wherein each peptide independently comprises a sequence selected from the group consisting of GSRYRY (SEQ ID NO: 11), RYYYAI (SEQ ID NO: 2), AAHIYY (SEQ ID NO: 3), IYRIGR (SEQ ID NO: 4), HSKIYK (SEQ ID NO: 5), DKSI (SEQ ID NO: 15), DRNI (SEQ ID NO: 16), HYFD (SEQ ID NO: 17), and YRFD (SEQ ID NO: 18).

13. An adsorbent comprising the composition of claim 1 conjugated to a support.

14. The adsorbent of claim 13, wherein all of the peptides in the composition are conjugated to a single support.

15. The adsorbent of claim 14, wherein the adsorbent comprises a plurality of supports and wherein one or more peptide(s) is conjugated to a single support.

16. The adsorbent of claim 16, wherein the one or more peptide(s) conjugated to a single support are all the same peptide or are different peptides.

17. The adsorbent of claim 13, wherein the support comprises a non-porous or porous particle, a non-porous or porous membrane, a plastic surface, or a fiber.

18. The adsorbent of claim 17 wherein the support comprises polymethacrylate, polyethersulfone cellulose, agarose, chitosan, iron oxide, silica, titania, or zirconia.

19. A method for removing one or more host cell proteins from a mixture comprising the one or more host cell proteins and one or more target biomolecules, the method comprising
a. contacting the mixture with the composition of claim 1.

20. The method of claim 19 wherein the method further comprises,
b. washing the composition or adsorbent to remove one or more unbound target biomolecules into a supernatant or mobile phase; and
c. collecting the supernatant containing the one or more unbound target biomolecules.

21. The method of claim 19, wherein the contacting step comprises a high ionic strength binding buffer or low ionic strength binding buffer.

22. The method of claim 21 wherein the binding buffer at low ionic strength comprises 1-50 mM NaCl.

23. The method of claim 21 wherein the binding buffer at high ionic strength comprises 100-500 mM NaCl.

24. The method of claim 19, wherein the contacting step comprise a low pH buffer of between pH 5-6.7.

25. The method of claim 19 wherein the contacting step comprise a neutral pH buffer of between pH 6.8-7.4.

26. The method of claim 19 wherein the contacting step comprise a high pH buffer of between pH 7.5-9.

27. The method of claim 19 wherein the contacting step comprise a neutral pH and low ionic strength binding buffer, wherein the buffer comprises 20 mM NaCl and has a pH of 7.

28. The method of claim 19 wherein the contacting step comprise a low pH and high ionic strength binding buffer, wherein the buffer comprises 150 mM NaCl and has a pH of 6.

29. The method of claim 19 wherein each peptide independently comprises a sequence selected from the group consisting of GSRYRYGSG (SEQ ID NO: 19), RYYYAIGSG (SEQ ID NO: 20), AAHIYYGSG (SEQ ID NO: 21), IYRIGRGSG (SEQ ID NO: 22), HSKIYKGSG (SEQ ID NO: 23), DKSIGSG (SEQ ID NO: 33), DRNIGSG (SEQ ID NO: 34), HYFDGSG (SEQ ID NO: 35), and YRFDGSG (SEQ ID NO: 36).

30. The method of claim 19, wherein the method is performed under static binding conditions.

31. The method of claim 19, wherein the method is performed under dynamic binding conditions.


SEQUENCE LISTING (60 sequences)

SEQ ID NO: 1 - protein, 6 aa, Artificial Sequence
  Synthetic
GSRYRY

SEQ ID NO: 2 - protein, 6 aa, Artificial Sequence
  Synthetic
RYYYAI

SEQ ID NO: 3 - protein, 6 aa, Artificial Sequence
  Synthetic
AAHIYY

SEQ ID NO: 4 - protein, 6 aa, Artificial Sequence
  Synthetic
IYRIGR

SEQ ID NO: 5 - protein, 6 aa, Artificial Sequence
  Synthetic
HSKIYK

SEQ ID NO: 6 - protein, 6 aa, Artificial Sequence
  Synthetic
ADRYGH

SEQ ID NO: 7 - protein, 6 aa, Artificial Sequence
  Synthetic
DRIYYY

SEQ ID NO: 8 - protein, 6 aa, Artificial Sequence
  Synthetic
DKQRII

SEQ ID NO: 9 - protein, 6 aa, Artificial Sequence
  Synthetic
RYYDYG

SEQ ID NO: 10 - protein, 6 aa, Artificial Sequence
  Synthetic
YRIDRY

SEQ ID NO: 11 - protein, 4 aa, Artificial Sequence
  Synthetic
HYAI

SEQ ID NO: 12 - protein, 4 aa, Artificial Sequence
  Synthetic
FRYY

SEQ ID NO: 13 - protein, 4 aa, Artificial Sequence
  Synthetic
HRRY

SEQ ID NO: 14 - protein, 4 aa, Artificial Sequence
  Synthetic
RYFF

SEQ ID NO: 15 - protein, 4 aa, Artificial Sequence
  Synthetic
DKSI

SEQ ID NO: 16 - protein, 4 aa, Artificial Sequence
  Synthetic
DRNI

SEQ ID NO: 17 - protein, 4 aa, Artificial Sequence
  Synthetic
HYFD

SEQ ID NO: 18 - protein, 4 aa, Artificial Sequence
  Synthetic
YRFD

SEQ ID NO: 19 - protein, 9 aa, Artificial Sequence
  Synthetic
GSRYRYGSG

SEQ ID NO: 20 - protein, 9 aa, Artificial Sequence
  Synthetic
RYYYAIGSG

SEQ ID NO: 21 - protein, 9 aa, Artificial Sequence
  Synthetic
AAHIYYGSG

SEQ ID NO: 22 - protein, 9 aa, Artificial Sequence
  Synthetic
IYRIGRGSG

SEQ ID NO: 23 - protein, 9 aa, Artificial Sequence
  Synthetic
HSKIYKGSG

SEQ ID NO: 24 - protein, 9 aa, Artificial Sequence
  Synthetic
ADRYGHGSG

SEQ ID NO: 25 - protein, 9 aa, Artificial Sequence
  Synthetic
DRIYYYGSG

SEQ ID NO: 26 - protein, 9 aa, Artificial Sequence
  Synthetic
DKQRIIGSG

SEQ ID NO: 27 - protein, 9 aa, Artificial Sequence
  Synthetic
RYYDYGGSG

SEQ ID NO: 28 - protein, 9 aa, Artificial Sequence
  Synthetic
YRIDRYGSG

SEQ ID NO: 29 - protein, 7 aa, Artificial Sequence
  Synthetic
HYAIGSG

SEQ ID NO: 30 - protein, 7 aa, Artificial Sequence
  Synthetic
FRYYGSG

SEQ ID NO: 31 - protein, 7 aa, Artificial Sequence
  Synthetic
HRRYGSG

SEQ ID NO: 32 - protein, 7 aa, Artificial Sequence
  Synthetic
RYFFGSG

SEQ ID NO: 33 - protein, 7 aa, Artificial Sequence
  Synthetic
DKSIGSG

SEQ ID NO: 34 - protein, 7 aa, Artificial Sequence
  Synthetic
DRNIGSG

SEQ ID NO: 35 - protein, 7 aa, Artificial Sequence
  Synthetic
HYFDGSG

SEQ ID NO: 36 - protein, 7 aa, Artificial Sequence
  Synthetic
YRFDGSG

SEQ ID NO: 37 - protein, 7 aa, Artificial Sequence
  Synthetic
AFNAGSG

SEQ ID NO: 38 - protein, 7 aa, Artificial Sequence
  Synthetic
KFFFGSG

SEQ ID NO: 39 - protein, 7 aa, Artificial Sequence
  Synthetic
AFYHGSG

SEQ ID NO: 40 - protein, 7 aa, Artificial Sequence
  Synthetic
KYGYGSG

SEQ ID NO: 41 - protein, 7 aa, Artificial Sequence
  Synthetic
KYFFGSG

SEQ ID NO: 42 - protein, 7 aa, Artificial Sequence
  Synthetic
HFFAGSG

SEQ ID NO: 43 - protein, 7 aa, Artificial Sequence
  Synthetic
HFIFGSG

SEQ ID NO: 44 - protein, 7 aa, Artificial Sequence
  Synthetic
HNFIGSG

SEQ ID NO: 45 - protein, 7 aa, Artificial Sequence
  Synthetic
YRFFGSG

SEQ ID NO: 46 - protein, 7 aa, Artificial Sequence
  Synthetic
YYFRGSG

SEQ ID NO: 47 - protein, 7 aa, Artificial Sequence
  Synthetic
HYFRGSG

SEQ ID NO: 48 - protein, 9 aa, Artificial Sequence
  Synthetic
GIDQYYGSG

SEQ ID NO: 49 - protein, 9 aa, Artificial Sequence
  Synthetic
HQASSQGSG

SEQ ID NO: 50 - protein, 9 aa, Artificial Sequence
  Synthetic
QQYIIIGSG

SEQ ID NO: 51 - protein, 7 aa, Artificial Sequence
  Synthetic
AIYFGSG

SEQ ID NO: 52 - protein, 7 aa, Artificial Sequence
  Synthetic
NYRSGSG

SEQ ID NO: 53 - protein, 7 aa, Artificial Sequence
  Synthetic
DFNYGSG

SEQ ID NO: 54 - protein, 7 aa, Artificial Sequence
  Synthetic
GSIGGSG

SEQ ID NO: 55 - protein, 7 aa, Artificial Sequence
  Synthetic
GSSYGSG

SEQ ID NO: 56 - protein, 7 aa, Artificial Sequence
  Synthetic
GFYGGSG

SEQ ID NO: 57 - protein, 7 aa, Artificial Sequence
  Synthetic
IAFGGSG

SEQ ID NO: 58 - protein, 7 aa, Artificial Sequence
  Synthetic
IYYAGSG

SEQ ID NO: 59 - protein, 7 aa, Artificial Sequence
  Synthetic
SYIYGSG

SEQ ID NO: 60 - protein, 7 aa, Artificial Sequence
  Synthetic
YAFGGSG